(19) United States
(12) Patent Application Publication
(10) Pub. No.: US 2024/0043384 A1
(43) Pub. Date: Feb. 8, 2024

(54) INHIBITORS OF PU.1 FOR THE TREATMENT OF DISEASE

(71) Applicants: Board of Regents, The University of Texas System, Austin, TX (US); Massachusetts Institute of Technology, Cambridge, MA (US)

(72) Inventors: Barbara CZAKO, Bellaire, TX (US); William RALVENIUS, Somerville, MA (US); Li-Huei TSAI, Cambridge, MA (US)

(21) Appl. No.: 18/481,451

(22) Filed: Oct. 5, 2023

Related U.S. Application Data
(63) Continuation of application No. PCT/US2022/071571, filed on Apr. 6, 2022.
(60) Provisional application No. 63/172,800, filed on Apr. 9, 2021.

(51) Int. Cl. C07D 213/42; C07D 401/12; C07D 217/02; C07D 405/12; C07D 213/61; C07D 213/73; C07D 213/75; C07D 409/12; C07D 413/12; C07D 417/12; C07D 231/56

(52) U.S. Cl. CPC C07D 213/42; C07D 401/12; C07D 217/02; C07D 405/12; C07D 213/61; C07D 213/73; C07D 213/75; C07D 409/12; C07D 413/12; C07D 417/12; C07D 231/56

(57) ABSTRACT

Disclosed herein are compounds which inhibit PU.1, pharmaceutical formulations, and methods of treatment of PU.1-mediated diseases, such as Alzheimer's disease, inflammation, and diseases related to excessive myelin uptake.

This application is a bypass continuation of International Application No. PCT/US2022/071571, filed Apr. 6, 2022, which claims the benefit of priority of U.S. Provisional Application No. 63/172,800 filed Apr. 9, 2021, the disclosures of each are hereby incorporated by reference as if written herein in their entireties.

### GOVERNMENT LICENSE RIGHTS

This invention was made with government support under grant number AG066757 awarded by the National Institutes of Health. The government has certain rights in the invention.

PU.1 is an ETS transcription factor expressed by the SPI1 gene in cells of hematopoietic lineage, including microglia in the brain. The PU.1 protein structure contains a DNA-binding ETS domain, N-terminal acidic & glutamine rich domains that are involved in transcriptional activation, and a PEST domain involved in protein-protein interactions; additionally, there are 3 phosphorylation sites that correlate with activation. In coordination with other cofactors and transcription factors, PU.1 binds to a purine-rich sequence (5′-GAGGAA-3′, the “PU-box”) on enhancers of target genes to activate their transcription. As a transcription factor, PU.1 plays a well-characterized role in regulating myeloid and lymphoid cell fate. In myeloid cells, high levels of PU.1 correspond to maturation of monocytes and differentiation into macrophages. In hematopoietic stem cells (HSCs), PU.1 can activate its own promoter, a process necessary in this cell type to restrict proliferation, but less important for mature cells such as macrophages.

Thus, PU.1 is a key regulator of microglial cells and neuroinflammatory processes during neurodegeneration, however treatments directed to PU.1, or disorders related thereto, are lacking. The present disclosure satisfies this need and provides related advantages as well.

### SUMMARY

Provided herein is a compound of structural Formula (I):

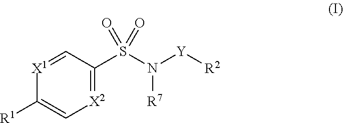

or a pharmaceutically acceptable salt thereof, wherein
Y is a direct bond or CR³R³;
R¹ is chosen from C₁₋₈alkyl, C₁₋₈alkoxy, C₃₋₈cycloalkyl, C₃₋₈cycloalkenyl, C₃₋₈cycloalkoxy, C₆₋₁₀aryl, 5-9 membered heterocycloalkyl, 5-9 membered heterocycloalkyloxy, 5-9 membered heterocycloalkyl(C₁₋₄)alkoxy, and 5-9 membered heteroaryl, each of which is optionally substituted with one or two R⁴ groups;
R² is heteroaryl, optionally substituted with one or two R⁵ groups;
X¹ and X² are chosen from CR₆ and N;
at least one of X¹ and X² is not N;
each occurrence of R³ is independently chosen from H and methyl;
each occurrence of R⁴ is independently chosen from C₁₋₄alkyl, C₁₋₈alkoxy, C₁₋₄alkylcarbonyl, optionally substituted amino, C₃₋₈cycloalkyl, halo, halo C₁₋₄alkyl, halo C₁₋₄alkoxy, hydroxy, and oxo,
   wherein C₁₋₄alkyl and C₁_8alkoxy can be further optionally substituted with one, two or three groups chosen from C₁₋₄alkoxy, optionally substituted amino, hydroxy, and halo;
each occurrence of R⁵ is independently chosen from C₁₋₄ alkyl, C₁₋₈ alkoxy, optionally substituted amino, acylamino, halo, and hydroxy;
each occurrence of R⁶ is independently chosen from hydrogen, halogen, C₁₋₈ alkyl and C₁₋₈ alkoxy; and
R⁷ is chosen from hydrogen and C₁₋₄ alkyl;
provided that when R¹ is cyclopentyl or cyclohexyl, each of which is optionally substituted with one or two R⁴ groups, and R² is pyridin-4-yl optionally substituted with one or two R⁵ groups, then Y is not CH₂; and
further provided that when R¹ is methoxy or ethoxy and R² is pyridin-4-yl, then Y is not CH₂.

Also provided herein is a compound of structural Formula (I):

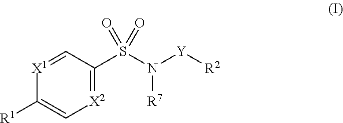

or a pharmaceutically acceptable salt thereof, wherein
Y is a direct bond or CR³R³;
R¹ is chosen from C₁₋₈alkyl, C₃_8alkoxy, C₃₋₈cycloalkenyl, C₃₋₈cycloalkoxy, C₆₋₁₀aryl, 5-9 membered heterocycloalkyl, 5-9 membered heterocycloalkyloxy, 5-9 membered heterocycloalkyl(C₁₋₄)alkoxy, and 5-9 membered heteroaryl, each of which is optionally substituted with one or two R⁴ groups or R¹ is C₃₋₈cycloalkyl substituted with one or two R⁴ groups;
R² is heteroaryl, optionally substituted with one or two R⁵ groups;
X¹ and X² are chosen from CR⁶, and N, provided that at least one of X¹ and X² is not N;
each occurrence of R³ is independently chosen from H and methyl;
each occurrence of R⁴ is independently chosen from C₁₋₄alkyl, C₁₋₈alkoxy, C₁₋₄alkylcarbonyl, optionally substituted amino, C₃₋₈cycloalkyl, halo, halo C₁₋₄alkyl, halo C₁₋₄alkoxy, hydroxy, and oxo,
   wherein C₁₋₄alkyl and C₁₋₈alkoxy can be further optionally substituted with one, two or three groups chosen from C₁₋₄alkoxy, optionally substituted amino, hydroxy, and halo;
each occurrence of R⁵ is independently chosen from C₁₋₄ alkyl, C₁₋₈ alkoxy, optionally substituted amino, halo, and hydroxy;
each occurrence of R⁶ is independently chosen from hydrogen, C₁₋₈ alkyl and C₁₋₈alkoxy; and
R⁷ is chosen from hydrogen and C₁₋₄ alkyl.

Also provided is a pharmaceutical formulation comprising a compound described herein, or a pharmaceutically acceptable salt thereof, and a pharmaceutically acceptable carrier.

Also provided is a method of treatment of a PU.1-mediated disease comprising the administration of a therapeutically effective amount of a compound described herein, or a pharmaceutically acceptable salt thereof, to a patient in need thereof.

These and other aspects of the disclosure disclosed herein will be set forth in greater detail as the patent disclosure proceeds.

### DETAILED DESCRIPTION

As used in the present specification, the following words and phrases are generally intended to have the meanings as set forth below, except to the extent that the context in which they are used indicates otherwise.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant FIGURES (e.g., 1.255 μM, 2.1 μM, 2.9999 μM, etc.).

The term “about,” as used herein, is intended to qualify the numerical values which it modifies, denoting such a value as variable within a margin of error. When no particular margin of error, such as a standard deviation to a mean value given in a chart or table of data, is recited, the term “about” should be understood to mean that range which would encompass the recited value and the range which would be included by rounding up or down to that FIGURE as well, taking into account significant FIGURES.

The term “acyl,” as used herein, alone or in combination, refers to a carbonyl attached to an alkenyl, alkyl, aryl, cycloalkyl, heteroaryl, heterocycle, or any other moiety were the atom attached to the carbonyl is carbon. An “acetyl” group refers to a —C(O)CH₃ group. An “alkylcarbonyl” or “alkanoyl” group refers to an alkyl group attached to the parent molecular moiety through a carbonyl group. Examples of such groups include methylcarbonyl and ethylcarbonyl.

The term “alkenyl,” as used herein, alone or in combination, refers to a straight-chain or branched-chain hydrocarbon radical having one or more double bonds and containing from 2 to 20 carbon atoms. In some embodiments, alkenyl will comprise from 2 to 6 carbon atoms. The term “alkenylene” refers to a carbon-carbon double bond system attached at two or more positions such as ethenylene [(—CH═CH—), (—C::C—)]. Examples of suitable alkenyl radicals include ethenyl, propenyl, 2-methylpropenyl, 1,4-butadienyl and the like. Unless otherwise specified, the term “alkenyl” may include “alkenylene” groups.

The term “alkoxy”, and, interchangeably, “(alkyl)oxy”, as used herein, alone or in combination, refers to an alkyl radical attached to a molecule by oxygen. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain saturated, hydrocarbon radical containing from 1 to 20 carbon atoms. In some embodiments, alkyl will comprise from 1 to 10 carbon atoms. In some embodiments, alkyl will comprise from 1 to 8 carbon atoms. Examples of alkyl radicals include methyl, ethyl, n-propyl, isopropyl, n-butyl, isobutyl, sec-butyl, tert-butyl, pentyl, iso-amyl, hexyl, octyl, noyl and the like. The term “alkylene,” as used herein, alone or in combination, refers to a straight chain saturated hydrocarbon attached at two or more positions, such as methylene (—CH₂—), ethylene (—CH₂CH₂—), and propylene (—CH₂ CH₂CH₂—). “Alkylene” can thus be described as —(CH₂)ₙ— with n being an positive integer. In some embodiments, n is chosen from 1 to 20. In some embodiments, n is chosen from 1 to 10. In some embodiments, n is chosen from 1 to 8. In some embodiments, n is chosen from 1 to 6. Unless otherwise specified, the term “alkyl” may include “alkylene” groups.

An “alkylcarbonyl” or “alkanoyl” group refers to an alkyl group attached to the parent molecular moiety through a carbonyl group. Examples of such groups include methylcarbonyl and ethylcarbonyl.

The term “amino,” as used herein, alone or in combination, refers to —NH₂.

The term “substituted amino,” as used herein, alone or in combination, refers to —NRR′ wherein R and R′ are independently chosen from hydrogen, alkyl, acyl, heteroalkyl, aryl, cycloalkyl, heteroaryl, and heterocycloalkyl, provided that both R and R′ cannot be hydrogen.

The term “aryl,” as used herein, alone or in combination, means a carbocyclic aromatic system containing one, two or three rings wherein such polycyclic ring systems are fused together. The term “aryl” embraces aromatic groups such as phenyl, naphthyl, anthracenyl, and phenanthryl.

The term “arylalkyl” or “aralkyl,” as used herein, alone or in combination, refers to an aryl group attached to the parent molecular moiety through an alkyl group.

The term “cycloalkyl,” or, alternatively, “carbocycle,” as used herein, alone or in combination, refers to a saturated monocyclic, bicyclic or tricyclic alkyl group wherein each cyclic moiety contains from 3 to 12 carbon atom ring members. In some embodiments, cycloalkyl will comprise from 5 to 7 carbon atoms. In some embodiments, cycloalkyl will comprise a spirocycle ring system. Examples of such cycloalkyl groups include cyclopropyl, cyclobutyl, cyclopentyl, cyclohexyl, cycloheptyl, and the like. “Bicyclic” and “tricyclic” as used herein are intended to include both fused ring systems, as well as the multicyclic (multicentered) saturated type.

The term “cycloalkenyl,” as used herein alone or in combination, refers to an unsaturated, non-aromatic, cycloalkyl group.

The term “cycloalkoxy,” as used herein alone or in combination, refers to a cycloalkyl group attached to the parent molecular moiety through an oxygen atom.

The term “halo,” or “halogen,” as used herein, alone or in combination, refers to fluorine, chlorine, bromine, or iodine.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical as defined herein wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical. Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl.

The term “heteroaryl,” as used herein, alone or in combination, refers to a 3 to 15 membered unsaturated heteromonocyclic ring, or a fused monocyclic, bicyclic, or tricyclic ring system in which at least one of the fused rings is aromatic, which contains at least one atom chosen from N, O, and S. In some embodiments, heteroaryl will comprise from 1 to 4 heteroatoms as ring members. In some embodiments, heteroaryl will comprise from 1 to 2 heteroatoms as ring members. In some embodiments, heteroaryl will comprise from 5 to 7 atoms. The term also embraces fused polycyclic groups wherein heterocyclic rings are fused with aryl rings wherein heteroaryl rings are fused with other heteroaryl rings wherein heteroaryl rings are fused with heterocycloalkyl rings, or wherein heteroaryl rings are fused with cycloalkyl rings. Examples of heteroaryl groups include pyrrolyl, pyrrolinyl, imidazolyl, pyrazolyl, pyridinyl, pyrimidinyl, pyrazinyl, pyridazinyl, triazolyl, pyranyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuryl, benzothienyl, chromonyl, coumarinyl, benzopyranyl, tetrazolopyridazinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like.

The terms “heterocycloalkyl” and, interchangeably, “heterocycle,” as used herein, alone or in combination, each refer to a saturated, partially unsaturated, or fully unsaturated (but nonaromatic) monocyclic; saturated, partially unsaturated, or fully unsaturated (but not fully aromatic) bicyclic; or saturated, partially unsaturated, or fully unsaturated (but not fully aromatic) tricyclic heterocyclic group containing at least one heteroatom as a ring member wherein each heteroatom may be independently chosen from nitrogen, oxygen, and sulfur. The term includes polycyclic groups which comprise at least one nonaromatic ring, such as 1,2-dihydroquinoline, 5,6-dihydroquinoline, and 2,3-dihydrobenzofuran. The term excludes polycyclic groups in which every ring is nonaromatic, such as indole, quinoline, and acridine.

In some embodiments, heterocycloalkyl will comprise a spirocycle ring system. In some embodiments, hetercycloalkyl will comprise from 1 to 4 heteroatoms as ring members. In some embodiments, hetercycloalkyl will comprise from 1 to 2 heteroatoms as ring members. In some embodiments, hetercycloalkyl will comprise from 3 to 8 ring members in each ring. In some embodiments, hetercycloalkyl will comprise from 3 to 7 ring members in each ring. In some embodiments, hetercycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl or heteroaryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like.

The term “heterocycloalkoxy” or “heterocycloalkyloxy,” as used herein alone or in combination, refers to a heterocycloalkyl group attached to the parent molecular moiety through an oxygen atom.

The term “lower,” as used herein, alone or in a combination, where not otherwise specifically defined, means containing from 1 to and including 6 carbon atoms (i.e., C₁-C₆ alkyl).

The term “oxo,” as used herein, alone or in combination, refers to ═O.

The term “spirocycle ring system” refers to a polycyclic ring system comprising two rings such that a single atom is common to both rings.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

Asymmetric centers exist in the compounds and pharmaceutically acceptable salts thereof, disclosed herein. These centers are designated by the symbols “R” or “S,” depending on the configuration of substituents around the chiral carbon atom. It should be understood that the disclosure encompasses all stereochemical isomeric forms, including diastereomeric, enantiomeric, and epimeric forms, as well as d-isomers and 1-isomers, and mixtures thereof. Individual stereoisomers of compounds, and pharmaceutically acceptable salts thereof, can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds, and pharmaceutically acceptable salts thereof, of particular stereochemistry are either commercially available or can be made and resolved by techniques known in the art. Additionally, the compounds, and pharmaceutically acceptable salts thereof, disclosed herein may exist as geometric isomers. The present disclosure includes all cis, trans, syn, anti, entgegen (E), and zusammen (Z) isomers as well as the appropriate mixtures thereof.

Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

The term “disease” as used herein is intended to be generally synonymous, and is used interchangeably with, the terms “disorder,” “syndrome,” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

The term “combination therapy” means the administration of two or more therapeutic agents to treat a therapeutic condition or disorder described in the present disclosure. Such administration encompasses co-administration of these therapeutic agents in a substantially simultaneous manner, such as in a single capsule having a fixed ratio of active ingredients or in multiple, separate capsules for each active ingredient. In addition, such administration also encompasses use of each type of therapeutic agent in a sequential manner. In either case, the treatment regimen will provide beneficial effects of the drug combination in treating the conditions or disorders described herein.

“PU.1 binder” is used herein to refer to a compound, or a pharmaceutically acceptable salt thereof, that exhibits an K_{d} with respect to PU.1 of no more than about 100 μM and more typically not more than about 50 μM, as measured in the PU.1 binding assay described generally herein. The PU.1 binding assay measures the K_{d} (dissociation constant) for the binding of a compound, or a pharmaceutically acceptable salt thereof, with the active site of PU.1. Certain compounds disclosed herein, or a pharmaceutically acceptable salt thereof, have been discovered to bind to PU.1. In some embodiments, compounds, or pharmaceutically acceptable salts thereof, will exhibit an K_{d} with respect to PU.1 of no more than about 10 μM; in some embodiments, compounds, or pharmaceutically acceptable salts thereof, will exhibit a K_{d} with respect to PU.1 of no more than about 1 μM; in some embodiments, compounds, or pharmaceutically acceptable salts thereof, will exhibit a K_{d} with respect to PU.1 of not more than about 0.1 μM; in some embodiments, compounds, or salts or tautome thereof, will exhibit a K_{d} with respect to PU.1 of not more than about 10 nM, as measured in the PU.1 assay described herein.

The phrase “therapeutically effective” is intended to qualify the amount of active ingredients used in the treatment of a disease or disorder or on the effecting of a clinical endpoint.

The term “therapeutically acceptable” refers to those compounds (or salts thereof) which are suitable for use in contact with the tissues of patients without undue toxicity, irritation, and allergic response, are commensurate with a reasonable benefit/risk ratio, and are effective for their intended use.

As used herein, reference to “treatment” of a patient is intended to include prophylaxis. Treatment may also be preemptive in nature, i.e., it may include prevention of disease. Prevention of a disease may involve complete protection from disease, for example as in the case of prevention of infection with a pathogen, or may involve prevention of disease progression. For example, prevention of a disease may not mean complete foreclosure of any effect related to the diseases at any level, but instead may mean prevention of the symptoms of a disease to a clinically significant or detectable level. Prevention of diseases may also mean prevention of progression of a disease to a later stage of the disease.

The term “patient” is generally synonymous with the term “subject” and includes all mammals including humans Examples of patients include humans, livestock such as cows, goats, sheep, pigs, and rabbits, and companion animals such as dogs, cats, rabbits, and horses. Preferably, the patient is a human.

Those skilled in the art will appreciate that the invention(s) described herein is susceptible to variations and modifications other than those specifically described. It is to be understood that the invention(s) includes all such variations and modifications. The invention(s) also includes all the steps, features, compositions and compounds referred to or indicated in this specification, individually or collectively, and any and all combinations or any two or more of steps or features unless specifically stated otherwise.

The present invention(s) is not to be limited in scope by the specific embodiments described herein, which are intended for the purpose of exemplification only. Functionally equivalent products, compositions, and methods are clearly within the scope of the invention(s), as described herein.

It is appreciated that certain features of the invention(s), which are, for clarity, described in the context of separate embodiments, can also be provided in combination in a single embodiment. Conversely, various features of the invention(s), which are, for brevity, described in the context of a single embodiment, can also be provided separately or in any suitable subcombination.

### List of Abbreviations

Ac₂O=acetic anhydride; AcCl=acetyl chloride; AcOH=acetic acid; AIBN=azobisisobutyronitrile; aq.=aqueous; B₂pin₂=bis(pinacolato)diboron=4,4,5,5-tetramethyl-2-(4,4,5,5-tetramethyl-1,3,2-dioxaborolan-2-yl)-1,3,2-dioxaborolane; BPO=benzoyl peroxide; Bu=butyl; CD₃OD=deuterated methanol; CDCl₃=deuterated chloroform; CDI=1,1′-carbonyldiimidazole; dba=dibenzylideneacetone; DCE=1,2-dichloroethane; DCM=dichloromethane; DIEA=DIPEA=N,N-diisopropylethylamine; DMAP=4-dimethylaminopyridine; DMF=N,N-dimethylformamide; DMSO-d₆=deuterated dimethyl sulfoxide; DMSO=dimethyl sulfoxide; dppf=1,1′-bis(diphenylphosphino)ferrocene; Et=ethyl; Et₂O=diethyl ether; EtOAc=ethyl acetate; EtOH=ethanol; h=hr=hour; HOBT=1-hydroxybenzotriazole; iPr=i-Pr=isopropyl=2-propyl; iPrOH=i-PrOH=isopropanol; LAH=lithium aluminiumhydride; LDA=lithium diisopropyl amide; MeCN=acetonitrile; MeI=methyl iodide; MeOH=methanol; MP-carbonate resin=macroporous triethylammonium methylpolystyrene carbonate resin; MsCl=mesyl chloride; MTBE=methyl tert-butyl ether; n-BuLi=n-butyllithium; NaOEt=sodium ethoxide; NaOMe=sodium methoxide; NaOtBu=sodium t-butoxide; NBS=N-bromosuccinimide; NCS=N-chlorosuccinimide; NIS=N-iodosuccinimide; Pd(PPh₃)₄=tetrakis(triphenylphosphine)-palladium(0); Pd₂dba₃=Pd₂(dba)₃=tris(dibenzylideneacetone)dipalladium(0); PdCl₂(PPh₃)₂=bis(triphenylphosphine)palladium(II) dichloride; PG=protecting group; Ph=phenyl; prep-HPLC=preparative high-performance liquid chromatography; PMB=para-methoxybenzyl; PMBCl=para-methoxybenzyl chloride; PMBOH=para-methoxybenzyl alcohol; Pyr=pyridine; RT=room temperature; RuPhos=2-dicyclohexylphosphino-2′,6′-diisopropoxybiphenyl; sat.=saturated; tBu=t-Bu=tert-butyl=1,1-dimethylethyl; t-BuOH=tBuOH=tert-butanol; TEA=Et₃N=triethylamine; TFA=trifluoroacetic acid; TFAA=trifluoroacetic anhydride; THE=tetrahydrofuran; Tol=toluene; TsCl=tosyl chloride; Xantphos=4,5-Bis(diphenylphosphino)-9,9-dimethylxanthene; XPhos=2-dicyclohexylphosphino-2′,4′, 6′-triisopropylbiphenyl.
CD14=cluster of differentiation 14, CO₂=carbon dioxide, DMSO=dimethyl sulfoxide, ELISA=enzyme-linked immunosorbent assay, hr=hour, M=molar, min=minute, mL=milliliter, ng=nanograms, PBS=phosphate buffered saline, PMA=phorbol 12-myristate 13-acetate, rcf=relative centrifugal force, rpm=rotations per minute, rt=room temperature, TMB=3,3′,5,5′-Tetramethylbenzidine, μl=microliter

Provided is a compound of structural Formula (I):

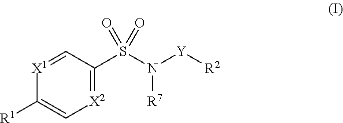

or a pharmaceutically acceptable salt thereof, wherein
Y is a direct bond or CR³R³;
R¹ is chosen from C₁₋₈alkyl, C₁₋₈alkoxy, C₃₋₈cycloalkyl, C₃₋₈cycloalkenyl, C₃₋₈cycloalkoxy, C₆₋₁₀aryl, 5-9 membered heterocycloalkyl, 5-9 membered heterocycloalkyloxy, 5-9 membered heterocycloalkyl(C₁₋₄)alkoxy, and 5-9 membered heteroaryl, each of which is optionally substituted with one or two R⁴ groups;
R² is heteroaryl, optionally substituted with one or two R⁵ groups; X¹ and X² are chosen from CR₆ and N;
at least one of X¹ and X² is not N;
each occurrence of R³ is independently chosen from H and methyl;
each occurrence of R⁴ is independently chosen from C₁₋₄alkyl, C₁₋₈alkoxy, C₁₋₄alkylcarbonyl, optionally substituted amino, C₃₋₈cycloalkyl, halo, halo C₁₋₄alkyl, halo C₁₋₄alkoxy, hydroxy, and oxo,
   wherein C₁₋₄alkyl and C₁₋₈alkoxy can be further optionally substituted with one, two or three groups chosen from C₁₋₄alkoxy, optionally substituted amino, hydroxy, and halo;
each occurrence of R⁵ is independently chosen from C₁₋₄ alkyl, C₁₋₈ alkoxy, optionally substituted amino, acylamino, halo, and hydroxy;
each occurrence of R⁶ is independently chosen from hydrogen, halogen, C₁₋₈ alkyl and C₁₋₈ alkoxy; and
R⁷ is chosen from hydrogen and C₁₋₄ alkyl;
provided that when R¹ is cyclopentyl or cyclohexyl, each of which is optionally substituted with one or two R⁴ groups, and R² is pyridin-4-yl optionally substituted with one or two R⁵ groups, then Y is not CH₂; and
further provided that when R¹ is methoxy or ethoxy and R² is pyridin-4-yl, then Y is not CH₂.

Also provided is a compound of Formula I

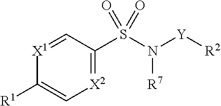

or a pharmaceutically acceptable salt thereof, wherein
Y is a direct bond or CR³R³;
R¹ is chosen from C₁₋₈alkyl, C₃₋₈alkoxy, C₃₋₈cycloalkenyl, C₃₋₈cycloalkoxy, C₆₋₁₀aryl, 5-9 membered heterocycloalkyl, 5-9 membered heterocycloalkyloxy, 5-9 membered heterocycloalkyl(C₁₋₄)alkoxy, and 5-9 membered heteroaryl, each of which is optionally substituted with one or two R⁴ groups or R¹ is C₃₋₈cycloalkyl substituted with one or two R⁴ groups;
R² is heteroaryl, optionally substituted with one or two R⁵ groups;
X¹ and X² are chosen from CR⁶, and N, provided that at least one of X¹ and X² is not N;
each occurrence of R³ is independently chosen from H and methyl;
each occurrence of R⁴ is independently chosen from C₁₋₄alkyl, C₁₋₈alkoxy, C₁₋₄alkylcarbonyl, optionally substituted amino, C₃₋₈cycloalkyl, halo, halo C₁₋₄alkyl, halo C₁₋₄alkoxy, hydroxy, and oxo,
   wherein C₁₋₄alkyl and C₁₋₈alkoxy can be further optionally substituted with one, two or three groups chosen from C₁₋₄alkoxy, optionally substituted amino, hydroxy, and halo;
each occurrence of R⁵ is independently chosen from C₁₋₄ alkyl, C₁₋₈ alkoxy, optionally substituted amino, halo, and hydroxy;
each occurrence of R⁶ is independently chosen from hydrogen, C₁₋₈ alkyl and C₁₋₈alkoxy; and
R⁷ is chosen from hydrogen and C₁₋₄ alkyl.

In some embodiments, R¹ is C₁₋₈alkyl, optionally substituted with one or two R⁴ groups. In some embodiments, R¹ is C₁₋₄alkyl. In some embodiments, R¹ is 2-propyl.

In some embodiments, R¹ is C₁₋₈alkoxy, optionally substituted with one or two R⁴ groups. In some embodiments, R¹ is chosen from ethoxy, 2-(trifluoromethoxy)ethoxy, n-propoxy, 3-hydroxypropoxy, 3-methoxypropoxy, 3-(trifluoromethoxy)propoxy, 2-methoxyethoxy, 2-n-butoxy, n-pentoxy, and 3-hydroxy-3-methylbutoxy.

In some embodiments, R¹ is C₃₋₈alkoxy, optionally substituted with one or two R⁴ groups. In some embodiments, R¹ is chosen from n-propoxy, 3-hydroxypropoxy, 3-methoxypropoxy, 3-(trifluoromethoxy)propoxy, 2-methoxyethoxy, 2-n-butoxy, n-pentoxy, and 3-hydroxy-3-methylbutoxy.

In some embodiments, R¹ is cycloalkyl, optionally substituted with one or two R⁴ groups. In some embodiments, R¹ is chosen from cyclobutyl, cyclopentyl, and cyclohexyl, optionally substituted with one or two R⁴ groups.

In some embodiments, R¹ is C₃₋₈cycloalkyl substituted with one or two R⁴ groups. In some embodiments, R¹ is chosen from cyclobutyl, cyclopentyl, cyclohexyl,

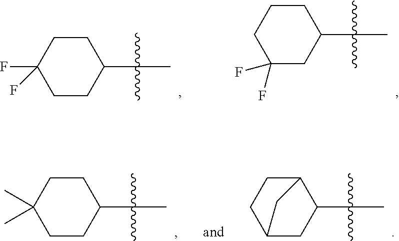

In some embodiments, R¹ is C₃₋₈cycloalkenyl, optionally substituted with one or two R⁴ groups. In some embodiments, R¹ is chosen from

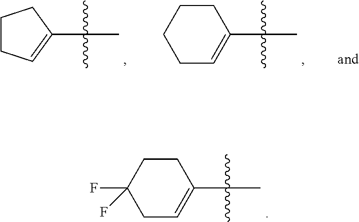 [SMILES: *C1=CCCC1 ; *C1=CCCCC1 ; *C1=CCC(F)(F)CC1]

In some embodiments, R¹ is C₁₋₈cycloalkoxy, optionally substituted with one or two R⁴ groups. In some embodiments, R¹ is chosen from

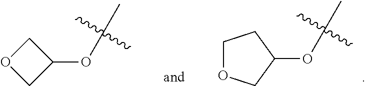 [SMILES: *OC1COC1 ; *OC1CCOC1]

In some embodiments, R¹ is aryl, optionally substituted with one or two R⁴ groups. In some embodiments, R¹ is phenyl, optionally substituted with one or two R⁴ groups. In some embodiments, R¹ is phenyl, 3-ethyl-4-ethoxyphenyl, 3-methyl-4-propoxyphenyl, 3-methyl-4-(2-methoxyethoxy)phenyl, 3-methyl-4-(2-(dimethylamino)ethoxy)phenyl, 3-cyclopropyl-4-ethoxyphenyl, 3,3-dimethyl-2,3-dihydro-1H-inden-5-yl, 3-oxo-2,3-dihydro-1H-inden-5-yl, and bicyclo[4.2.0]octa-1,3,5-trien-3-yl.

In some embodiments, R¹ is 5-9 membered heterocycloalkyl group chosen from:

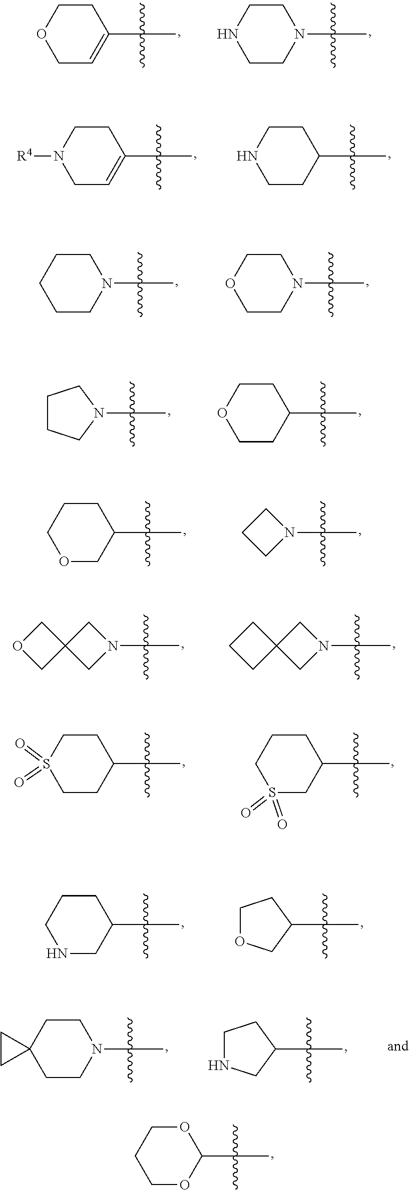

each of which is optionally substituted with one or two R⁴ groups.

In some embodiments, R¹ is 5-9 membered heterocycloalkyl group chosen from:

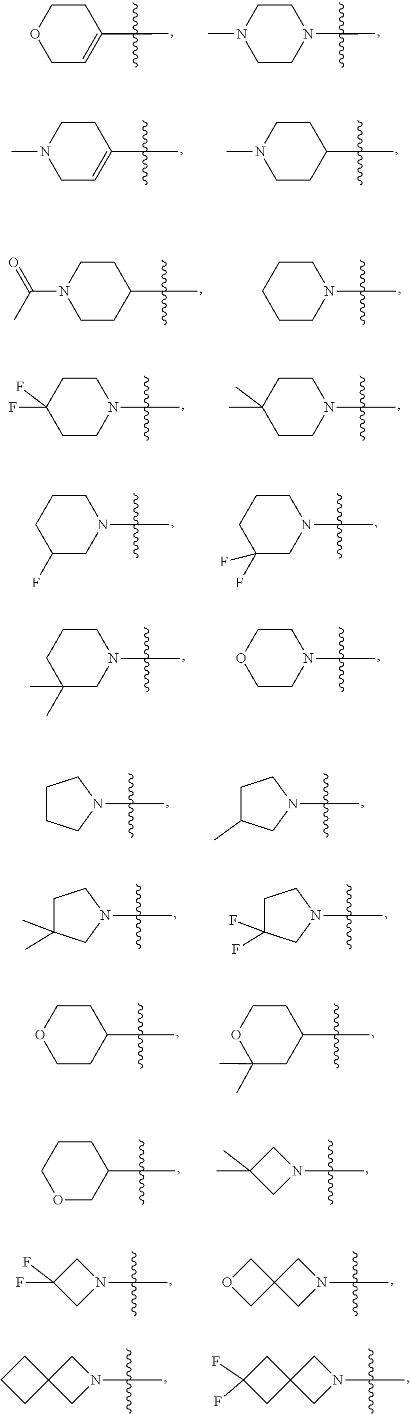

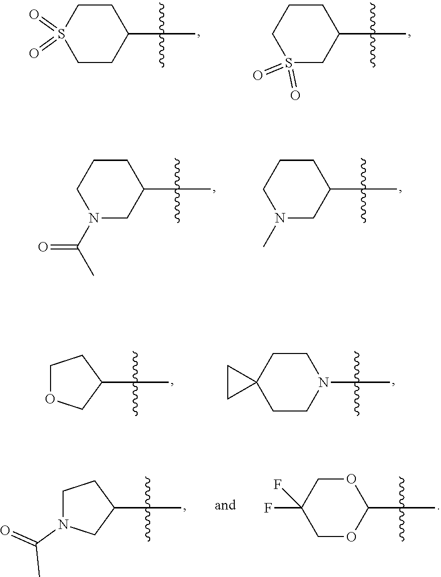

In some embodiments, R¹ is −9 membered heterocycloalkyl(C₁₋₄)alkoxy. In some embodiments, R¹ is chosen from

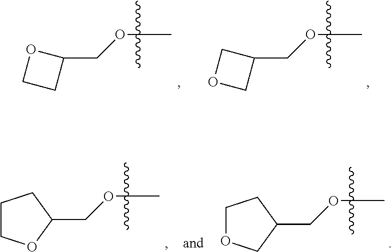 [SMILES: *OCC1CCO1 ; *OCC1COC1 ; *OCC1CCCO1 ; *OCC1CCOC1]

In some embodiments, R¹ is heteroaryl, optionally substituted with one or two R⁴ groups. In some embodiments, R¹ is chosen from:

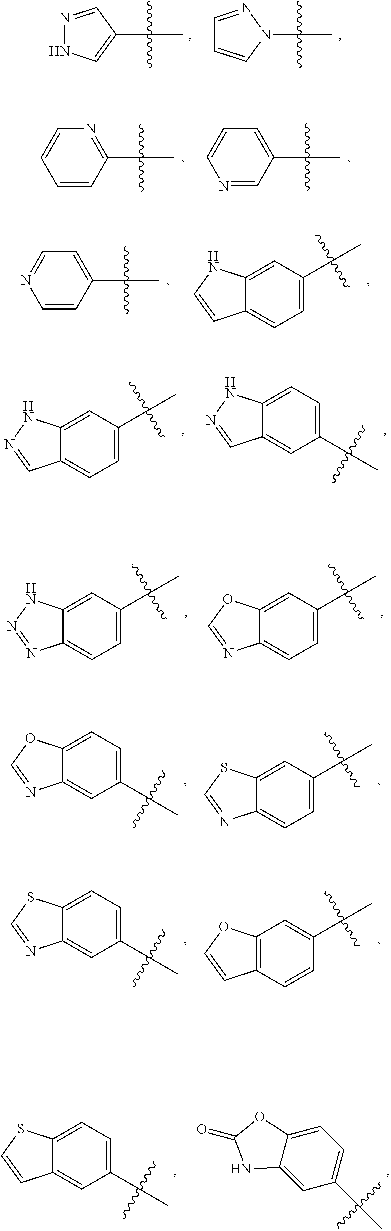

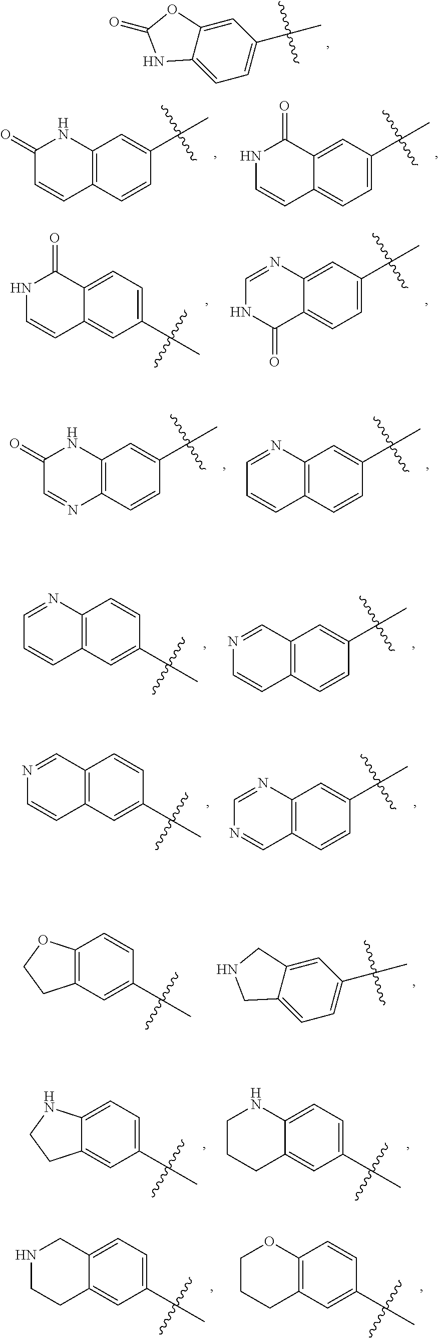

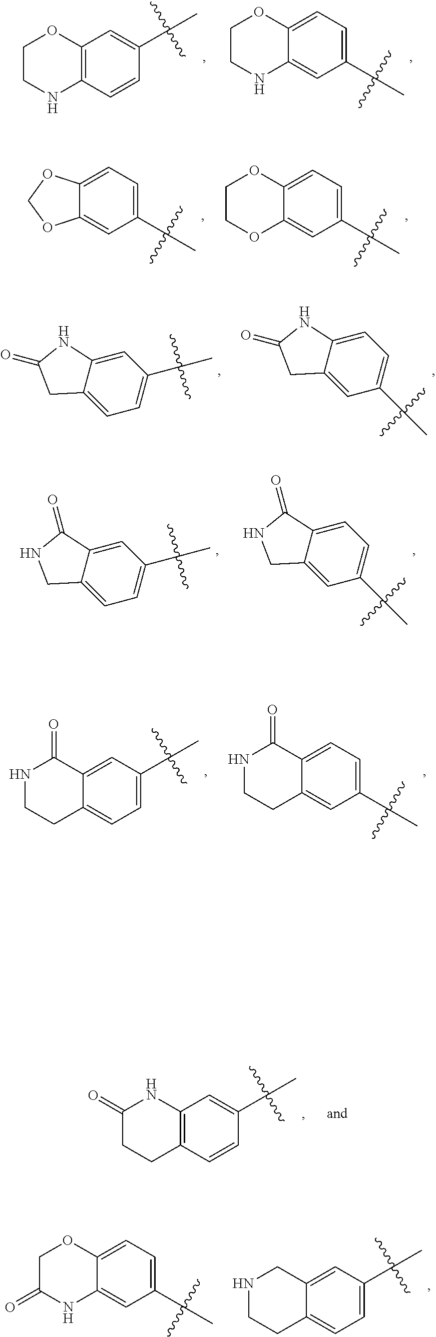

each of which is optionally substituted with one or two R⁴ groups. In some embodiments, R¹ is chosen from:

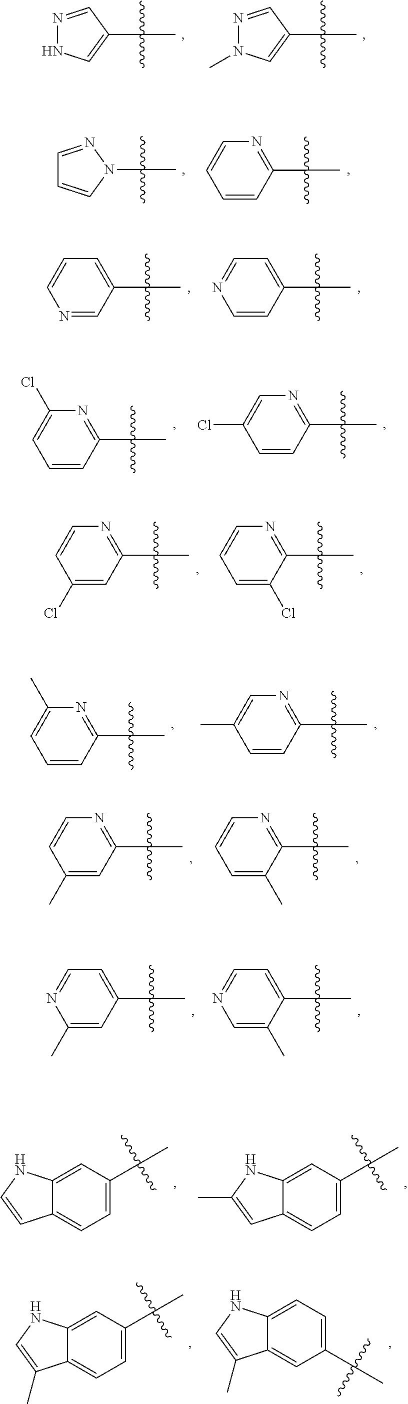

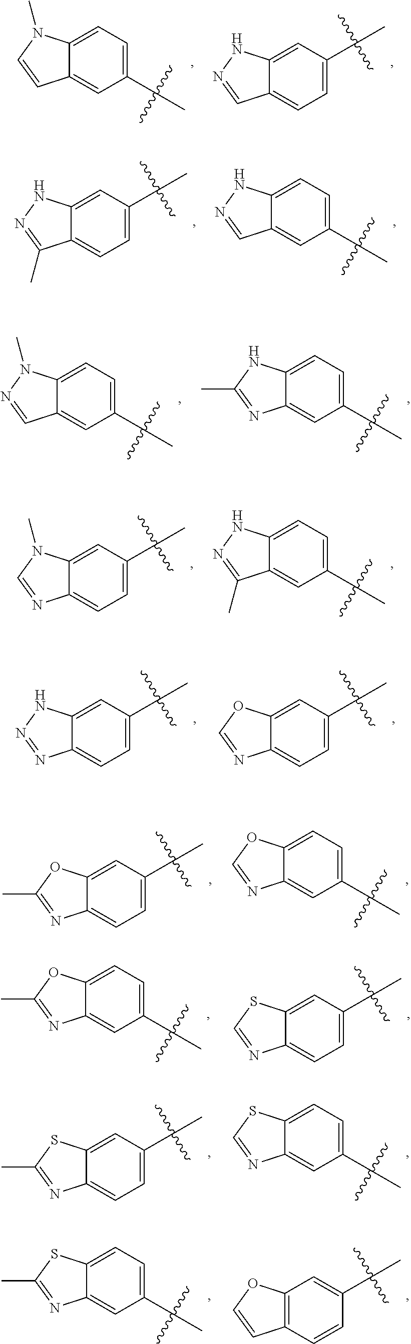

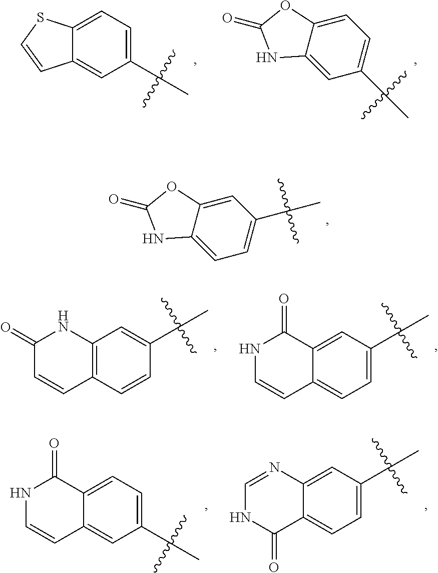

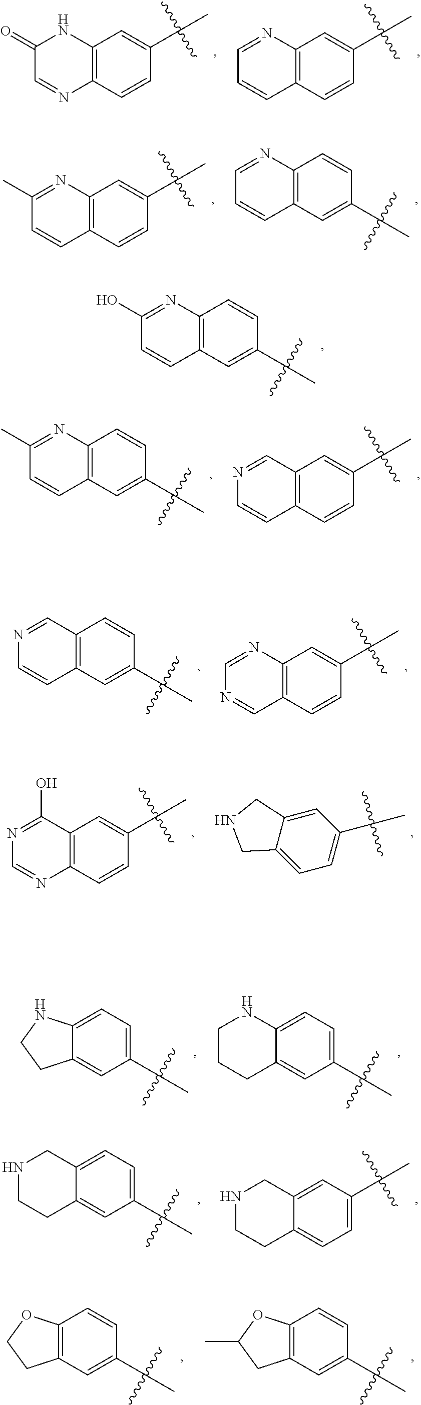

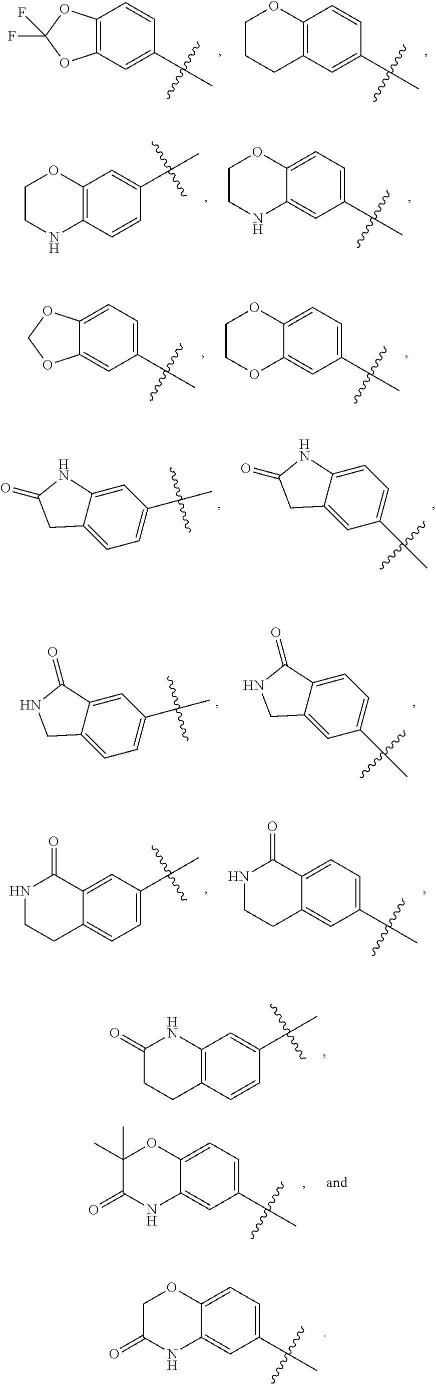

In some embodiments, R² is chosen from pyridin-4-yl, isoquinolin-5-yl, 1H-indazol-7-yl, 1H-indazol-4-yl, 1H-pyrrolo[2,3-b]pyridin-4-yl, 1-methyl-1H-pyrazol-5-yl, pyridin-4-yl, 2-fluoropyridin-4-yl, 3-fluoropyridin-4-yl, 2-aminopyridin-4-yl, 3-aminopyridin-4-yl, 2-methoxypyridin-4-yl, 3-methoxypyridin-4-yl, 2-pentoxypyridin-4-yl, 3-pentoxypyridin-4-yl, 2-chloropyridin-4-yl, 3-chloropyridin-4-yl, 2-methylpyridin-4-yl, 3-methylpyridin-4-yl, isoquinolin-5-yl, 3-chloro-1-methyl-1H-indazol-7-yl, 3-chloro-1H-indazol-7-yl, 1-methyl-1H-indazol-4-yl, 1-methyl-1H-indol-4-yl, 3-methyl-1H-indol-7-yl, 1-methyl-1H-pyrrolo[2,3-b]pyridin-4-yl, and 1H-pyrrolo[2,3-b]pyridin-4-yl, each of which is optionally substituted with one or two R⁵ groups.

In some embodiments, R² is chosen from 1-methyl-1H-pyrazol-5-yl, pyridin-4-yl, 2-fluoropyridin-4-yl, 3-fluoropyridin-4-yl, 2-aminopyridin-4-yl, 3-aminopyridin-4-yl, 2-methoxypyridin-4-yl, 3-methoxypyridin-4-yl, 2-pentoxypyridin-4-yl, 3-pentoxypyridin-4-yl, 2-chloropyridin-4-yl, 3-chloropyridin-4-yl, 2-methylpyridin-4-yl, 3-methylpyridin-4-yl, isoquinolin-5-yl, 3-chloro-1-methyl-1H-indazol-7-yl, 3-chloro-1H-indazol-7-yl, 1-methyl-1H-indazol-4-yl, 1-methyl-1H-indol-4-yl, 3-methyl-1H-indol-7-yl, 1-methyl-1H-pyrrolo[2,3-b]pyridin-4-yl, and 1H-pyrrolo[2,3-b]pyridin-4-yl.

In some embodiments, R² is pyridin-4-yl, optionally substituted with one R⁵ groups. In some embodiments, R² is pyridin-4-yl.

In some embodiments, Y is a direct bond.

In some embodiments, Y is CR³R³.

In some embodiments, at least one occurrence of R³ is H. In some embodiments, both occurrences of R³ are H. In some embodiments, one occurrence of R³ is H and the other is methyl. In some embodiments, both occurrences of R³ are methyl.

In some embodiments, each occurrence R⁴ is independently chosen from methyl, methoxy, hydroxy, oxo, chloro, and fluoro. In some embodiments, each occurrence R⁵ is independently chosen from chloro, fluoro, methyl, and methoxy.

Also provided is a compound chosen from:

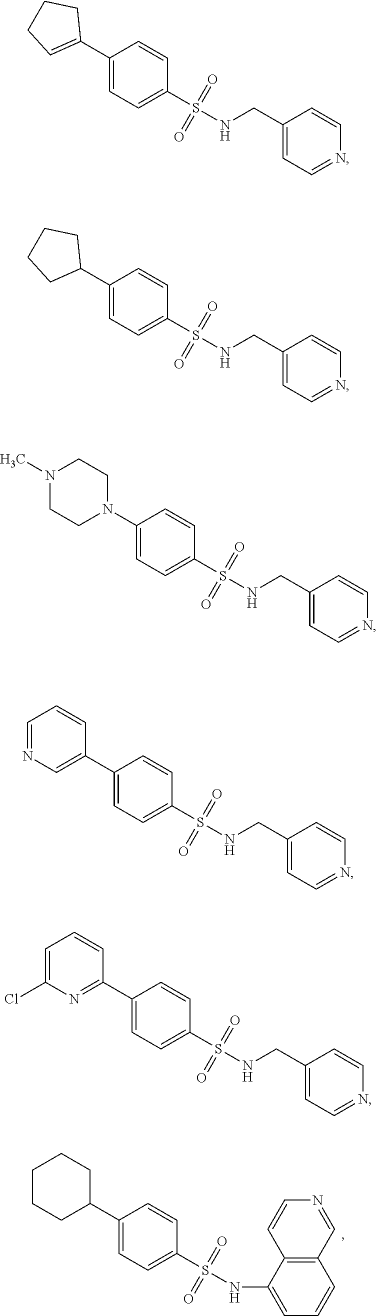 [SMILES: O=S(=O)(NCc1ccncc1)c1ccc(C2=CCCC2)cc1 ; O=S(=O)(NCc1ccncc1)c1ccc(C2CCCC2)cc1 ; CN1CCN(c2ccc(S(=O)(=O)NCc3ccncc3)cc2)CC1 ; O=S(=O)(NCc1ccncc1)c1ccc(-c2cccnc2)cc1 ; O=S(=O)(NCc1ccncc1)c1ccc(-c2cccc(Cl)n2)cc1 ; O=S(=O)(Nc1cccc2cnccc12)c1ccc(C2CCCCC2)cc1]

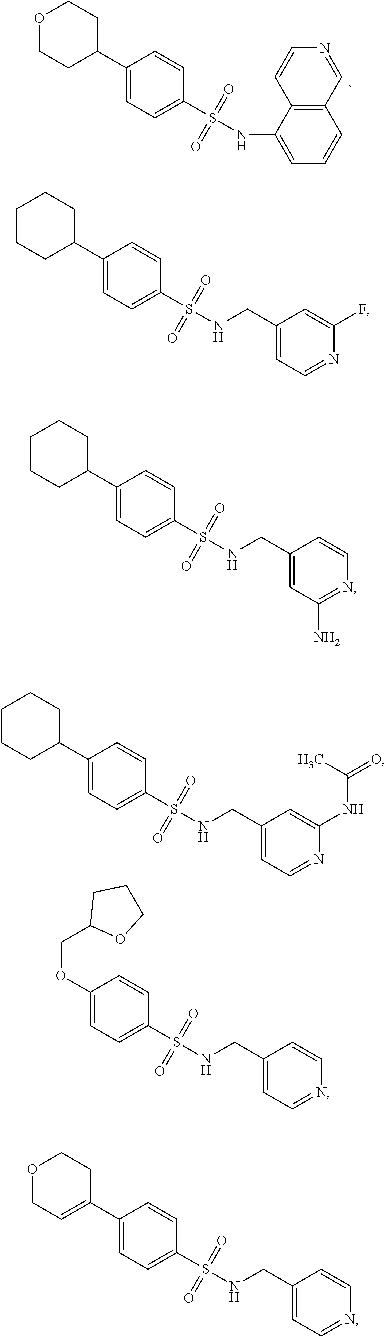 [SMILES: O=S(=O)(Nc1cccc2cnccc12)c1ccc(C2CCOCC2)cc1 ; O=S(=O)(NCc1ccnc(F)c1)c1ccc(C2CCCCC2)cc1 ; Nc1cc(CNS(=O)(=O)c2ccc(C3CCCCC3)cc2)ccn1 ; CC(=O)Nc1cc(CNS(=O)(=O)c2ccc(C3CCCCC3)cc2)ccn1 ; O=S(=O)(NCc1ccncc1)c1ccc(OCC2CCCO2)cc1 ; O=S(=O)(NCc1ccncc1)c1ccc(C2=CCOCC2)cc1]

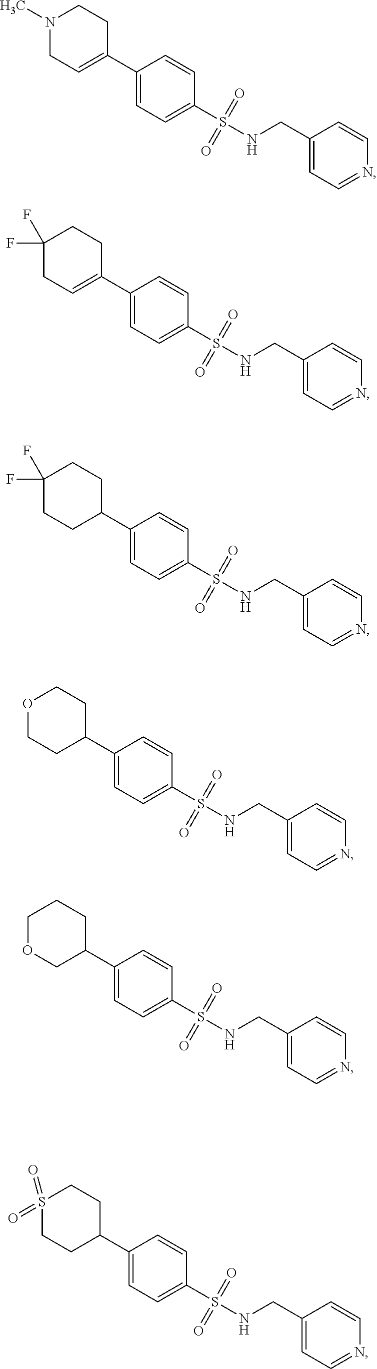 [SMILES: CN1CC=C(c2ccc(S(=O)(=O)NCc3ccncc3)cc2)CC1 ; O=S(=O)(NCc1ccncc1)c1ccc(C2=CCC(F)(F)CC2)cc1 ; O=S(=O)(NCc1ccncc1)c1ccc(C2CCC(F)(F)CC2)cc1 ; O=S(=O)(NCc1ccncc1)c1ccc(C2CCOCC2)cc1 ; O=S(=O)(NCc1ccncc1)c1ccc(C2CCCOC2)cc1 ; O=S1(=O)CCC(c2ccc(S(=O)(=O)NCc3ccncc3)cc2)CC1]

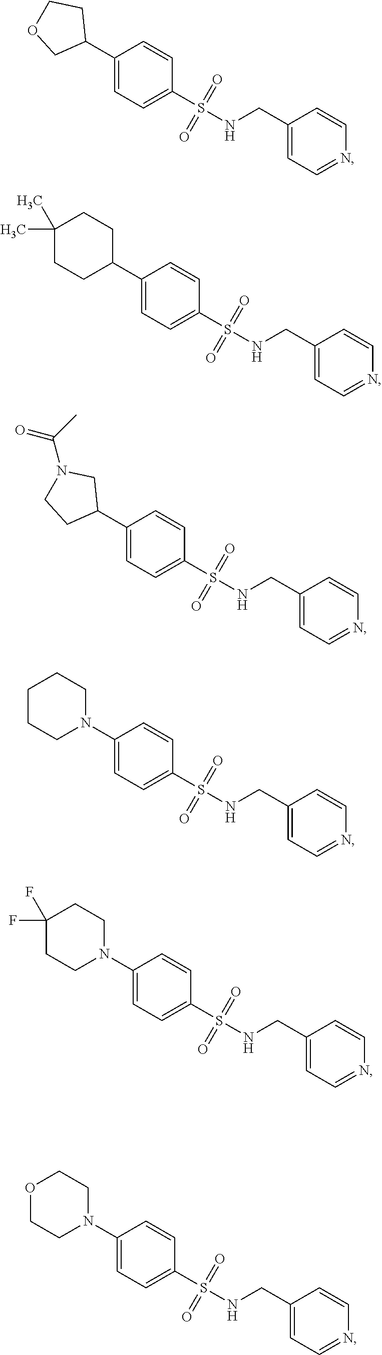 [SMILES: O=S(=O)(NCc1ccncc1)c1ccc(C2CCOC2)cc1 ; CC1(C)CCC(c2ccc(S(=O)(=O)NCc3ccncc3)cc2)CC1 ; CC(=O)N1CCC(c2ccc(S(=O)(=O)NCc3ccncc3)cc2)C1 ; O=S(=O)(NCc1ccncc1)c1ccc(N2CCCCC2)cc1 ; O=S(=O)(NCc1ccncc1)c1ccc(N2CCC(F)(F)CC2)cc1 ; O=S(=O)(NCc1ccncc1)c1ccc(N2CCOCC2)cc1]

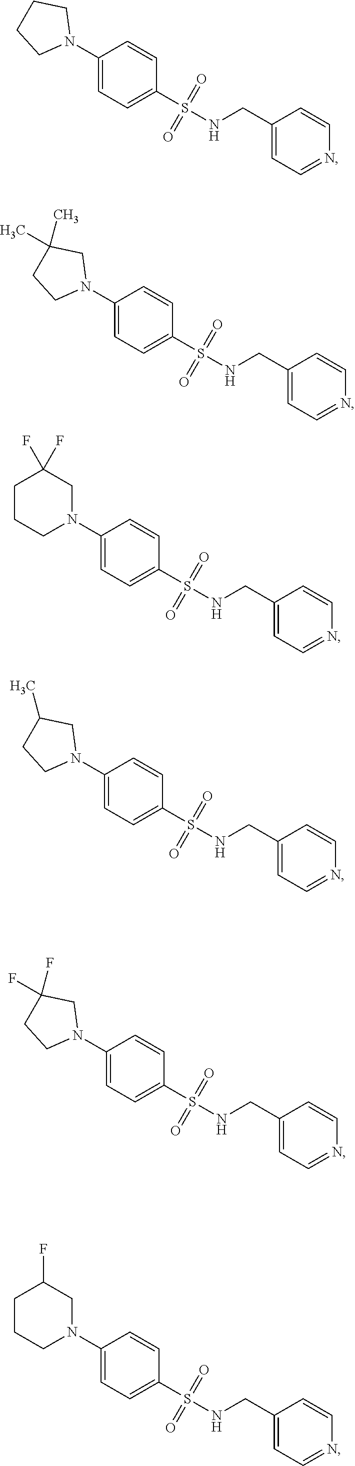 [SMILES: O=S(=O)(NCc1ccncc1)c1ccc(N2CCCC2)cc1 ; CC1(C)CCN(c2ccc(S(=O)(=O)NCc3ccncc3)cc2)C1 ; O=S(=O)(NCc1ccncc1)c1ccc(N2CCCC(F)(F)C2)cc1 ; CC1CCN(c2ccc(S(=O)(=O)NCc3ccncc3)cc2)C1 ; O=S(=O)(NCc1ccncc1)c1ccc(N2CCC(F)(F)C2)cc1 ; O=S(=O)(NCc1ccncc1)c1ccc(N2CCCC(F)C2)cc1]

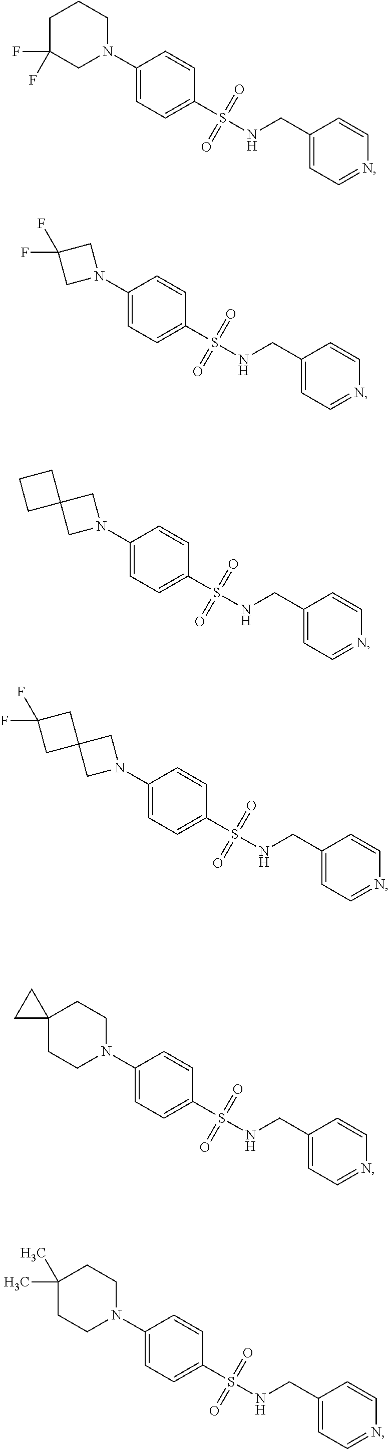 [SMILES: O=S(=O)(NCc1ccncc1)c1ccc(N2CCCC(F)(F)C2)cc1 ; O=S(=O)(NCc1ccncc1)c1ccc(N2CC(F)(F)C2)cc1 ; O=S(=O)(NCc1ccncc1)c1ccc(N2CC3(CCC3)C2)cc1 ; O=S(=O)(NCc1ccncc1)c1ccc(N2CC3(C2)CC(F)(F)C3)cc1 ; O=S(=O)(NCc1ccncc1)c1ccc(N2CCC3(CC2)CC3)cc1 ; CC1(C)CCN(c2ccc(S(=O)(=O)NCc3ccncc3)cc2)CC1]

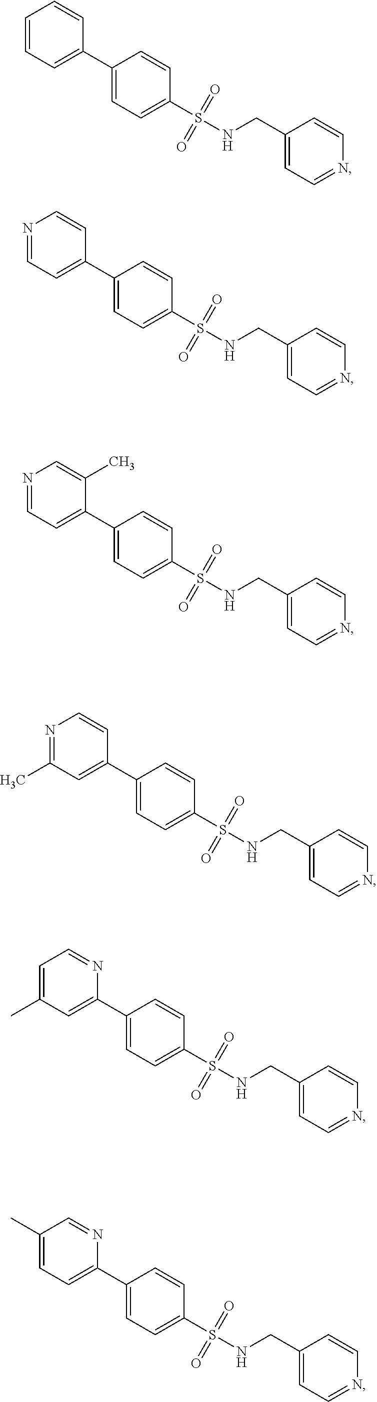 [SMILES: O=S(=O)(NCc1ccncc1)c1ccc(-c2ccccc2)cc1 ; O=S(=O)(NCc1ccncc1)c1ccc(-c2ccncc2)cc1 ; Cc1cnccc1-c1ccc(S(=O)(=O)NCc2ccncc2)cc1 ; Cc1cc(-c2ccc(S(=O)(=O)NCc3ccncc3)cc2)ccn1 ; Cc1ccnc(-c2ccc(S(=O)(=O)NCc3ccncc3)cc2)c1 ; Cc1ccc(-c2ccc(S(=O)(=O)NCc3ccncc3)cc2)nc1]

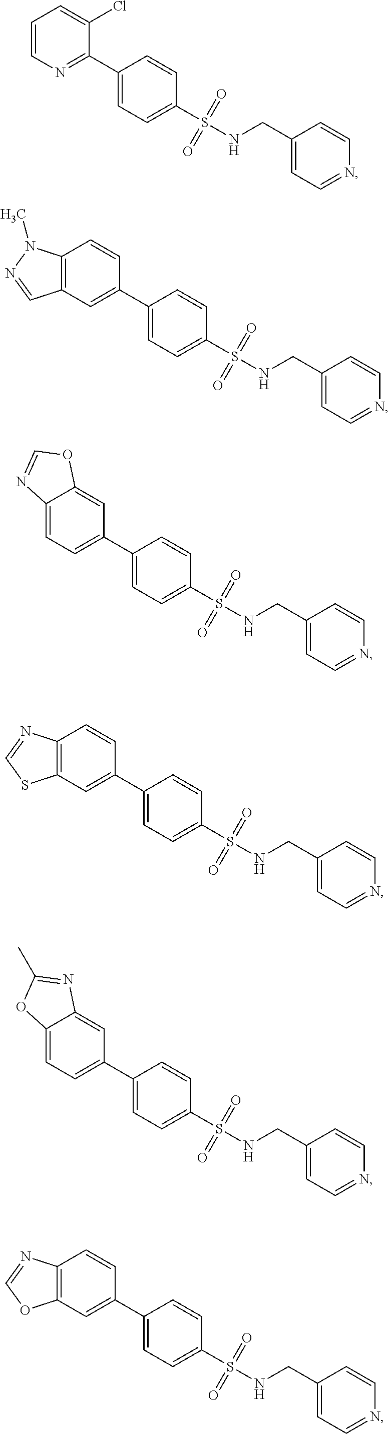 [SMILES: O=S(=O)(NCc1ccncc1)c1ccc(-c2ncccc2Cl)cc1 ; Cn1ncc2cc(-c3ccc(S(=O)(=O)NCc4ccncc4)cc3)ccc21 ; O=S(=O)(NCc1ccncc1)c1ccc(-c2ccc3ncoc3c2)cc1 ; O=S(=O)(NCc1ccncc1)c1ccc(-c2ccc3ncsc3c2)cc1 ; Cc1nc2cc(-c3ccc(S(=O)(=O)NCc4ccncc4)cc3)ccc2o1 ; O=S(=O)(NCc1ccncc1)c1ccc(-c2ccc3ncoc3c2)cc1]

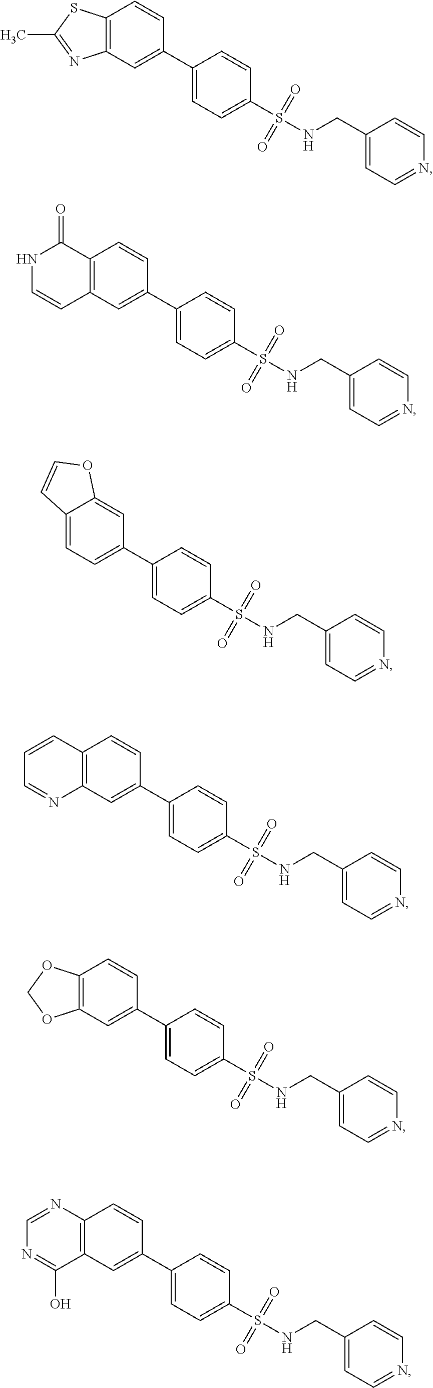 [SMILES: Cc1nc2cc(-c3ccc(S(=O)(=O)NCc4ccncc4)cc3)ccc2s1 ; O=c1[nH]ccc2cc(-c3ccc(S(=O)(=O)NCc4ccncc4)cc3)ccc12 ; O=S(=O)(NCc1ccncc1)c1ccc(-c2ccc3ccoc3c2)cc1 ; O=S(=O)(NCc1ccncc1)c1ccc(-c2ccc3cccnc3c2)cc1 ; O=S(=O)(NCc1ccncc1)c1ccc(-c2ccc3c(c2)OCO3)cc1 ; O=S(=O)(NCc1ccncc1)c1ccc(-c2ccc3ncnc(O)c3c2)cc1]

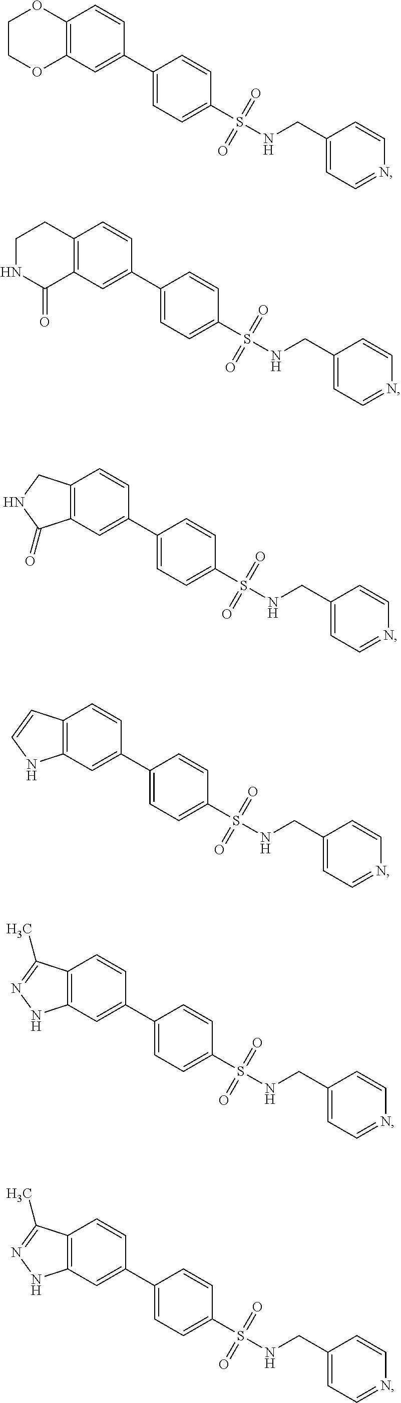 [SMILES: O=S(=O)(NCc1ccncc1)c1ccc(-c2ccc3c(c2)OCCO3)cc1 ; O=C1NCCc2ccc(-c3ccc(S(=O)(=O)NCc4ccncc4)cc3)cc21 ; O=C1NCc2ccc(-c3ccc(S(=O)(=O)NCc4ccncc4)cc3)cc21 ; O=S(=O)(NCc1ccncc1)c1ccc(-c2ccc3cc[nH]c3c2)cc1 ; Cc1n[nH]c2cc(-c3ccc(S(=O)(=O)NCc4ccncc4)cc3)ccc12 ; Cc1n[nH]c2cc(-c3ccc(S(=O)(=O)NCc4ccncc4)cc3)ccc12]

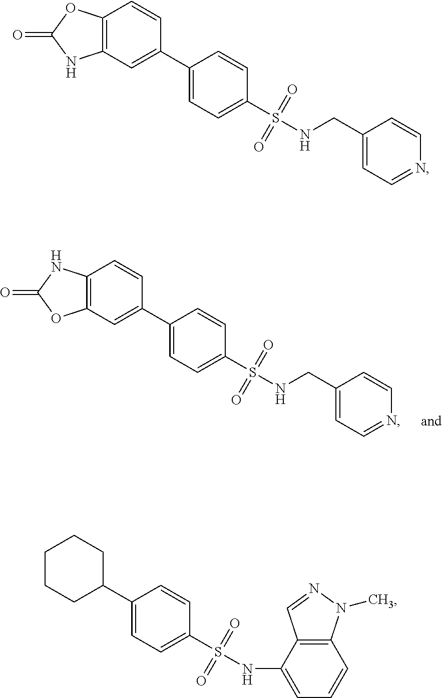 [SMILES: O=c1[nH]c2cc(-c3ccc(S(=O)(=O)NCc4ccncc4)cc3)ccc2o1 ; O=c1[nH]c2ccc(-c3ccc(S(=O)(=O)NCc4ccncc4)cc3)cc2o1 ; Cn1ncc2c(NS(=O)(=O)c3ccc(C4CCCCC4)cc3)cccc21]

or a pharmaceutically acceptable salt thereof.

Also provided is a compound chosen from the Examples disclosed herein, or a pharmaceutically acceptable salt thereof.

The compounds disclosed herein can exist as pharmaceutically acceptable salts. The present disclosure includes compounds listed herein in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “pharmaceutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate, L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form pharmaceutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present disclosure contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of pharmaceutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine, N,N-dibenzylphenethylamine, 1-ephenamine, and N,N-dibenzylethylenediamine Other representative organic amines useful for the formation of base addition salts include ethylenediamine, ethanolamine, diethanolamine, piperidine, and piperazine.

While it may be possible for the compounds, and pharmaceutically acceptable salts thereof, of the subject disclosure to be administered as the raw chemical, it is also possible to present them as a pharmaceutical formulation.

Also provided is a pharmaceutical formulation comprising a compound as disclosed herein, or a pharmaceutically acceptable salt thereof, together with a pharmaceutically acceptable carrier.

In some embodiments, the pharmaceutical formulation is formulated for oral administration.

In some embodiments, the oral pharmaceutical formulation is chosen from a tablet and a capsule.

The formulations include those suitable for oral, parenteral (including subcutaneous, intradermal, intramuscular, intravenous, intraarticular, and intramedullary), intraperitoneal, transmucosal, transdermal, rectal and topical (including dermal, buccal, sublingual and intraocular) administration although the most suitable route may depend upon for example the condition and disorder of the recipient. The formulations may conveniently be presented in unit dosage form and may be prepared by any of the methods well known in the art of pharmacy. Typically, these methods include the step of bringing into association a compound, or pharmaceutically acceptable salts thereof, of the subject disclosure or a pharmaceutically acceptable salt thereof (“active ingredient”) with the carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations of the compounds, or pharmaceutically acceptable salts thereof disclosed herein suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste.

Pharmaceutical formulations which can be used orally include tablets, push-fit capsules made of gelatin, as well as soft, sealed capsules made of gelatin and a plasticizer, such as glycerol or sorbitol. Tablets may be made by compression or molding, optionally with one or more accessory ingredients. Compressed tablets may be prepared by compressing in a suitable machine the active ingredient in a free-flowing form such as a powder or granules, optionally mixed with binders, inert diluents, or lubricating, surface active or dispersing agents. Molded tablets may be made by molding in a suitable machine a mixture of the powdered compound, or a pharmaceutically acceptable salt thereof, moistened with an inert liquid diluent. The tablets may optionally be coated or scored and may be formulated so as to provide slow or controlled release of the active ingredient therein. All formulations for oral administration should be in dosages suitable for such administration. The push-fit capsules can contain the active ingredients in admixture with filler such as lactose, binders such as starches, and/or lubricants such as talc or magnesium stearate and, optionally, stabilizers. In soft capsules, the active compounds, or pharmaceutically acceptable salts thereof, may be dissolved or suspended in suitable liquids, such as fatty oils, liquid paraffin, or liquid polyethylene glycols. In addition, stabilizers may be added. Dragee cores are provided with suitable coatings. For this purpose, concentrated sugar solutions may be used, which may optionally contain gum arabic, talc, polyvinyl pyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses, or pharmaceutically acceptable salts thereof.

The compounds, or a pharmaceutically acceptable salt thereof, may be formulated for parenteral administration by injection, e.g., by bolus injection or continuous infusion. Formulations for injection may be presented in unit dosage form, e.g., in ampoules or in multi-dose containers, with an added preservative. The formulations may take such forms as suspensions, solutions or emulsions in oily or aqueous vehicles, and may contain formulatory agents such as suspending, stabilizing and/or dispersing agents. The formulations may be presented in unit-dose or multi-dose containers, for example sealed ampoules and vials, and may be stored in powder form or in a freeze-dried (lyophilized) condition requiring only the addition of the sterile liquid carrier, for example, saline or sterile pyrogen-free water, immediately prior to use. Extemporaneous injection solutions and suspensions may be prepared from sterile powders, granules and tablets of the kind previously described.

Formulations for parenteral administration include aqueous and non-aqueous (oily) sterile injection solutions of the active compounds, or a pharmaceutically acceptable salt thereof, which may contain antioxidants, buffers, bacteriostats and solutes which render the formulation isotonic with the blood of the intended recipient; and aqueous and non-aqueous sterile suspensions which may include suspending agents and thickening agents. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds, or pharmaceutically acceptable salts thereof, to allow for the preparation of highly concentrated solutions.

In addition to the formulations described previously, the compounds, or pharmaceutically acceptable salts thereof, may also be formulated as a depot preparation. Such long acting formulations may be administered by implantation (for example subcutaneously or intramuscularly) or by intramuscular injection. Thus, for example, the compounds, or pharmaceutically acceptable salts thereof, may be formulated with suitable polymeric or hydrophobic materials (for example as an emulsion in an acceptable oil) or ion exchange resins, or as sparingly soluble derivatives, for example, as a sparingly soluble salt.

For buccal or sublingual administration, the formulations may take the form of tablets, lozenges, pastilles, or gels formulated in conventional manner Such formulations may comprise the active ingredient in a flavored basis such as sucrose and acacia or tragacanth.

The compounds, or pharmaceutically acceptable salts thereof, may also be formulated in rectal formulations such as suppositories or retention enemas, e.g., containing conventional suppository bases such as cocoa butter, polyethylene glycol, or other glycerides.

Certain compounds, or pharmaceutically acceptable salts thereof, disclosed herein may be administered topically, that is by non-systemic administration. This includes the application of a compound, or a pharmaceutically acceptable salt thereof, disclosed herein externally to the epidermis or the buccal cavity and the instillation of such a compound, or a pharmaceutically acceptable salt thereof, into the ear, eye and nose, such that the compound (or pharmaceutically acceptable salt thereof) does not significantly enter the blood stream. In contrast, systemic administration refers to oral, intravenous, intraperitoneal and intramuscular administration.

Formulations suitable for topical administration include liquid or semi-liquid preparations suitable for penetration through the skin to the site of inflammation such as gels, liniments, lotions, creams, ointments or pastes, and drops suitable for administration to the eye, ear or nose. The active ingredient for topical administration may comprise, for example, from 0.001% to 10% w/w (by weight) of the formulation. In some embodiments, the active ingredient may comprise as much as 10% w/w. In some embodiments, it may comprise less than 5% w/w. In some embodiments, the active ingredient may comprise from 2% w/w to 5% w/w. In some embodiments, it may comprise from 0.1% to 1% w/w of the formulation.

For administration by inhalation, compounds, or pharmaceutically acceptable salts thereof, may be conveniently delivered from an insufflator, nebulizer pressurized packs or other convenient means of delivering an aerosol spray. Pressurized packs may comprise a suitable propellant such as dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Alternatively, for administration by inhalation or insufflation, the compounds, and pharmaceutically acceptable salts thereof, according to the disclosure may take the form of a dry powder formulation, for example a powder mix of the compound, or pharmaceutically acceptable salt thereof, and a suitable powder base such as lactose or starch. The powder formulation may be presented in unit dosage form, in for example, capsules, cartridges, gelatin or blister packs from which the powder may be administered with the aid of an inhalator or insufflator.

Preferred unit dosage formulations are those containing an effective dose, or an appropriate fraction thereof, of the active ingredient.

It should be understood that in addition to the ingredients particularly mentioned herein, the formulations described herein may include other agents conventional in the art having regard to the type of formulation in question, for example those suitable for oral administration may include flavoring agents.

Also provided are methods of inhibiting at least one PU.1 function comprising the step of contacting PU.1 with a compound as described herein, or a pharmaceutically acceptable salt thereof. The cell phenotype, cell proliferation, activity of PU.1, change in biochemical output produced by active PU.1, expression of PU.1, or binding of PU.1 with a natural binding partner may be monitored. Such methods may be modes of treatment of disease, biological assays, cellular assays, biochemical assays, or the like.

Also provided herein are methods of treatment of a PU.1-mediated disease comprising the administration of a therapeutically effective amount of a compound as disclosed herein, or a pharmaceutically acceptable salt thereof, to a patient in need thereof.

Also provided herein are methods of treatment of an inflammatory component of an PU.1-mediated disease.

Also provided herein is a method of inhibition of PU.1 comprising contacting PU.1 with a compound as disclosed herein, or a pharmaceutically acceptable salt thereof.

Also provided is a method of modulation of a PU.1-mediated function in a subject comprising the administration of a therapeutically effective amount of a compound as disclosed herein, or a pharmaceutically acceptable salt thereof.

In some embodiments, the disorder is a disorder related to, or affected by, PU.1 expression or repression. In some embodiments, the disorder is related to abnormal PU.1 expression. In some embodiments, the disorder is chosen from multiple sclerosis, Parkinson's Disease, Huntington's Disease, amyotrophic lateral sclerosis, neuroinflammation, frontotemporal dementia, dementia with Lewy bodies, neuropathic pain, inflammatory pain, neuropathic itch, inflammatory itch, neuropathic dysesthesia, inflammatory dysesthesia, dementia, glioma, brain tumors, Batten disease, Down's Syndrome, Nasu-Hakola, prion disease, Cockayne syndrome, Ataxia-telangiectasia, xeroderma pigmentosum, schizophrenia, bipolar disorder, epilepsy, motor neuron disease, sciatica, Friedreich's ataxia, Gerstmann-Straussler-Scheinker Disease, Kuru, Alper's Disease, apnea, corticobasal degeneration, Leigh's Disease, Monomelic amyotrophy, multiple system atrophy, multiple system atrophy with orthostatic hypotension, narcolepsy, neurodegeneration with brain iron accumulation, opsoclonus myoclonus, progressive multifocal leukoencephalopathy, strationigral degeneration, transmissible spongiform encephalopathis, ataxia, Sjogren's disease, Sandhoff disease, Myasthenia gravis, Tay-Sachs disease, neuronal ceroid lipofuscinosis, senesence, progeria, sepsis, Lyme disease, leukemia, lupus, fibrosis, cancer, hematologic cancer, bone cancer, glioblastomas, inflammatory diseases, inflammatory disorders, autoimmune disorders, endotoxemia and neurodegenerative diseases, including without limitation, such conditions ase acute myeloid leukemia, rheumatoid arthritis, contact dermatitis, asthma, inflammatory bowel disease, pediatric atrophy, giant cell arteritis, Alzheimer's disease, and systemic lupus. In some embodiments, the disorder is a disorder related to cells from a hematopoietic lineage.

In some embodiments, the compounds, pharmaceutically acceptable salts, formulations, and methods disclosed herein may be useful for the treatment of a disorder chosen from Alzheimer's disease, inflammation, or excessive myelin uptake.

In some embodiments, the compounds, pharmaceutically acceptable salts, formulations, and methods disclosed herein may be useful for the treatment of Alzheimer's disease.

In some embodiments, the compounds, pharmaceutically acceptable salts formulations, and methods disclosed herein may be coadministered with another therapeutic agent.

A PU.1 inhibitor may be optimally used together with an amyloid antibody or a cognitive enhancer.

Compounds, or pharmaceutically acceptable salts thereof, may be administered orally or via injection at a dose of from 0.1 to 500 mg/kg per day. The dose range for adult humans is generally from 5 mg to 2 g/day. Tablets or other forms of presentation provided in discrete units may conveniently contain an amount of one or more compounds, or pharmaceutically acceptable salts thereof, which is effective at such dosage or as a multiple of the same, for instance, units containing 5 mg to 500 mg, usually around 10 mg to 200 mg.

The amount of active ingredient that may be combined with the carrier materials to produce a single dosage form will vary depending upon the host treated and the particular mode of administration.

The compounds, or pharmaceutically acceptable salts thereof, can be administered in various modes, e.g. orally, topically, or by injection. The precise amount of compound, or pharmaceutically acceptable salt thereof, administered to a patient will be the responsibility of the attendant physician. The specific dose level for any particular patient will depend upon a variety of factors including the activity of the specific compound employed, or pharmaceutically acceptable salt thereof, the age, body weight, general health, sex, diets, time of administration, route of administration, rate of excretion, drug combination, the precise disorder being treated, and the severity of the indication or condition being treated. Also, the route of administration may vary depending on the condition and its severity.

In certain instances, it may be appropriate to administer at least one of the compounds described herein (or a pharmaceutically acceptable salt thereof) in combination with another therapeutic agent. By way of example only, if one of the side effects experienced by a patient upon receiving one of the compounds herein, or pharmaceutically acceptable salt thereof, is hypertension, then it may be appropriate to administer an anti-hypertensive agent in combination with the initial therapeutic agent. Or, by way of example only, the therapeutic effectiveness of one of the compounds described herein, or pharmaceutically acceptable salts thereof, may be enhanced by administration of an adjuvant (i.e., by itself the adjuvant may only have minimal therapeutic benefit, but in combination with another therapeutic agent, the overall therapeutic benefit to the patient is enhanced). Or, by way of example only, the benefit of experienced by a patient may be increased by administering one of the compounds described herein, or pharmaceutically acceptable salts thereof, with another therapeutic agent (which also includes a therapeutic regimen) that also has therapeutic benefit. By way of example only, in a treatment for diabetes involving administration of one of the compounds described herein, or pharmaceutically acceptable salts thereof, increased therapeutic benefit may result by also providing the patient with another therapeutic agent for diabetes. In any case, regardless of the disease, disorder or condition being treated, the overall benefit experienced by the patient may simply be additive of the two therapeutic agents or the patient may experience a synergistic benefit.

Specific, non-limiting examples of possible combination therapies include use of certain compounds and pharmaceutically acceptable salts of the disclosure with: donepezil, rivastigmine, galantamine, and memantine. Further examples include anti-amyloid antibodies and vaccines, anti-Ab antibodies and vaccines, anti-tau antibodies and vaccines, β-secretase inhibitors, 5-HT4 agonists, 5-HT6 antagonists, 5-HT1a antagonists, α7 nicotinic receptor agonists, 5-HT3 receptor antagonists, PDE4 inhibitors, O-glycnacase inhibitors, and other medicines approved for the treatment of Alzheimer's disease. Further examples include metformin, minocycline, tissue plasminogen activator, and other therapies that improve neuronal survival.

In any case, the multiple therapeutic agents (at least one of which is a compound disclosed herein, or a pharmaceutically acceptable salt thereof) may be administered in any order or even simultaneously. If simultaneously, the multiple therapeutic agents may be provided in a single, unified form, or in multiple forms (by way of example only, either as a single pill or as two separate pills). One of the therapeutic agents may be given in multiple doses, or both may be given as multiple doses. If not simultaneous, the timing between the multiple doses may be any duration of time ranging from a few minutes to four weeks.

Further embodiments include the embodiments disclosed in the following Examples, which is not to be construed as limiting in any way.

### Example 1

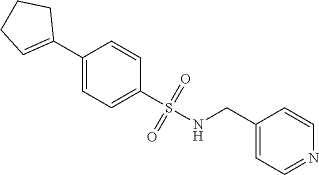 [SMILES: O=S(=O)(NCc1ccncc1)c1ccc(C2=CCCC2)cc1]

### 4-(Cyclopent-1-en-1-yl)-N-(pyridin-4-ylmethyl)benzenesulfonamide

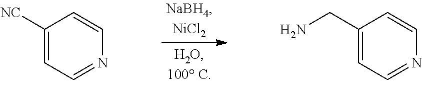 [SMILES: N#Cc1ccncc1 ; NCc1ccncc1]

Pyridin-4-ylmethanamine A mixture of isonicotinonitrile (2.1 g, 10 mmol), NaBH₄ (380 mg, 10 mmol) and NiCl₂ (1.3 g, 10 mmol) in water (20 mL) was stirred at 100° C. for 8 hr. The mixture was cooled to rt, filtered, and the filtrate was concentrated to afford the title compound as a brown solid (1.89 g, 88%). MS (ES+) C₆H₈N₂ requires: 108, found: 109[M+H]⁺.

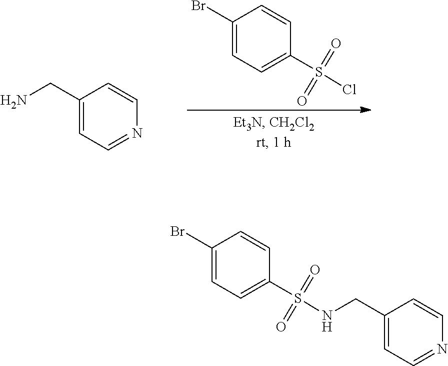 [SMILES: NCc1ccncc1 ; O=S(=O)(Cl)c1ccc(Br)cc1 ; O=S(=O)(NCc1ccncc1)c1ccc(Br)cc1]

4-Bromo-N-(pyridin-4-ylmethyl)benzenesulfonamide A mixture of the product from the previous step (1.9 g, 17 mmol), 4-bromobenzenesulfonyl chloride (4.5 g, 17 mmol) and Et₃N (3.5 g, 35 mmol) in CH₂Cl₂ (20 mL) was stirred at rt for 1 hr. The solvent was removed under reduced pressure and the residue was purified by silica gel chromatography (EtOAc=100%) to afford the title compound as a yellow solid (2.5 g, 44%). MS (ES+) C₁₂H₁₁BrN₂O₂S requires: 326, found: 327[M+H]⁺.

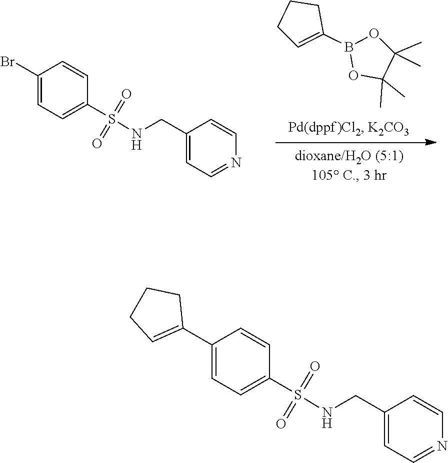 [SMILES: O=S(=O)(NCc1ccncc1)c1ccc(Br)cc1 ; CC1(C)OB(C2=CCCC2)OC1(C)C ; O=S(=O)(NCc1ccncc1)c1ccc(C2=CCCC2)cc1]

4-(Cyclopent-1-en-1-yl)-N-(pyridin-4-ylmethyl)benzenesulfonamide A mixture of the product from the previous step, 2-cyclopenten-1-yl-4,4,5,5-tetramethyl-1,3,2-dioxaborolane (59 mg, 0.3 mmol), Pd(dppf)Cl₂ (22 mg, 0.03 mmol), K₂CO₃ (124 mg, 0.9 mmol) in dioxane (10 mL) and water (2 mL) was stirred at 105° C. for 3 h. The mixture was purified by prep-HPLC (0.1% TFA in H₂O:CH₃CN=10-50%, C18) to afford the title compound as a white solid (19 mg, 20%).

MS (ES+) C₁₇H₁₈N₂O₂S requires: 314, found: 315[M+H]⁺.

¹HNMR (500 MHz, CD₃OD) δ 8.42-8.39 (m, 2H), 7.81-7.80 (m, 2H), 7.61-7.60 (m, 2H), 7.34-7.33 (m, 2H), 6.44 (m, 1H), 4.17 (s, 2H), 2.76-2.75 (m, 2H), 2.60-2.59 (m, 2H), 2.10-2.05 (m, 2H).

### Example 2

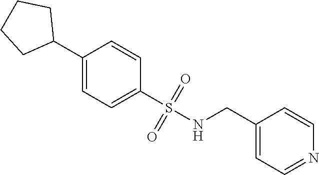 [SMILES: O=S(=O)(NCc1ccncc1)c1ccc(C2CCCC2)cc1]

### 4-cyclopentyl-N-(pyridin-4-ylmethyl)benzenesulfonamide

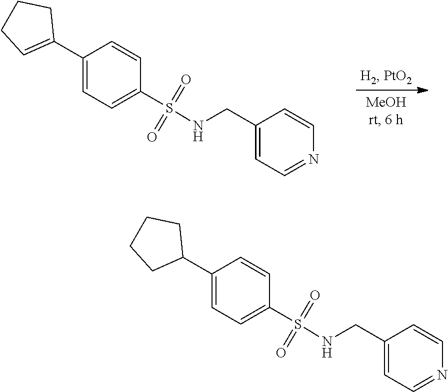 [SMILES: O=S(=O)(NCc1ccncc1)c1ccc(C2=CCCC2)cc1 ; O=S(=O)(NCc1ccncc1)c1ccc(C2CCCC2)cc1]

A mixture of the Example 1 compound (55 mg, 0.17 mmol) and PtO₂ (10 mg, cat.) in MeOH (6 mL) was stirred at rt for 6 hr under hydrogen. The reaction mixture was filtered, the solvent was removed under reduced pressure, and the residue was purified by prep-HPLC (0.1% TFA in H₂O:CH₃CN=10-50%, C18) to afford the title compound as a white solid (31 mg, 56%).

MS (ES+) C₁₇H₂₀N₂O₂S requires: 316, found: 317[M+H]⁺.

¹HNMR (500 MHz, CD₃OD) δ 8.42-8.39 (m, 2H), 7.81-7.80 (m, 2H), 7.61-7.60 (m, 2H), 7.34-7.33 (m, 2H), 4.17 (s, 2H), 3.08-3.06 (m, 1H), 2.76-2.75 (m, 2H), 2.60-2.59 (m, 2H), 2.10-2.05 (m, 2H).

### Example 3

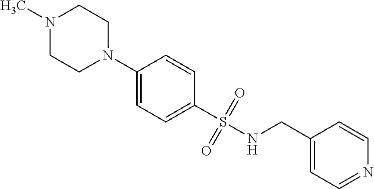 [SMILES: CN1CCN(c2ccc(S(=O)(=O)NCc3ccncc3)cc2)CC1]

### 4-(4-methylpiperazin-1-yl)-N-(pyridin-4-ylmethyl)benzenesulfonamide

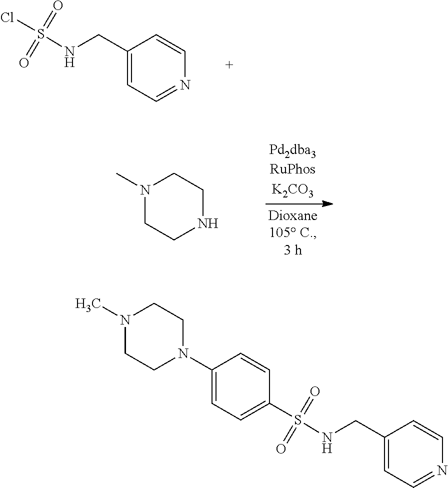

A mixture of 4-bromo-N-(pyridin-4-ylmethyl)benzenesulfonamide (0.098 g, 0.3 mmol), 1-methylpiperazine (0.03 g, 0.3 mmol), Pd₂dba₃ (0.027 g, 0.03 mmol), Cs₂CO₃ (0.29 g, 0.9 mmol), RuPhos (0.013 g, 0.03 mmol) in dioxane (4 mL) was stirred at 105° C. for 3 h. The mixture was filtered through CELITE®, and the solvent was removed under reduced pressure. The residue was purified by prep-HPLC (0.1% TFA in H₂O:CH₃CN=10-50%, C18) to afford the title compound as a white solid (6 mg, 6%).

MS (ES+) C₁₇H₂₂N₄O₂S requires: 346, found: 347[M+H]⁺.

¹HNMR (400 MHz, CD₃OD) δ 8.42-8.40 (m, 2H), 7.66-7.63 (m, 2H), 7.34-7.32 (m, 2H), 7.02-6.98 (m, 2H), 4.11 (s, 2H), 3.37-3.33 (m, 4H), 2.62-2.60 (m, 4H), 1.37 (s, 3H).

### Example 4

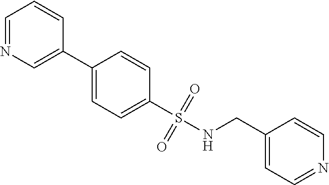 [SMILES: O=S(=O)(NCc1ccncc1)c1ccc(-c2cccnc2)cc1]

### 4-(pyridin-3-yl)-N-(pyridin-4-ylmethyl)benzenesulfonamide

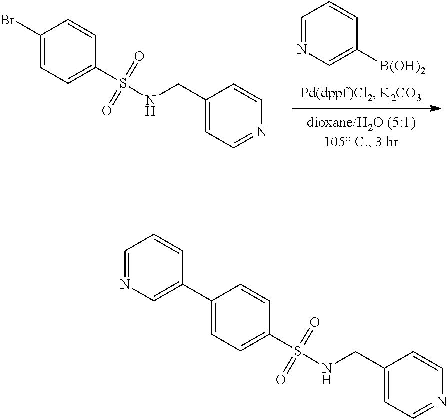 [SMILES: O=S(=O)(NCc1ccncc1)c1ccc(Br)cc1 ; OB(O)c1cccnc1 ; O=S(=O)(NCc1ccncc1)c1ccc(-c2cccnc2)cc1]

A mixture of 4-bromo-N-(pyridin-4-ylmethyl)benzenesulfonamide (100 mg, 0.305 mmol), pyridin-3-ylboronic acid (75 mg, 0.609 mmol), Pd(dppf)Cl₂ (22 mg, 0.0305 mmol), K₂CO₃ (124 mg, 0.915 mmol) in dioxane (10 mL) and water (2 mL) was stirred at 105° C. for 3 hr. TFA (3 ml) was added and the mixture was stirred at rt for 3 hr. The solids were removed by filtration and the filtrate was evaporated under reduced pressure. The residue was purified by prep-HPLC (0.1% TFA in H₂O:CH₃CN=10-50%, C18) to afford 21 mg (22%) of the title compound as a white solid.

MS (ES+) C₁₇H₁₅N₃O₂S requires: 325, found: 326[M+H]⁺.

¹HNMR (40 0 MHz, CD₃OD) δ 8.70-8.69 (m, 2H), 8.45-8.43 (m, 2H), 7.98-7.75 (m, 6H), 7.27-7.26 (m, 2H), 4.06 (s, 2H).

### Example 5

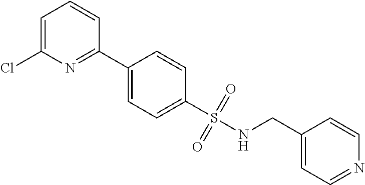 [SMILES: O=S(=O)(NCc1ccncc1)c1ccc(-c2cccc(Cl)n2)cc1]

### 4-(6-chloropyridin-2-yl)-N-(pyridin-4-ylmethyl)benzenesulfonamide

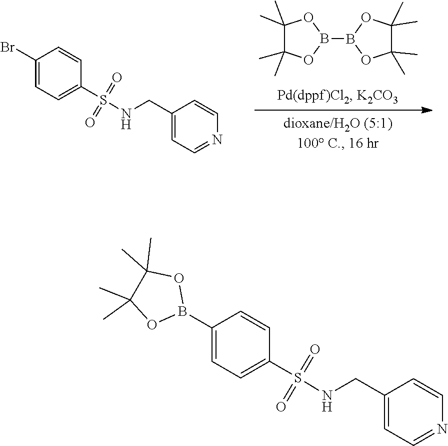

N-(pyridin-4-ylmethyl)-4-(4,4,5,5-tetramethyl-1,3,2-dioxaborolan-2-yl)benzenesulfonamide A mixture of 4-bromo-N-(pyridin-4-ylmethyl)benzenesulfonamide (2 g, 6.1 mmol), B₂pin₂ (1.86 g, 7.3 mmol), KOAc (1.18 g, 12.2 mmol) and Pd(dppf)Cl₂ (223 mg, 0.3 mmol) in dioxane (25 mL) was stirred at 100° for 16 hr, diluted with EtOAc, and washed with brine. The solvent was removed under reduced pressure to give the crude title product which was used directly in next step (2.2 g, 96%). MS (ES+) C₁₈H₂₃BN₂O₄S requires: 374, found: 375[M+H]⁺.

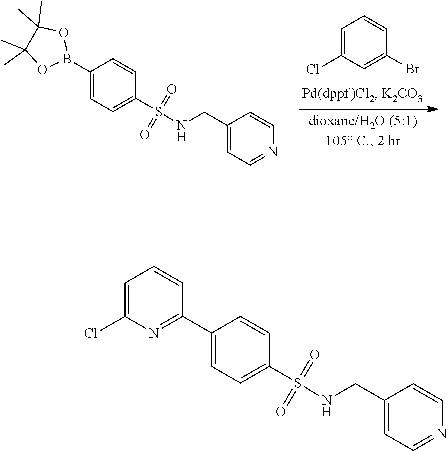 [SMILES: CC1(C)OB(c2ccc(S(=O)(=O)NCc3ccncc3)cc2)OC1(C)C ; Clc1cccc(Br)c1 ; O=S(=O)(NCc1ccncc1)c1ccc(-c2cccc(Cl)n2)cc1]

4-(6-chloropyridin-2-yl)-N-(pyridin-4-ylmethyl)benzenesulfonamide A solution of the product from the previous step (100 mg, 0.267 mmol), 2-bromo-6-chloropyridine (103 mg, 0.534 mmol), K₂CO₃ (111 mg, 0.801 mmol), and Pd(dppf)Cl₂ (10 mg, 0.013 mmol) in dioxane:water 5:1 (6 mL) was refluxed at 105° C. for 2 hr. The reaction mixture was filtered, concentrated under reduced pressure, and purified by prep-HPLC to afford the title compound (18.5 mg, 19%) as a white solid.

MS (ES+) C₁₇H₁₄ClN₃O₂S requires: 359, found: 360 [M+H]⁺.

¹H NMR (400 MHz, DMSO-d₆) δ 8.49-8.40 (m, 3H), 8.25 (d, J=8.6 Hz, 2H), 8.10 (d, J=7.1 Hz, 1H), 8.03 (t, J=7.8 Hz, 1H), 7.92 (d, J=8.6 Hz, 2H), 7.58 (d, J=7.8 Hz, 1H), 7.27 (d, J=6.0 Hz, 2H), 4.09 (d, J=6.3 Hz, 2H).

### Example 6

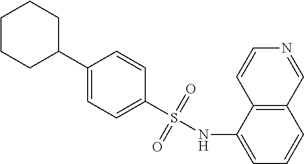 [SMILES: O=S(=O)(Nc1cccc2cnccc12)c1ccc(C2CCCCC2)cc1]

### 4-Cyclohexyl-N-(isoquinolin-5-yl)benzenesulfonamide

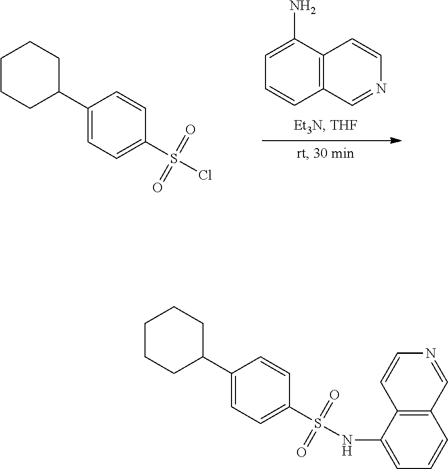 [SMILES: O=S(=O)(Cl)c1ccc(C2CCCCC2)cc1 ; Nc1cccc2cnccc12 ; O=S(=O)(Nc1cccc2cnccc12)c1ccc(C2CCCCC2)cc1]

A mixture of 4-cyclohexylbenzenesulfonyl chloride (129 mg, 0.5 mmol), isoquinolin-5-amine (72 mg, 0.5 mmol) and Et₃N (151 mg, 1.5 mmol) in THF (3 mL) was stirred at rt for 30 min. H₂O (20 mL) was added and the mixture was extracted with EtOAc (20 ml). The combined organic layers were concentrated and purified by prep-HPLC (0.1% TFA in H₂O:CH₃CN=10-50%, C18) to afford 5 mg (3%) of the title compound as a white solid.

MS (ES+) C₂₁H₂₂N₂O₂S requires: 366, found: 367[M+H]⁺.

¹H NMR (400 MHz, CD₃OD) δ 9.15 (s, 1H), 8.27-8.25 (s, 1H), 7.89-7.82 (m, 2H), 7.60-7.55 (m, 4H), 7.26-7.24 (m, 2H), 2.55-2.54 (m, 1H), 1.85-1.74 (m, 5H), 1.43-1.30 (m, 5H).

### Example 7

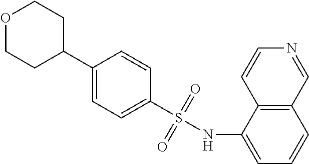 [SMILES: O=S(=O)(Nc1cccc2cnccc12)c1ccc(C2CCOCC2)cc1]

### N-(Isoquinolin-5-yl)-4-(tetrahydro-2H-pyran-4-yl)enzenesulfonamide

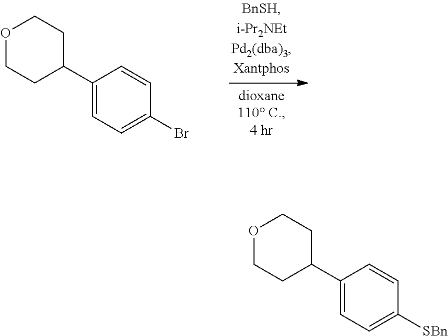

4-(4-(Benzylthio)phenyl)tetrahydro-2H-pyran A mixture of 4-(4-bromophenyl)tetrahydro-2H-pyran (200 mg, 0.83 mmol), benzyl mercaptan (207 mg, 1.67 mmol), i-Pr₂NEt (215 mg, 1.67 mmol), XantPhos (24 mg, 0.042 mmol) and Pd₂(dba)₃ (38 mg, 0.042 mmol) in dioxane (3 mL) was stirred at 110° C. for 4 hr. The mixture was filtered, the solvent was evaporated under reduced pressure, and the residue was purified by silica gel chromatography eluting with PE/EtOAc (30%) to afford the title compound as a yellow solid (180 mg, 76%). MS (ES+) C₁₈H₂₀OS requires: 284, found: 285 [M+H]⁺.

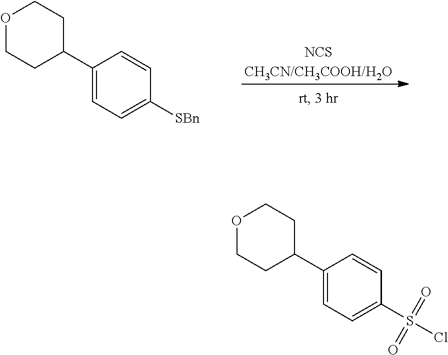

4-(Tetrahydro-2H-pyran-4-yl)benzenesulfonyl chloride A mixture of the product from the previous step (180 mg, 0.63 mmol) and N-chlorosuccinimide (168 mg, 1.27 mmol) in CH₃CN/HOAc/H₂O (3 mL/0.15 mL/0.15 mL) was stirred at rt for 3 hr. H₂O (20 mL) was added. The mixture was extracted with EtOAc, and the organic layer was washed with aq. NaHSO₃, dried and concentrated under reduced pressure to afford the title compound as a yellow oil (160 mg, crude). MS (ES+) C₁₁H₁₃ClO₃S requires: 260, found: 261 [M+H]⁺.

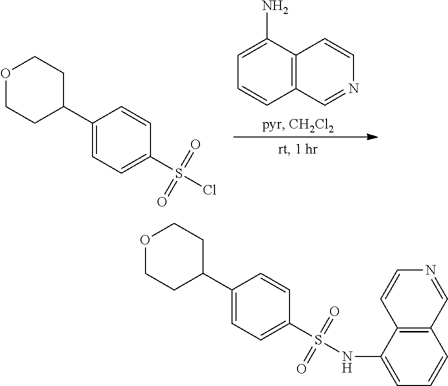

N-(Isoquinolin-5-yl)-4-(tetrahydro-2H-pyran-4-yl)benzenesulfonamide A mixture of the product from the previous step (70 mg crude, 0.27 mmol), isoquinolin-5-amine (58 mg, 0.40 mmol) and pyridine (43 mg, 0.54 mmol) in CH₂C₁₂ (2 mL) was stirred at rt for 1 hr. The mixture was concentrated and purified by prep-HPLC (NH₄HCO₃) to provide the title compound as a white solid (30 mg, 30%).

MS (ES+) C₂₀H₂₀N₂O₃S requires: 368, found: 369 [M+H]⁺.

¹H NMR (400 MHz, DMSO-d₆) δ 10.36 (s, 1H), 9.26 (s, 1H), 7.97 (d, J=8.2 Hz, 1H), 7.73 (d, J=5.7 Hz, 1H), 7.65-7.54 (m, 3H), 7.48 (d, J=7.4 Hz, 1H), 7.36 (d, J=8.3 Hz, 2H), 3.99-3.84 (m, 2H), 3.40 (td, J=11.1, 3.4 Hz, 2H), 2.87-2.74 (m, 1H), 1.69-1.50 (m, 4H).

### Example 8

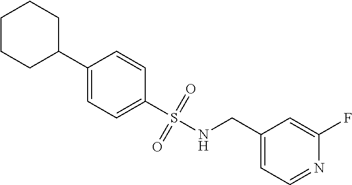 [SMILES: O=S(=O)(NCc1ccnc(F)c1)c1ccc(C2CCCCC2)cc1]

### 4-cyclohexyl-N-((2-fluoropyridin-4-yl)methyl)benzenesulfonamide

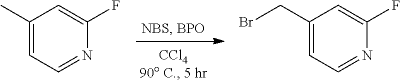

4-(Bromomethyl)-2-fluoropyridine To a solution of 2-fluoro-4-methylpyridine (500 mg, 4.505 mmol) in CCl₄ (10 mL) was added NBS (882 mg, 4.955 mmol) and BPO (164 mg, 0.676 mmol). The mixture was heated to 90° C. for 5 hr. Then the reaction was diluted with CH₂Cl₂, washed 3 times with water, dried over Na₂SO₄, filtered and concentrated in vacuo. The residue was purified via silica gel chromatography, eluting with 0-15% EtOAc in PE to afford the title compound (240 mg, 28%) as a green oil. MS (ES+) C₆H₅BrFN requires: 190, found: 191 [M+H]⁺.

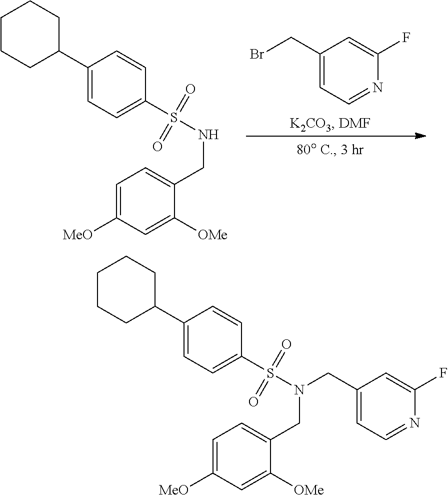

4-Cyclohexyl-N-(2,4-dimethoxybenzyl)-N-((2-fluoropyridin-4-yl)methyl)-benzenesulfonamide To a solution of the product from the previous step (50 mg, 0.263 mmol) in DMF (1 mL) was added 4-cyclohexyl-N-(2,4-dimethoxybenzyl)benzene-sulfonamide (154 mg, 0.395 mmol) and K₂CO₃ (73 mg, 0.526 mmol). The mixture was heated to 80° C. for 3 hr. LCMS showed the reaction was complete. The mixture was cooled, washed with water, extracted with EtOAc, washed with saturated brine, dried over Na₂SO₄, filtered and concentrated. The residue was purified via silica gel chromatography, eluting with 0-25% EtOAc in PE to afford the title compound (140 mg, crude) as a white solid. MS (ES+) C₂₇H₃₁FN₂O₄S requires: 499, found: 500 [M+H]⁺.

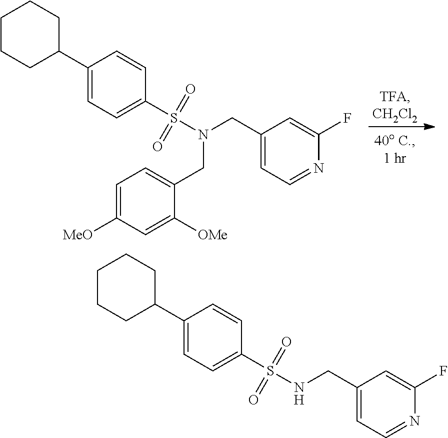

4-Cyclohexyl-N-((2-fluoropyridin-4-yl)methyl)benzenesulfonamide To a solution of the product from the previous step (70 mg, 0.140 mmol) in CH₂Cl₂ (2 mL) was add trifluoroacetic acid (138 mg, 1.403 mmol) dropwise at 0° C. The mixture was warmed to 40° C. and stirred for 1 hr. The solvent was evaporated under reduced pressure, and the residue was purified by prep-HPLC to afford the title compound (5.2 mg, 11%) as a white solid.

MS (ES+) C₁₈H₂₁FN₂O₂S requires: 348, found: 349 [M+H]⁺.

¹H NMR (400 MHz, DMSO-d₆) δ 8.09 (d, J=5.1 Hz, 1H), 7.67 (d, J=8.3 Hz, 2H), 7.39 (d, J=8.3 Hz, 2H), 7.20 (d, J=5.0 Hz, 1H), 6.94 (s, 1H), 6.78 (s, 1H), 4.12 (s, 2H), 2.57 (t, J=10.0 Hz, 1H), 1.83-1.67 (m, 5H), 1.46-1.21 (m, 5H).

### Example 9

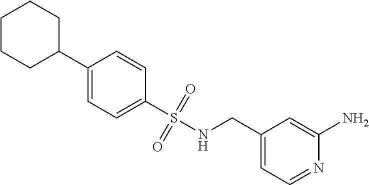 [SMILES: Nc1cc(CNS(=O)(=O)c2ccc(C3CCCCC3)cc2)ccn1]

### N-((2-aminopyridin-4-yl)methyl)-4-cyclohexylbenzenesulfonamide

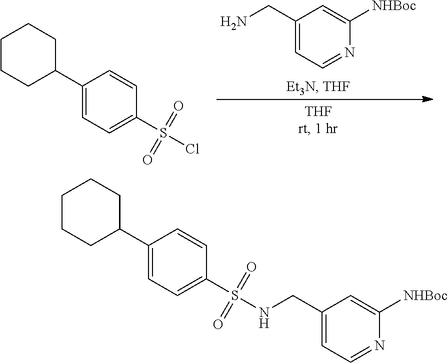 [SMILES: O=S(=O)(Cl)c1ccc(C2CCCCC2)cc1 ; CC(C)(C)OC(=O)Nc1cc(CN)ccn1 ; CC(C)(C)OC(=O)Nc1cc(CNS(=O)(=O)c2ccc(C3CCCCC3)cc2)ccn1]

tert-Butyl (4-(((4-cyclohexylphenyl)sulfonamido)methyl)pyridin-2-yl)carbamate A mixture of 4-cyclohexylbenzenesulfonyl chloride (206 mg, 0.8 mmol), tert-butyl (4-(aminomethyl)pyridin-2-yl)carbamate (150 mg, 0.67 mmol), and Et₃N (135 mg, 1.34 mmol) in THE (8 mL) was stirred at rt for 1 hr. The mixture was concentrated to give the crude product which was purified by silica gel chromatography (PE:EtOAc=1:2) to give the title compound (280 mg, 94%) as a yellow oil. MS (ES+) C₂₃H₃₁N₃O₄S requires: 445, found: 446 [M+H]⁺.

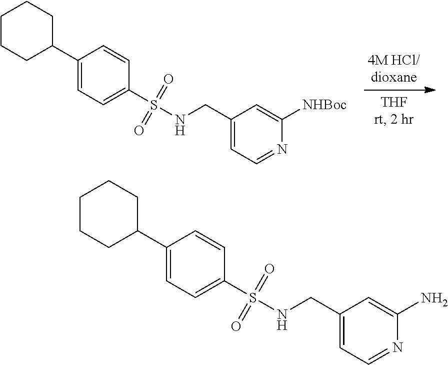 [SMILES: CC(C)(C)OC(=O)Nc1cc(CNS(=O)(=O)c2ccc(C3CCCCC3)cc2)ccn1 ; Nc1cc(CNS(=O)(=O)c2ccc(C3CCCCC3)cc2)ccn1]

N-((2-Aminopyridin-4-yl)methyl)-4-cyclohexylbenzenesulfonamide A mixture of the product from the previous step (280 mg, 0.63 mmol) in HCl/dioxane (4 M, 2 mL) and MeOH (2 mL) was stirred at rt for 2 hr. The mixture was concentrated and purified by preparative HPLC (NH₄HCO₃) to give the title compound (48 mg, 22%) as a white solid.

MS (ES+) C₁₈H₂₃N₃O₂S requires: 345, found: 346 [M+1]⁺.

¹H NMR (500 MHz, DMSO-d₆) δ 8.05 (s, 1H), 7.69-7.75 (m, 3H), 7.41-7.44 (m, 2H), 6.31-6.36 (m, 2H), 5.87 (s, 2H), 3.78 (s, 2H), 2.56-2.57 (m, 1H), 1.69-1.80 (m, 5H), 1.35-1.43 (m, 5H).

### Example 10

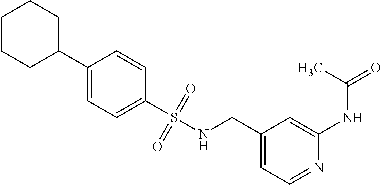 [SMILES: CC(=O)Nc1cc(CNS(=O)(=O)c2ccc(C3CCCCC3)cc2)ccn1]

### N-(4-(((4-cyclohexylphenyl)sulfonamido)methyl)pyridin-2-yl)acetamide

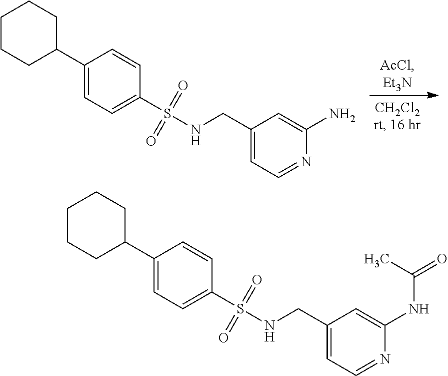 [SMILES: Nc1cc(CNS(=O)(=O)c2ccc(C3CCCCC3)cc2)ccn1 ; CC(=O)Nc1cc(CNS(=O)(=O)c2ccc(C3CCCCC3)cc2)ccn1]

To a solution of the Example 9 compound (60 mg, 0.17 mmol) and Et₃N (34 mg 0.34 mmol) in CH₂Cl₂ (10 mL) was added AcCl (27 mg, 0.34 mmol) at 0° C. The resulting mixture was stirred at 0° C. for 2 hr. The solvent was evaporated and the resulting solid was dissolved in MeOH (5 mL). K₂CO₃ (47 mg, 0.34 mmol) was added. The resulting mixture was stirred at rt for 16 hr. The solid was removed by filtration, and the filtrate was concentrated to give the crude product which was purified by preparative HPLC (NH₄HCO₃) to afford the title compound (40.8 mg, 62%) as a white solid.

MS (ES+) C₂₀H₂₅N₃O₃S requires: 386, found: 387 [M+1]⁺.

¹H NMR (500 MHz, DMSO-d₆) δ 10.44 (s, 1H), 8.13-8.35 (m, 3H), 7.67-7.69 (m, 2H), 7.38-7.47 (m, 2H), 6.92-6.93 (m, 1H), 3.97 (s, 2H), 2.55-2.60 (m, 1H), 2.07 (s, 3H), 1.69-1.79 (m, 5H), 1.23-1.43 (m, 5H).

### Example 11

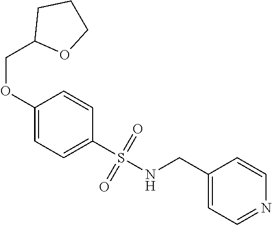 [SMILES: O=S(=O)(NCc1ccncc1)c1ccc(OCC2CCCO2)cc1]

### N-(pyridin-4-ylmethyl)-4-((tetrahydrofuran-2-yl)methoxy)benzenesulfonamide

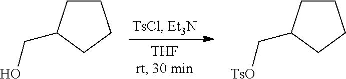 [SMILES: OCC1CCCC1 ; Cc1ccc(S(=O)(=O)OCC2CCCC2)cc1]

(Tetrahydrofuran-2-yl)methyl 4-methylbenzenesulfonate A mixture of (tetrahydrofuran-2-yl)methanol (510 mg, 5 mmol), TsCl (950 mg, 5 mmol) and Et₃N (1510 mg, 15 mmol) in THE (30 mL) was stirred at rt for 30 min. H₂O (20 ml) was added, and the mixture was extracted by EtOAc (20 ml). The combined organic layers were dried and concentrated to afford the title compound as a white solid (870 mg, 68%). MS (ES+) C₁₂H₁₆O₄S requires: 256, found: 257[M+H]⁺.

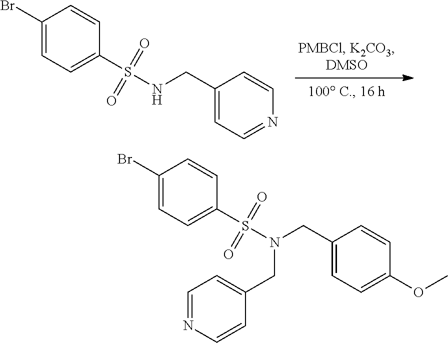

4-bromo-N-(4-methoxybenzyl)-N-(pyridin-4-ylmethyl)benzenesulfonamide A mixture of 4-bromo-N-(pyridin-4-ylmethyl)benzenesulfonamide (2 g, 6.1 mmol), 1-(chloromethyl)-4-methoxybenzene (1.4 g, 9.1 mmol), K₂CO₃ (2.5 g, 18 mmol) in DMSO (20 mL) was stirred at 100° C. for 16 h. The mixture was diluted with EtOAc, washed with brine, and the solvent was removed under reduced pressure. The residue was purified with silica gel chromatography to provide the title compound (2.6 g, 95%). MS (ES+) C20H19BrN2O3S requires: 446, 448, found: 447, 449 [M+H]⁺.

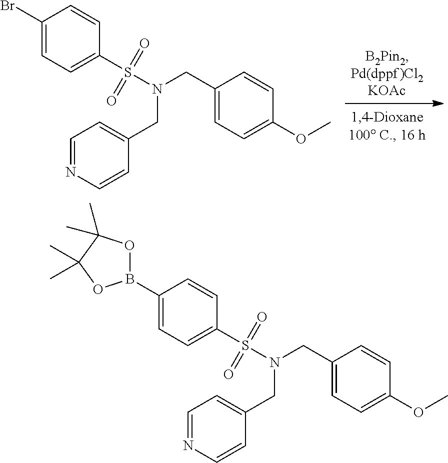

N-(4-methoxybenzyl)-N-(pyridin-4-ylmethyl)-4-(4,4,5,5-tetramethyl-1,3,2-dioxaborolan-2-yl)benzenesulfonamide A mixture of 4-bromo-N-(4-methoxybenzyl)-N-(pyridin-4-ylmethyl)benzenesulfonamide (2.6 g, 5.8 mmol), 4,4,4′,4′,5,5,5′,5′-octamethyl-2,2′-bi(1,3,2-dioxaborolane) (1.9 g, 7.5 mmol), KOAc (1.1 g, 12 mmol) and Pd(dppf)C₁₂ (223 mg, 0.3 mmol) in 1,4-Dioxane (25 mL) was stirred at 100° C. for 16 h. The mixture was diluted with EtOAc and washed with brine. The solvent was removed under reduced pressure to provide the title compound that was used in next step without further purification, (2.4 g, 84%). MS (ES+) C26H31BN2O5S requires: 494, found: 495 [M+H]⁺.

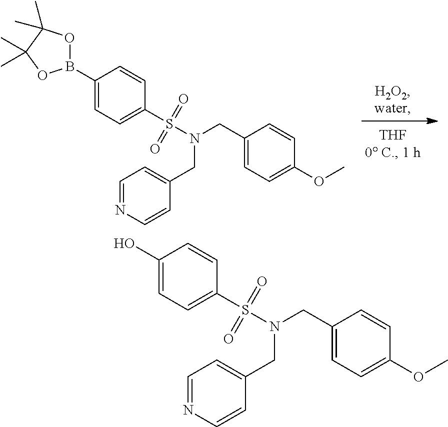

4-hydroxy-N-(4-methoxybenzyl)-N-(pyridin-4-ylmethyl)benzenesulfonamide To a mixture of N-(4-methoxybenzyl)-N-(pyridin-4-ylmethyl)-4-(4,4,5,5-tetramethyl-1,3,2-dioxaborolan-2-yl)benzenesulfonamide (2.4 g, 4.8 mmol) in water (20 ml) and THF (20 ml), H₂O₂ (30%, 5 eq) was added dropwise at 0° C. The mixture was stirred for 1 h, diluted with water, and extracted with EtOAc (3×20 mL), washed with aq. Na₂S₂O₃ and brine, dried and purified with silica gel chromatography to provide the title compound (750 mg, 40%). MS (ES+) C₂₀H₂ON2O4S requires: 384, found: 385[M+H]⁺.

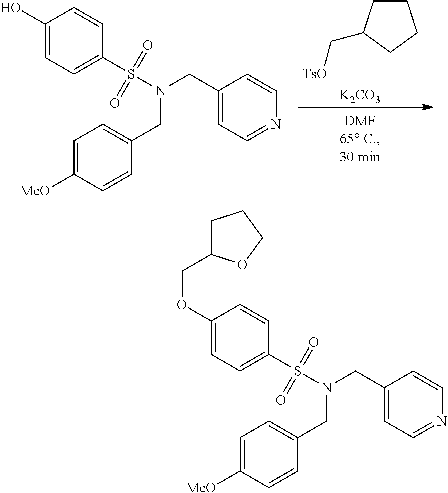

N-(4-Methoxybenzyl)-N-(pyridin-4-ylmethyl)-4-((tetrahydrofuran-2-yl)methoxy)benzenesulfonamide A mixture of the product from the previous step (33 mg, 0.13 mmol), 4-hydroxy-N-(4-methoxybenzyl)-N-(pyridin-4-ylmethyl)benzenesulfonamide (50 mg, 0.13 mmol), and K₂CO₃ (54 mg, 0.39 mmol) in DMF (3 mL) was stirred at 65° C. for 30 min. H₂O (20 ml) was added, and the mixture was extracted with EtOAc (20 ml). The combined organic phases were dried and the residue was concentrated to afford the title compound as white solid (55 mg, 91%). MS (ES+) C₂₅H₂₈N₂O₅S requires: 468, found: 469[M+H]⁺.

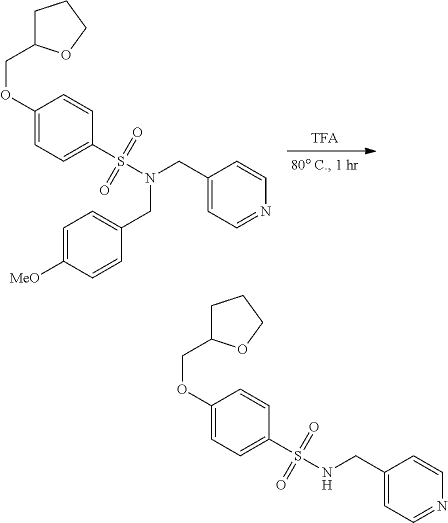 [SMILES: COc1ccc(CN(Cc2ccncc2)S(=O)(=O)c2ccc(OCC3CCCO3)cc2)cc1 ; O=S(=O)(NCc1ccncc1)c1ccc(OCC2CCCO2)cc1]

N-(Pyridin-4-ylmethyl)-4-((tetrahydrofuran-2-yl)methoxy)benzenesulfonamide A mixture of the product from the previous step (55 mg, 0.11 mmol) in 3 ml TFA was stirred at 80° C. for 1 hr. The mixture was concentrated and purified by prep-HPLC (0.1% TFA in H₂O:CH₃CN=10-50%, C18) to afford the title compound (7 mg, 18%) as a white solid.

MS (ES+) C₁₇H₂₀N₂O₄S requires: 348, found: 349[M+H]⁺.

¹H NMR (400 MHz, CD₃OD) δ 8.43-8.42 (m, 2H), 7.79-7.77 (m, 2H), 7.35-7.34 (m, 2H), 7.09-7.07 (m, 2H), 4.31-4.28 (m, 1H), 4.13-3.83 (m, 6H), 2.01-1.81 (m, 4H).

The following Examples were synthesized with procedures that were similar to the examples disclosed herein, and can generally be made by methods disclosed herein. The Examples may be made as free bases or as TFA salts.

TABLE 1
PU.1 Inhibitors.

| Ex. | | | Proc. |
|---|---|---|---|
| No. | Structure | IUPAC name | Ex. No. |
| 12 | 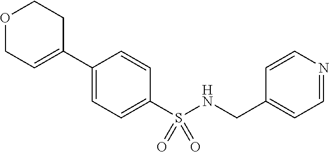 [SMILES: O=S(=O)(NCc1ccncc1)c1ccc(C2=CCOCC2)cc1] | 4-(3,6-dihydro-2H-pyran-4- yl)-N-(pyridin-4-ylmethyl)- benzenesulfonamide | 1 |
| 13 | 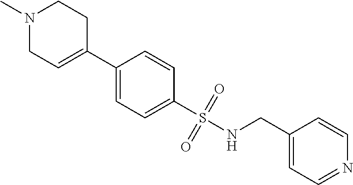 [SMILES: CN1CC=C(c2ccc(S(=O)(=O)NCc3ccncc3)cc2)CC1] | 4-(1-methyl-1,2,3,6- tetrahydropyridin-4-yl)-N- (pyridin-4-ylmethyl)- benzenesulfonamide | 1 |
| 14 | 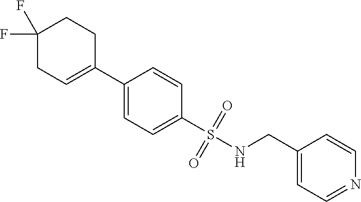 [SMILES: O=S(=O)(NCc1ccncc1)c1ccc(C2=CCC(F)(F)CC2)cc1] | 4′,4′-difluoro-N-(pyridin-4- ylmethyl)-2′,3′,4′,5′- tetrahydro-[1,1′-biphenyl]- 4-sulfonamide | 1 |
| 15 | 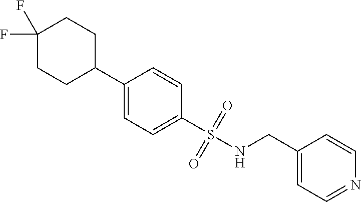 [SMILES: O=S(=O)(NCc1ccncc1)c1ccc(C2CCC(F)(F)CC2)cc1] | 4-(4,4-difluorocyclohexyl)- N-(pyridin-4-ylmethyl)- benzenesulfonamide | 2 |
| 16 | 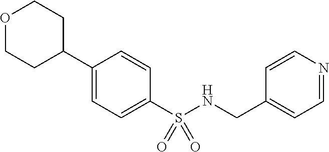 [SMILES: O=S(=O)(NCc1ccncc1)c1ccc(C2CCOCC2)cc1] | N-(pyridin-4-ylmethyl)-4- (tetrahydro-2H-pyran-4-yl)- benzenesulfonamide | 2 |
| 17 | 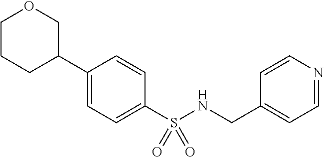 [SMILES: O=S(=O)(NCc1ccncc1)c1ccc(C2CCCOC2)cc1] | N-(pyridin-4-ylmethyl)-4- (tetrahydro-2H-pyran-3-yl)- benzenesulfonamide | 2 |
| 18 | 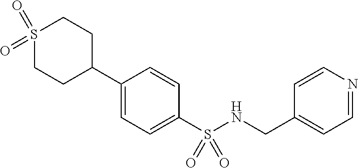 [SMILES: O=S1(=O)CCC(c2ccc(S(=O)(=O)NCc3ccncc3)cc2)CC1] | 4-(1,1-dioxotetrahydro- 2H-thiopyran-4-yl)-N- (pyridin-4-ylmethyl)- benzenesulfonamide | 2 |
| 19 | 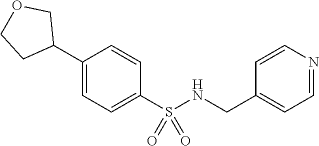 [SMILES: O=S(=O)(NCc1ccncc1)c1ccc(C2CCOC2)cc1] | N-(pyridin-4-ylmethyl)-4- (tetrahydrofuran-3-yl)- benzenesulfonamide | 2 |
| 20 | 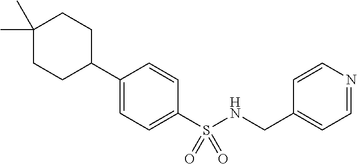 [SMILES: CC1(C)CCC(c2ccc(S(=O)(=O)NCc3ccncc3)cc2)CC1] | 4-(4,4-dimethyl- cyclohexyl)-N-(pyridin-4- ylmethyl) benzenesulfonamide | 2 |
| 21 | 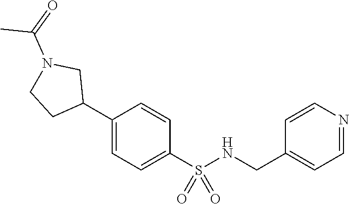 [SMILES: CC(=O)N1CCC(c2ccc(S(=O)(=O)NCc3ccncc3)cc2)C1] | 4-(1-acetylpyrrolidin-3-yl)- N-(pyridin-4-ylmethyl)- benzenesulfonamide | 2 |
| 22 | 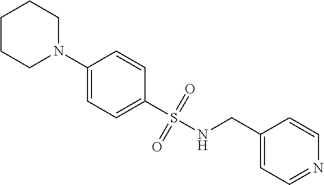 [SMILES: O=S(=O)(NCc1ccncc1)c1ccc(N2CCCCC2)cc1] | 4-(piperidin-1-yl)-N- (pyridin-4-ylmethyl)- benzenesulfonamide | 3 |
| 23 | 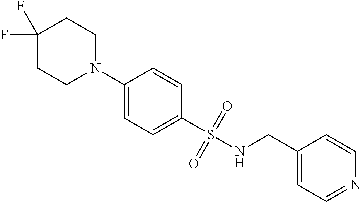 [SMILES: O=S(=O)(NCc1ccncc1)c1ccc(N2CCC(F)(F)CC2)cc1] | 4-(4,4-difluoropiperidin-1- yl)-N-(pyridin-4-ylmethyl)- benzenesulfonamide | 3 |
| 24 | 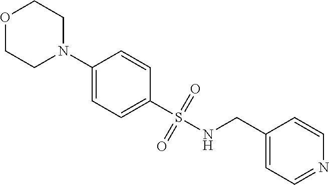 [SMILES: O=S(=O)(NCc1ccncc1)c1ccc(N2CCOCC2)cc1] | 4-morpholino-N-(pyridin-4- ylmethyl)benzenesulfonamide | 3 |
| 25 | 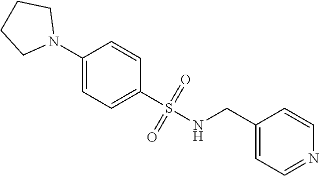 [SMILES: O=S(=O)(NCc1ccncc1)c1ccc(N2CCCC2)cc1] | N-(pyridin-4-ylmethyl)-4- (pyrrolidin-1-yl)benzenesulfonamide | 3 |
| 26 | 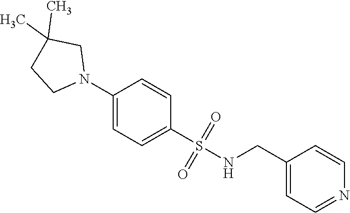 [SMILES: CC1(C)CCN(c2ccc(S(=O)(=O)NCc3ccncc3)cc2)C1] | 4-(3,3-dimethylpyrrolidin- 1-yl)-N-(pyridin-4-yl- methyl)benzenesulfonamide | 3 |
| 27 | 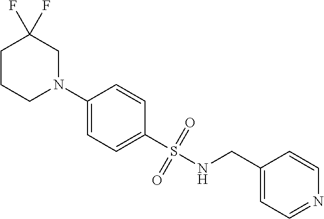 [SMILES: O=S(=O)(NCc1ccncc1)c1ccc(N2CCCC(F)(F)C2)cc1] | 4-(3,3-difluoropiperidin-1- yl)-N-(pyridin-4-ylmethyl)- benzenesulfonamide | 3 |
| 28 | 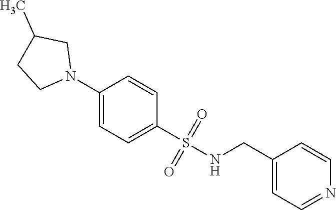 [SMILES: CC1CCN(c2ccc(S(=O)(=O)NCc3ccncc3)cc2)C1] | 4-(3-methylpyrrolidin-1- yl)-N-(pyridin-4-ylmethyl)- benzenesulfonamide | 3 |
| 29 | 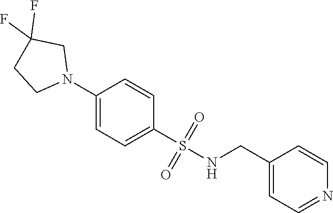 [SMILES: O=S(=O)(NCc1ccncc1)c1ccc(N2CCC(F)(F)C2)cc1] | 4-(3,3-difluoropyrrolidin-1- yl)-N-(pyridin-4-ylmethyl)- benzenesulfonamide | 3 |
| 30 | 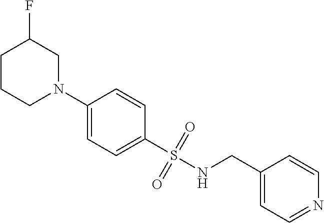 [SMILES: O=S(=O)(NCc1ccncc1)c1ccc(N2CCCC(F)C2)cc1] | 4-(3-fluoropiperidin-1-yl)- N-(pyridin-4-ylmethyl)- benzenesulfonamide | 3 |
| 31 | 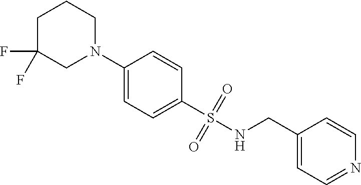 [SMILES: O=S(=O)(NCc1ccncc1)c1ccc(N2CCCC(F)(F)C2)cc1] | 4-(3,3-difluoropiperidin-1- yl)-N-(pyridin-4-ylmethyl)- benzenesulfonamide | 3 |
| 32 | 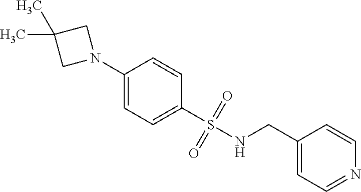 [SMILES: CC1(C)CN(c2ccc(S(=O)(=O)NCc3ccncc3)cc2)C1] | 4-(3,3-dimethylazetidin-1- yl)-N-[(pyridin-4- yl)methyl]benzene-1- sulfonamide | 3 |
| 33 | 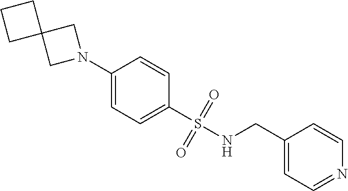 [SMILES: O=S(=O)(NCc1ccncc1)c1ccc(N2CC3(CCC3)C2)cc1] | N-(pyridin-4-ylmethyl)-4- (2-azaspiro[3.3]heptan-2- yl)benzenesulfonamide | 3 |
| 34 | 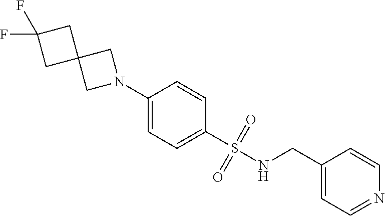 [SMILES: O=S(=O)(NCc1ccncc1)c1ccc(N2CC3(C2)CC(F)(F)C3)cc1] | 4-(6,6-difluoro-2- azaspiro[3.3]heptan-2-yl)- N-(pyridin-4-ylmethyl)- benzenesulfonamide | 3 |
| 35 | 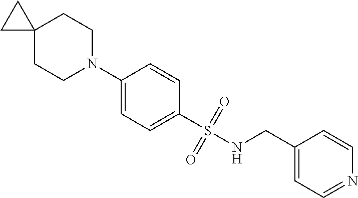 [SMILES: O=S(=O)(NCc1ccncc1)c1ccc(N2CCC3(CC2)CC3)cc1] | N-(pyridin-4-ylmethyl)-4- (6-azaspiro[2.5]octan-6-yl)- benzenesulfonamide | 3 |
| 36 | 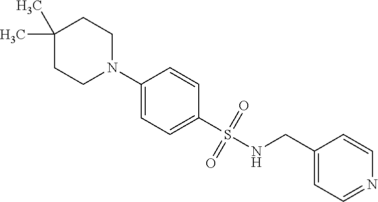 [SMILES: CC1(C)CCN(c2ccc(S(=O)(=O)NCc3ccncc3)cc2)CC1] | 4-(4,4-dimethylpiperidin-1- yl)-N-(pyridin-4-ylmethyl)- benzenesulfonamide | 3 |
| 37 | 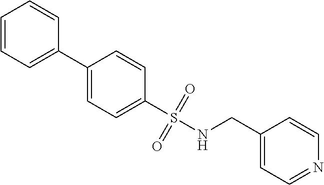 [SMILES: O=S(=O)(NCc1ccncc1)c1ccc(-c2ccccc2)cc1] | N-(pyridin-4-ylmethyl)- [1,1′-biphenyl]-4- sulfonamide | 4 |
| 38 | 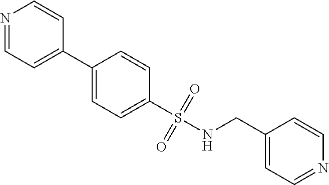 [SMILES: O=S(=O)(NCc1ccncc1)c1ccc(-c2ccncc2)cc1] | 4-(pyridin-4-yl)-N-(pyridin- 4-ylmethyl)benzenesulfonamide | 4 |
| 39 | 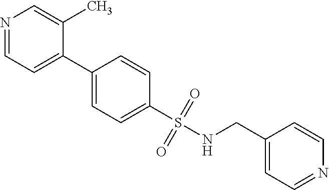 [SMILES: Cc1cnccc1-c1ccc(S(=O)(=O)NCc2ccncc2)cc1] | 4-(3-methylpyridin-4-yl)- N-(pyridin-4-ylmethyl)- benzenesulfonamide | 4 |
| 40 | 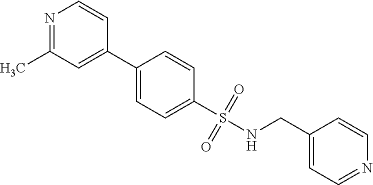 [SMILES: Cc1cc(-c2ccc(S(=O)(=O)NCc3ccncc3)cc2)ccn1] | 4-(2-methylpyridin-4-yl)- N-(pyridin-4-ylmethyl)- benzenesulfonamide | 4 |
| 41 | 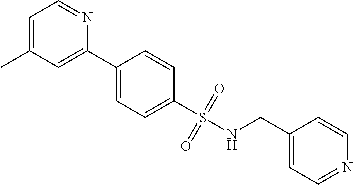 [SMILES: Cc1ccnc(-c2ccc(S(=O)(=O)NCc3ccncc3)cc2)c1] | 4-(4-methylpyridin-2-yl)- N-(pyridin-4-ylmethyl)- benzenesulfonamide | 5 |
| 42 | 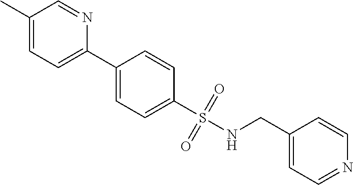 [SMILES: Cc1ccc(-c2ccc(S(=O)(=O)NCc3ccncc3)cc2)nc1] | 4-(5-methylpyridin-2-yl)- N-(pyridin-4-ylmethyl)- benzenesulfonamide | 5 |
| 43 | 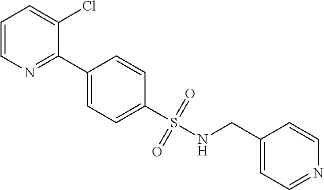 [SMILES: O=S(=O)(NCc1ccncc1)c1ccc(-c2ncccc2Cl)cc1] | 4-(3-chloropyridin-2-yl)-N- (pyridin-4-ylmethyl)- benzenesulfonamide | 5 |
| 44 | 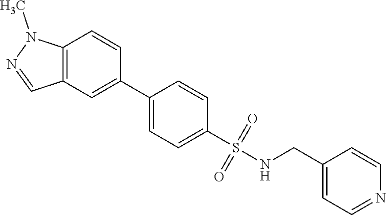 | 4-(1-methyl-1H-indazol-5- yl)-N-(pyridin-4-ylmethyl)- benzenesulfonamide | 5 |
| 45 | 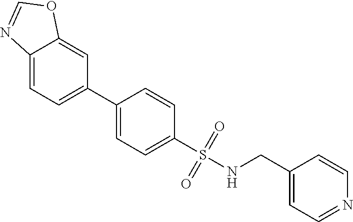 [SMILES: O=S(=O)(NCc1ccncc1)c1ccc(-c2ccc3ncoc3c2)cc1] | 4-(benzo[d]oxazol-6-yl)-N- (pyridin-4-ylmethyl)- benzenesulfonamide | 5 |
| 46 | 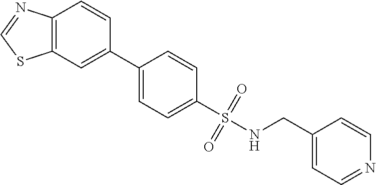 [SMILES: O=S(=O)(NCc1ccncc1)c1ccc(-c2ccc3ncsc3c2)cc1] | 4-(benzo[d]thiazol-6-yl)-N- (pyridin-4-ylmethyl)- benzenesulfonamide | 5 |
| 47 | 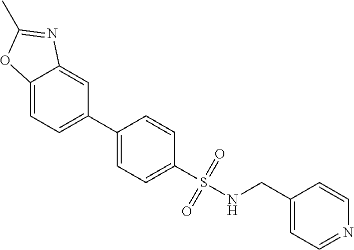 [SMILES: Cc1nc2cc(-c3ccc(S(=O)(=O)NCc4ccncc4)cc3)ccc2o1] | 4-(2-methyl- benzo[d]oxazol-5-yl)-N- (pyridin-4-ylmethyl)- benzenesulfonamide | 5 |
| 48 | 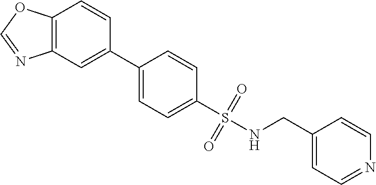 [SMILES: O=S(=O)(NCc1ccncc1)c1ccc(-c2ccc3ocnc3c2)cc1] | 4-(1,3-benzoxazol-5-yl)-N- [(pyridin-4- yl)methyl]benzene-1- sulfonamide | 5 |
| 49 | 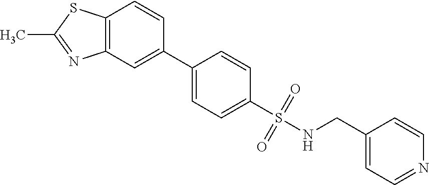 [SMILES: Cc1nc2cc(-c3ccc(S(=O)(=O)NCc4ccncc4)cc3)ccc2s1] | 4-(2-methyl- benzo[d]thiazol-5-yl)-N- (pyridin-4-ylmethyl)- benzenesulfonamide | 5 |
| 50 | 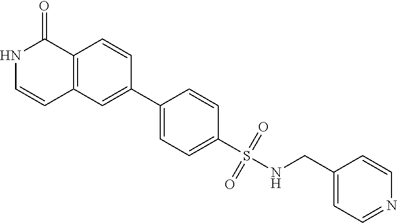 [SMILES: O=c1[nH]ccc2cc(-c3ccc(S(=O)(=O)NCc4ccncc4)cc3)ccc12] | 4-(1-oxo-1,2-dihydro- isoquinolin-6-yl)-N- (pyridin-4-ylmethyl)- benzenesulfonamide | 5 |
| 51 | 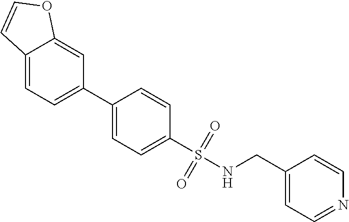 [SMILES: O=S(=O)(NCc1ccncc1)c1ccc(-c2ccc3ccoc3c2)cc1] | 4-(benzofuran-6-yl)-N- (pyridin-4-ylmethyl)- benzenesulfonamide | 5 |
| 52 | 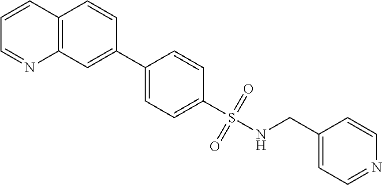 [SMILES: O=S(=O)(NCc1ccncc1)c1ccc(-c2ccc3cccnc3c2)cc1] | N-(pyridin-4-ylmethyl)-4- (quinolin-7-yl)benzenesulfonamide | 5 |
| 53 | 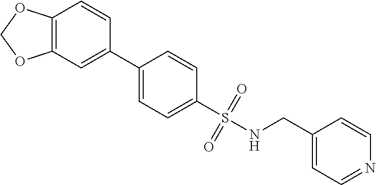 [SMILES: O=S(=O)(NCc1ccncc1)c1ccc(-c2ccc3c(c2)OCO3)cc1] | 4-(benzo[d][1,3]dioxol-5- yl)-N-(pyridin-4-ylmethyl)- benzenesulfonamide | 5 |
| 54 | 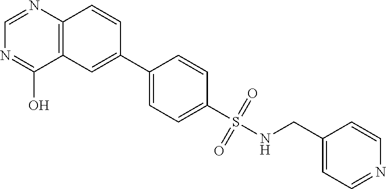 [SMILES: O=S(=O)(NCc1ccncc1)c1ccc(-c2ccc3ncnc(O)c3c2)cc1] | 4-(4-hydroxyquinazolin-6- yl)-N-(pyridin-4-ylmethyl)- benzenesulfonamide | 5 |
| 55 | 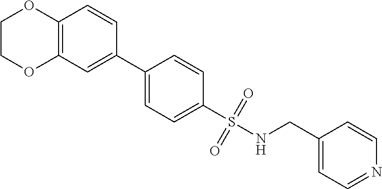 [SMILES: O=S(=O)(NCc1ccncc1)c1ccc(-c2ccc3c(c2)OCCO3)cc1] | 4-(2,3-dihydrobenzo[b]- [1,4]dioxin-6-yl)-N- (pyridin-4-ylmethyl)- benzenesulfonamide | 5 |
| 56 | 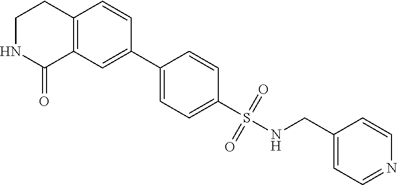 [SMILES: O=C1NCCc2ccc(-c3ccc(S(=O)(=O)NCc4ccncc4)cc3)cc21] | 4-(1-oxo-1,2,3,4- tetrahydroisoquinolin-7-yl)- N-(pyridin-4-ylmethyl)- benzenesulfonamide | 5 |
| 57 | 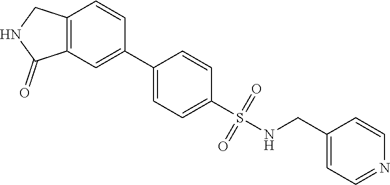 [SMILES: O=C1NCc2ccc(-c3ccc(S(=O)(=O)NCc4ccncc4)cc3)cc21] | 4-(3-oxoisoindolin-5-yl)-N- (pyridin-4-ylmethyl)- benzenesulfonamide | 5 |
| 58 | 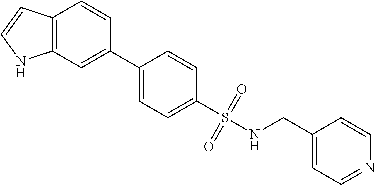 [SMILES: O=S(=O)(NCc1ccncc1)c1ccc(-c2ccc3cc[nH]c3c2)cc1] | 4-(1H-indol-6-yl)-N- (pyridin-4-ylmethyl)- benzenesulfonamide | 5 |
| 59 | 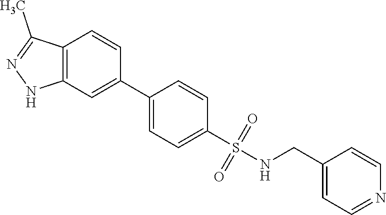 | 4-(3-methyl-1H-indazol-6- yl)-N-(pyridin-4-ylmethyl)- benzenesulfonamide | 5 |
| 60 | 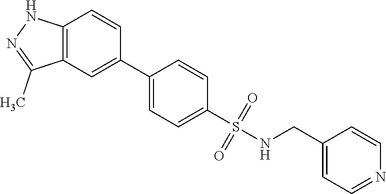 [SMILES: Cc1n[nH]c2ccc(-c3ccc(S(=O)(=O)NCc4ccncc4)cc3)cc12] | 4-(3-methyl-1H-indazol-6- yl)-N-[(pyridin-4- yl)methyl]benzene-1- sulfonamide | 5 |
| 61 | 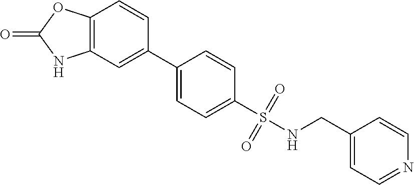 [SMILES: O=c1[nH]c2cc(-c3ccc(S(=O)(=O)NCc4ccncc4)cc3)ccc2o1] | 4-(2-oxo-2,3-dihydro- benzo[d]oxazol-5-yl)-N- (pyridin-4-ylmethyl)- benzenesulfonamide | 5 |
| 62 | 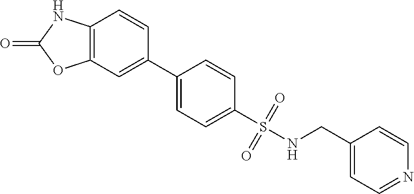 [SMILES: O=c1[nH]c2ccc(-c3ccc(S(=O)(=O)NCc4ccncc4)cc3)cc2o1] | 4-(2-oxo-2,3-dihydro- benzo[d]oxazol-6-yl)-N- (pyridin-4-ylmethyl)- benzenesulfonamide | 5 |
| 63 | 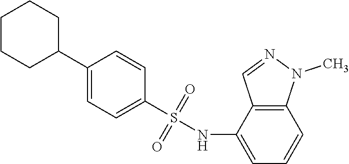 [SMILES: Cn1ncc2c(NS(=O)(=O)c3ccc(C4CCCCC4)cc3)cccc21] | 4-cyclohexyl-N-(1-methyl- 1H-indazol-4-yl)benzenesulfonamide | 6 |

TABLE 2
Analytical data for PU.1 inhibitors.

| | Calc. Mass/ | |
|---|---|---|
| Ex. | Obsd. Mass | |
| No. | [M + H]⁺ | ¹H NMR |
| 12 | 330/331 | (a) δ 8.42-8.39 (m, 2H), 7.81-7.80 (m, 2H), 7.61-7.60 (m, 2H), 7.34- |
| | | 7.33 (m, 2H), 6.38 (m, 1H), 4.35-4.33 (m, 2H), 4.17 (s, 2H), 3.96-3.94 |
| | | (m, 2H), 2.56-2.55 (m, 2H) |
| 13 | 343/344 | (a) δ 8.41-8.39 (m, 2H), 7.79-7.76 (m, 2H), 7.46-7.41 (m, 2H), 7.31- |
| | | 7.30 (m, 2H), 6.31 (m, 1H), 4.16-4.06 (m, 4H), 3.53-3.50 (m, 2H), 3.48- |
| | | 3.46 (m, 2H), 2.67-2.63 (m, 1H), 2.36 (s, 3H), 1.86-1.76 (m, 4H) |
| 14 | 364/365 | (a) δ 8.42-8.39 (m, 2H), 7.81-7.80 (m, 2H), 7.61-7.60 (m, 2H), 7.34- |
| | | 7.33 (m, 2H), 6.13 (m, 1H), 4.17 (s, 2H), 2.78-2.73 (m, 4H), 2.25-2.19 |
| | | (m, 2H) |
| 15 | 366/367 | (a) δ 8.42-8.39 (m, 2H), 7.81-7.80 (m, 2H), 7.61-7.60 (m, 2H), 7.34- |
| | | 7.33 (m, 2H), 4.17 (s, 2H), 2.82-2.77 (m, 1H), 2.20-1.79 (m, 8H). |
| 16 | 332/333 | (a) δ 8.41-8.39 (m, 2H), 7.79-7.76 (m, 2H), 7.46-7.31 (m, 4H), 4.16- |
| | | 4.06 (m, 4H), 3.53-3.50 (m, 2H), 3.48-3.46 (m, 2H), 2.94 (m, 1H), 2.03- |
| | | 1.76 (m, 4H) |
| 17 | 332/333 | (a) δ 8.41-8.39 (m, 2H), 7.79-7.76 (m, 2H), 7.46-7.31 (m, 4H), 4.16- |
| | | 4.06 (m, 4H), 3.53-3.50 (m, 2H), 3.48-3.46 (m, 2H), 2.94 (m, 1H), 2.03- |
| | | 1.76 (m, 4H) |
| 18 | 387/388 | (a) δ 8.42-8.40 (m, 2H), 7.66-7.63 (m, 2H), 7.34-7.32 (m, 2H), 7.02- |
| | | 6.98 (m, 2H), 4.11 (s, 2H), 3.37-3.33 (m, 5H), 2.34-2.19 (m, 4H) |
| 19 | 318/319 | (a) δ 8.41-8.39 (m, 2H), 7.79-7.76 (m, 2H), 7.46-7.31 (m, 4H), 4.16- |
| | | 4.06 (m, 4H), 3.95-3.89 (m, 1H), 3.75-3.72 (m, 1H), 3.55-3.51 (m, 1H), |
| | | 2.45-2.43 (m, 1H), 2.05-2.00 (m, 1H) |
| 20 | 358/359 | (a) δ 8.40-8.39 (m, 2H), 7.76-7.74 (m, 2H), 7.42-7.31 (m, 4H), 4.15 (s, |
| | | 2H), 2.52 (m, 1H), 1.72-1.41 (m, 8H), 1.04-0.99 (m, 6H) |
| 21 | 359/360 | (a) δ 8.41 (s, 2H), 7.81 (d, J = 8.1 Hz, 2H), 7.49 (t, J = 8.2 Hz, 2H), 7.33 |
| | | (d, J = 4.4 Hz, 2H), 4.17 (d, J = 2.6 Hz, 2H), 4.04-3.94 (m, 1H), 3.77 |
| | | (dd, J = 20.2, 9.5 Hz, 1H), 3.68-3.57 (m, 1H), 3.48 (ddd, J = 42.9, |
| | | 25.8, 10.2 Hz, 2H), 2.48-2.31 (m, 1H), 2.19-2.03 (m, 4H), J = 25.5, |
| | | 12.8 Hz, 5H) |
| 22 | 331/332 | (a) δ 8.42-8.40 (m, 2H), 7.66-7.63 (m, 2H), 7.34-7.32 (m, 2H), 7.02- |
| | | 6.98 (m, 2H), 4.11 (s, 2H), 3.37-3.33 (m, 4H), 1.69 (m, 6H) |
| 23 | 367/368 | (a) δ 8.42-8.40 (m, 2H), 7.66-7.63 (m, 2H), 7.34-7.32 (m, 2H), 7.02- |
| | | 6.98 (m, 2H), 4.11 (s, 2H), 3.57-3.55 (m, 4H), 2.08-2.05 (m, 4H) |
| 24 | 333/334 | (a) δ 8.42-8.40 (m, 2H), 7.66-7.63 (m, 2H), 7.34-7.32 (m, 2H), 7.02- |
| | | 6.98 (m, 2H), 4.11 (s, 2H), 3.85-3.83 (m, 4H), 3.33-3.29 (m, 4H) |
| 25 | 317/318 | (a) δ 8.42-8.40 (m, 2H), 7.66-7.63 (m, 2H), 7.34-7.32 (m, 2H), 7.02- |
| | | 6.98 (m,2H), 4.11 (s, 2H), 3.37-3.33 (m, 4H), 2.09-2.06 (m, 4H) |
| 26 | 345/346 | (a) δ 8.42-8.40 (m, 2H), 7.66-7.63 (m, 2H), 7.34-7.32 (m, 2H), 7.02- |
| | | 6.98 (m, 2H),4.11 (s, 2H), 3.46-3.43 (m, 2H),3.13 (s, 2H), 1.88-1.85 (m, |
| | | 2H), 1.18 (s, 6H) |
| 27 | 367/368 | (a) δ 8.42-8.40 (m, 2H), 7.66-7.63 (m, 2H), 7.34-7.32 (m, 2H), 7.02- |
| | | 6.98 (m, 2H), 4.11 (s, 2H), 3.67-3.43 (m, 4H), 2.14-2.05 (m, 2H), 1.91- |
| | | 1.87 (m, 2H) |
| 28 | 331/332 | (a) δ 8.42-8.40 (m, 2H), 7.66-7.63 (m, 2H), 7.34-7.32 (m, 2H), 7.02- |
| | | 6.98 (m, 2H), 4.11(s, 2H), 3.53-3.43 (m, 3H), 2.94-2.90 (m, 1H), 2.90- |
| | | 2.88 |
| 29 | 353/354 | (a) δ 8.42-8.40 (m, 2H), 7.66-7.63 (m, 2H), 7.34-7.32 (m, 2H), 7.02- |
| | | 6.98 (m, 2H), 4.11 (s, 2H), 3.79-3.60 (m, 4H), 2.57-2.54 (m, 2H) |
| 30 | 349/350 | (a) δ 8.42-8.40 (m, 2H), 7.66-7.63 (m, 2H), 7.34-7.32 (m, 2H), 7.02- |
| | | 6.98 (m, 2H), 4.83-4.69 (m, 1H), 4.11 (s, 2H), 3.59-3.44 (m, 4H), 2.05- |
| | | 1.88 (m, 4H) |
| 31 | 359/360 | (a) δ 8.42-8.41 (m, 2H), 7.62-7.60 (m, 2H), 7.34-7.33 (m, 2H), 6.95- |
| | | 6.93 (m, 2H), 4.10 (s, 2H), 3.31-3.32 (m, 2H), 3.09 (m, 2H), 1.73 (m, |
| | | 2H), 1.49-1.46 (m, 2H), 1.01 (m, 6H) |
| 32 | 331/332 | (a) δ 8.46-8.45 (m, 2H), 8.00 (m, 1H), 7.63-7.61 (m, 2H), 7.25-7.24 (m, |
| | | 2H), 6.64-6.63 (m, 2H), 3.95 (s, 2H) 3.63.-3.61 (m, 4H), 1.29 (s, 6H) |
| 33 | 343/344 | (a) δ 8.42-8.40 (m, 2H), 7.66-7.63 (m, 2H), 7.34-7.32 (m, 2H), 7.02- |
| | | 6.98 (m, 2H), 4.08 (s, 2H), 3.91 (s, 4H), 2.29-2.24 (m, 4H), 1.94-1.90 |
| | | (m, 2H) |
| 34 | 379/380 | (a) δ 8.42-8.40 (m, 2H), 7.64-7.61 (m, 2H), 7.33-7.32 (m, 2H), 6.50- |
| | | 6.48 (m, 2H), 4.08-4.04 (m, 6H), 2.89-2.83 (m, 4H) |
| 35 | 357/358 | (a) δ 8.42-8.41 (m, 2H), 7.65-7.63 (m, 2H), 7.35-7.33 (m, 2H), 7.01- |
| | | 6.99 (m, 2H), 4.11 (s, 2H), 3.48-3.45 (m, 4H), 1.52-1.50 (m, 4H), 0.41 |
| | | (m, 4H) |
| 36 | 359/360 | (a) δ 8.42-8.41 (m, 2H), 7.64-7.63 (m, 2H), 7.35-7.34 (m, 2H), 6.99- |
| | | 6.97 (m, 2H), 4.11 (s, 2H), 3.39-3.37 (m, 4H), 1.52-1.50 (m, 4H), 1.04 |
| | | (m, 6H) |
| 37 | 324/325 | (a) δ 8.42-8.41 (m, 2H), 7.92-7.68 (m, 6H), 7.52-7.35 (m, 5H), 4.20 (s, |
| | | 2H) |
| 38 | 325/326 | (a) δ 8.70-8.69 (m, 2H), 8.45-8.43 (m, 2H), 7.98-7.75 (m, 6H), 7.27- |
| | | 7.26 (m, 2H), 4.06 (s, 2H) |
| 39 | 339/340 | (a) δ 8.52-8.42 (m, 4H), 7.96-7.95 (m, 2H), 7.57-7.55 (m, 2H), 7.36- |
| | | 7.31 (m, 3H), 4.26 (s, 2H), 2.31 (s, 3H) |
| 40 | 339/340 | (a) δ 8.53-8.52 (m, 1H), 8.42-8.41 (m, 2H), 7.98-7.89 (m, 4H), 7.65- |
| | | 7.55 (m, 2H), 7.36-7.34 (m, 2H), 4.22 (s, 2H), 2.64 (s, 3H) |
| 41 | 339/340 | (d) δ 8.57 (d, J = 5.0 Hz, 1H), 8.45 (d, J = 6.0 Hz, 2H), 8.39 (t, J = 6.4 |
| | | Hz, 1H), 8.26 (d, J = 8.6 Hz, 2H), 7.95-7.86 (m, 3H), 7.27 (d, J = 5.8 |
| | | Hz, 3H), 4.09 (d, J = 6.3 Hz, 2H), 2.42 (s, 3H) |
| 42 | 339/340 | (d) δ 8.56 (s, 1H), 8.45 (dd, J = 4.4, 1.6 Hz, 2H), 8.38 (t, J = 6.4 Hz, |
| | | 1H), 8.25 (d, J = 8.6 Hz, 2H), 7.97 (d, J = 8.2 Hz, 1H), 7.88 (d, J = 8.6 |
| | | Hz, 2H), 7.77 (dd, J = 8.1, 1.6 Hz, 1H), 7.28 (d, J = 6.0 Hz, 2H), 4.08 |
| | | (d, J = 6.4 Hz, 2H), 2.37 (s, 3H) |
| 43 | 360/361 | (d) δ 8.68 (dd, J = 4.6, 1.4 Hz, 1H), 8.46 (dd, J = 4.4, 1.6 Hz, 2H), 8.11 |
| | | (dd, J = 8.2, 1.4 Hz, 1H), 7.89 (td, J = 8.6, 6.5 Hz, 4H), 7.52 (dd, J = |
| | | 8.2, 4.6 Hz, 1H), 7.28 (d, J = 6.0 Hz, 2H), 4.12 (s, 2H) |
| 44 | 378/379 | (d) δ 8.46 (d, J = 5.9 Hz, 2H), 8.35 (t, J = 6.3 Hz, 1H), 8.14 (d, J = 6.5 |
| | | Hz, 2H), 7.89 (dd, J = 21.4, 8.6 Hz, 4H), 7.78 (s, 2H), 7.28 (d, J = 5.9 |
| | | Hz, 2H), 4.08 (d, J = 6.2 Hz, 5H) |
| 45 | 365/366 | (d) δ 8.84 (s, 1H), 8.46 (d, J = 5.9 Hz, 2H), 8.40 (s, 1H), 8.19 (d, J = 1.2 |
| | | Hz, 1H), 7.98-7.85 (m, 5H), 7.78 (dd, J = 8.4, 1.7 Hz, 1H), 7.28 (d, |
| | | J = 5.9 Hz, 2H), 4.09 (d, J = 5.5 Hz, 2H) |
| 46 | 381/382 | (b) δ 9.33 (s, 1H), 8.46-8.40 (m, 3H), 8.20 (d, J = 8.7 Hz, 1H), 7.97- |
| | | 7.88 (m, 5H), 7.37 (d, J = 6.1 Hz, 2H), 4.23 (s, 2H) |
| 47 | 379/380 | (b) δ 8.42 (d, J = 6.1 Hz, 2H), 7.93 (t, J = 6.1 Hz, 3H), 7.84 (d, J = 8.6 |
| | | Hz, 2H), 7.70 (d, J = 0.9 Hz, 2H), 7.38 (s, 2H), 4.22 (s, 2H), 2.70 (s, |
| | | 3H) |
| 48 | 365/366 | (d) δ 8.84 (s, 1H), 8.46 (dd, J = 4.5, 1.5 Hz, 2H), 8.17 (d, J = 1.5 Hz, |
| | | 1H), 7.99-7.77 (m, 7H), 7.28 (d, J = 5.9 Hz, 2H), 4.09 (s, 2H) |
| 49 | 395/396 | (d) δ 8.46 (d, J = 6.0 Hz, 2H), 8.39 (s, 1H), 8.26 (d, J = 1.6 Hz, 1H), |
| | | 8.17 (d, J = 8.4 Hz, 1H), 7.97 (d, J = 8.5 Hz, 2H), 7.88 (d, J = 8.5 Hz, |
| | | 2H), 7.77 (dd, J = 8.4, 1.7 Hz, 1H), 7.28 (d, J = 5.9 Hz, 2H), 4.09 |
| | | (d, J = 6.2 Hz, 2H), 2.84 (s, 3H) |
| 50 | 391/392 | (c) δ 11.32 (s, 1H), 8.56-8.36 (m, 3H), 8.29 (d, J = 8.4 Hz, 1H), 8.06- |
| | | 7.79 (m, 6H), 7.35-7.19 (m, 3H), 6.64 (d, J = 7.1 Hz, 1H), 4.10 (d, J = |
| | | 5.9 Hz, 2H) |
| 51 | 364/365 | (c) δ 8.46 (d, J = 5.7 Hz, 2H), 8.37 (s, 1H), 8.08 (d, J = 2.1 Hz, 1H), |
| | | 8.01 (d, J = 1.6 Hz, 1H), 7.91-7.84 (m, 4H), 7.74 (d, J = 8.6 Hz, 1H), |
| | | 7.67 (dd, J = 8.6, 1.8 Hz, 1H), 7.28 (d, J = 5.6 Hz, 2H), 7.07-7.02 (m, |
| | | 1H), 4.09 (d, J = 6.0 Hz, 2H) |
| 52 | 375/376 | (d) δ 8.98 (dd, J = 4.2, 1.6 Hz, 1H), 8.55-8.34 (m, 5H), 8.18-7.88 (m, |
| | | 6H), 7.59 (dd, J = 8.3, 4.2 Hz, 1H), 7.29 (d, J = 5.7 Hz, 2H), 4.11 |
| | | (d, J = 6.3 Hz, 2H) |
| 53 | 368/369 | (d) δ 8.52-8.26 (m, 3H), 7.86-7.71 (m, 4H), 7.39-6.96 (m, 5H), |
| | | 6.10 (s, 2H), 4.06 (d, J = 6.3 Hz, 2H) |
| 54 | 392/393 | (d) δ 12.42 (s, 1H), 8.54-8.32 (m, 4H), 8.23-8.14 (m, 2H), 8.03- |
| | | 7.77 (m, 5H), 7.28 (d, J = 5.9 Hz, 2H), 4.10 (s, 2H) |
| 55 | 382/383 | (d) δ 8.66-8.22 (m, 3H), 7.80 (s, 4H), 7.38-6.89 (m, 5H), 4.30 (s, |
| | | 4H), 4.07 (d, J = 6.0 Hz, 2H) |
| 56 | 393/394 | (d) δ 8.52-8.32 (m, 3H), 8.18-8.01 (m, 2H), 7.94-7.76 (m, 5H), |
| | | 7.47 (d, J = 8.0 Hz, 1H), 7.28 (d, J = 5.9 Hz, 2H), 4.09 (d, J = 6.0 Hz, |
| | | 2H), 3.44-3.40 (m, 2H), 2.97 (t, J = 6.5 Hz, 2H) |
| 57 | 379/380 | (d) δ 8.69 (s, 1H), 8.46 (dd, J = 4.5, 1.5 Hz, 2H), 8.02-7.53 (m, 8H), |
| | | 7.28 (d, J = 5.9 Hz, 2H), 4.45 (s, 2H), 4.09 (s, 2H) |
| 58 | 363/364 | (b) δ 8.42 (dd, J = 4.6, 1.5 Hz, 2H), 7.94-7.62 (m, 6H), 7.36 (ddd, J = |
| | | 9.4, 5.9, 2.6 Hz, 4H), 6.51 (d, J = 3.1 Hz, 1H), 4.21 (s, 2H) |
| 59 | 378/379 | (d) δ 12.83 (s, 1H), 8.53-8.35 (m, 3H), 7.88 (dt, J = 18.2, 8.5 Hz, 5H), |
| | | 7.73 (s, 1H), 7.45-7.24 (m, 3H), 4.10 (d, J = 6.3 Hz, 2H), 2.53 (s, 3H) |
| 60 | 378/379 | (d) δ 11.26 (s, 1H), 8.68-8.18 (m, 3H), 8.02-7.72 (m, 5H), 7.57- |
| | | 7.19 (m, 5H), 6.53 (s, 1H), 4.08 (d, J = 6.2 Hz, 2H) |
| 61 | 381/382 | (d) δ 8.45 (d, J = 5.3 Hz, 3H), 7.84 (s, 4H), 7.36 (dt, J = 41.2, 7.4 Hz, |
| | | 5H), 4.08 (s, 2H) |
| 62 | 381/382 | (d) δ 11.82 (s, 1H), 8.54-8.29 (m, 3H), 7.89-7.70 (m, 5H), 7.54 (dd, |
| | | J = 8.2, 1.5 Hz, 1H), 7.25 (dd, J = 23.7, 6.8 Hz, 3H), 4.07 (d, J = 5.8 Hz, |
| | | 2H) |
| 63 | 369/370 | (b) δ 7.95 (s, 1H), 7.67-7.65 (m, 2H), 7.30-7.27 (m, 4H), 6.99-6.97 (m, |
| | | 1H), 4.00 (s, 3H), 2.54-2.51 (m, 1H), 1.84-1.73 (m, 5H), 1.43-1.26 (m, |
| | | 5H) |

(a) 500 MHz, CD₃OD.
(b) 400 MHz, CD₃OD.
(c) 500 MHz, DMSO-d₆.
(d) 400 MHz, DMSO-d₆.

The activity of the compounds in Examples 1-63 as PU.1 inhibitors is illustrated in the following assays. The compounds described herein can be tested for efficacy in the treatment or prevention of symptoms or indications of a PU.1-mediated diseases using techniques well known to those in the art.

Biological Activity Assays

### THP-1 CD₁₄ Screening Assay Protocol:

5×10⁴ THP-1 cells were plated per well in a tissue culture-treated 96-well plate. At the time of plating, cells were treated with a final concentration of long/ml PMA and 0.04% DMSO vehicle, concominant with compounds in a 5-point 10-fold dilution per compound. Cells were treated with PMA and DMSO as a control for differentiation without compound treatment, and with only DMSO as a control for no differentiation and no compound treatment. 500 nM IACS-71432 was added as a control compound across all assay plates.

After incubation at 37° C./5% CO₂ for 72 hr, media was collected from the cells and the screening assay was performed using the commercially-available human CD14 SimpleStep ELISA kit from Abcam, according to the following protocol:
1) ELISA kits were taken out to equilibrate to rt.
2) Following the protocol listed in the kit, standards were prepared as 1:1 dilutions from 2 ng/ml.
3) Following the protocol listed in the kit, antibody cocktail was prepared (the volume is contingent on the number of wells required).
4) 50 μl of standards were added to the appropriate wells, thoroughly mizing the standards before adding to each well.
5) 25 μl of Sample Diluent (from kit) was added to all sample wells.
6) Cell plates were centrifuged for 5 min at 300 rcf, with slow acceleration and slow deceleration.
7) 25 μl of media from cell plates was transferred to assay plates containing standards.
8) 50 μl of antibody cocktail was added to all of the wells, standards and samples.
9) Assay plates were sealed tightly and incubated on the plate shaker at 400 rpm for 1 hr at rt.
10) Wash buffer was prepared following the protocol listed in the kit.
11) After the hour incubation, assay plates were washed 5 times using 200 μl wash buffer per well per wash.
12) 100 μl of TMB substrate was added to each well in the dark and incubated on the shaker for 3-4.min, at which point color development was monitored to ensure a robust signal for detection but to avoid oversaturation of signal, for no longer than 10 min.
13) The absorbance at 607 nm was read on a Perkin Elmer Envision plate reader.
14) Data was processed using Microsoft Excel and GraphPad Prism.

TABLE 3
Biological activity of inhibitors in the THP-1 CD14 Assay:
A < 5 nM; B: 5-100 nM; C: 100-500 nM; D: >500 nM.

| Ex. No. | THP-1 CD14 IC₅₀ |
|---|---|
| 1 | B |
| 2 | A |
| 3 | C |
| 4 | C |
| 5 | C |
| 6 | <A |
| 7 | A |
| 8 | A |
| 9 | A |
| 10 | B |
| 11 | A |
| 12 | B |
| 13 | C |
| 14 | A |
| 15 | A |
| 16 | A |
| 17 | A |
| 18 | D |
| 19 | B |
| 20 | A |
| 21 | D |
| 22 | B |
| 23 | B |
| 24 | C |
| 25 | B |
| 26 | B |
| 27 | B |
| 28 | B |
| 29 | B |
| 30 | B |
| 31 | A |
| 32 | B |
| 33 | B |
| 34 | B |
| 35 | <A |
| 36 | A |
| 37 | <A |
| 38 | B |
| 39 | A |
| 40 | A |
| 41 | <A |
| 42 | A |
| 43 | A |
| 44 | A |
| 45 | A |
| 46 | A |
| 47 | A |
| 48 | A |
| 49 (a) | A |
| 49 (b) | C |
| 50 | C |
| 51 | <A |
| 52 | A |
| 53 | A |
| 54 | C |
| 55 | A |
| 56 | B |
| 57 | B |
| 58 | A |
| 59 | A |
| 60 | A |
| 61 | B |
| 62 | C |
| 63 | A |

All references, patents or applications, U.S. or foreign, cited in the application are hereby incorporated by reference as if written herein in their entireties. Where any inconsistencies arise, material literally disclosed herein controls.

From the foregoing description, one skilled in the art can easily ascertain the essential characteristics of this disclosure, and without departing from the spirit and scope thereof, can make various changes and modifications of the disclosure to adapt it to various usages and conditions.

What is claimed is:

1. A compound of structural Formula (I): 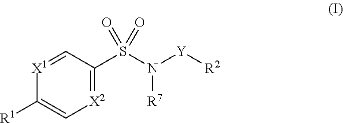
or a pharmaceutically acceptable salt thereof, wherein
Y is a direct bond or CR³R³;
R¹ is chosen from C₁₋₈alkyl, C₁₋₈alkoxy, C₃₋₈cycloalkyl, C₃₋₈cycloalkenyl, C₃₋₈cycloalkoxy, C₆₋₁₀aryl, 5-9 membered heterocycloalkyl, 5-9 membered heterocycloalkyloxy, 5-9 membered heterocycloalkyl(C₁₋₄)alkoxy, and 5-9 membered heteroaryl, each of which is optionally substituted with one or two R⁴ groups;
R² is heteroaryl, optionally substituted with one or two R⁵ groups;
X¹ and X² are chosen from CR₆ and N;
at least one of X¹ and X² is not N;
each occurrence of R³ is independently chosen from H and methyl;
each occurrence of R⁴ is independently chosen from C₁₋₄alkyl, C₁₋₈alkoxy, C₁₋₄alkylcarbonyl, optionally substituted amino, C₃₋₈cycloalkyl, halo, halo C₁₋₄alkyl, halo C₁₋₄alkoxy, hydroxy, and oxo,
wherein C₁₋₄alkyl and C₁₋₈alkoxy can be further optionally substituted with one, two or three groups chosen from C₁₋₄alkoxy, optionally substituted amino, hydroxy, and halo;
each occurrence of R⁵ is independently chosen from C₁₋₄ alkyl, C₁₋₈ alkoxy, optionally substituted amino, acylamino, halo, and hydroxy;
each occurrence of R⁶ is independently chosen from hydrogen, halogen, C₁₋₈ alkyl and C₁₋₈ alkoxy; and
R⁷ is chosen from hydrogen and C₁₋₄ alkyl;
provided that when R¹ is cyclopentyl or cyclohexyl, each of which is optionally substituted with one or two R⁴ groups, and R² is pyridin-4-yl optionally substituted with one or two R⁵ groups, then Y is not CH₂; and
further provided that when R¹ is methoxy or ethoxy and R² is pyridin-4-yl, then Y is not CH₂.

2. A compound of Formula I 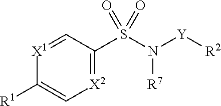
or a pharmaceutically acceptable salt thereof, wherein
Y is a direct bond or CR³R³;
R¹ is chosen from C₁₋₈alkyl, C₃₋₈alkoxy, C₃₋₈cycloalkenyl, C₃₋₈cycloalkoxy, C₆₋₁₀aryl, 5-9 membered heterocycloalkyl, 5-9 membered heterocycloalkyloxy, 5-9 membered heterocycloalkyl(C₁₋₄)alkoxy, and 5-9 membered heteroaryl, each of which is optionally substituted with one or two R⁴ groups or R¹ is C₃₋₈cycloalkyl substituted with one or two R⁴ groups;
R² is heteroaryl, optionally substituted with one or two R⁵ groups;
X¹ and X² are chosen from CR⁶, and N, provided that at least one of X¹ and X² is not N;
each occurrence of R³ is independently chosen from H and methyl;
each occurrence of R⁴ is independently chosen from C₁₋₄alkyl, C₁₋₈alkoxy, C₁₋₄alkylcarbonyl, optionally substituted amino, C₃₋₈cycloalkyl, halo,
halo C₁₋₄ alkyl, halo C₁₋₄alkoxy, hydroxy, and oxo,
wherein C₁₋₄alkyl and C₁₋₈alkoxy can be further optionally substituted with one, two or three groups chosen from C₁₋₄alkoxy, optionally substituted amino, hydroxy, and halo;
each occurrence of R⁵ is independently chosen from C₁₋₄ alkyl, C₁₋₈ alkoxy, optionally substituted amino, halo, and hydroxy;
each occurrence of R⁶ is independently chosen from hydrogen, C₁₋₈ alkyl and C₁₋₈ alkoxy; and
R⁷ is chosen from hydrogen and C₁₋₄ alkyl.

3. The compound of claim 1 or 2, or a pharmaceutically acceptable salt thereof, wherein R¹ is C₁₋₈alkyl, optionally substituted with one or two R⁴ groups.

4. The compound of claim 3, or a pharmaceutically acceptable salt thereof, wherein R¹ is C₁₋₄alkyl.

5. The compound of claim 4, or a pharmaceutically acceptable salt thereof, wherein R¹ is 2-propyl.

6. The compound of claim 1, or a pharmaceutically acceptable salt thereof, wherein R¹ is C₁₋₈alkoxy, optionally substituted with one or two R⁴ groups.

7. The compound of claim 6, or a pharmaceutically acceptable salt thereof, wherein R¹ is chosen from ethoxy, 2-(trifluoromethoxy)ethoxy, n-propoxy, 3-hydroxypropoxy, 3-methoxypropoxy, 3-(trifluoromethoxy)propoxy, 2-methoxyethoxy, 2-n-butoxy, n-pentoxy, and 3-hydroxy-3-methylbutoxy.

8. The compound of claim 1 or 2, or a pharmaceutically acceptable salt thereof, wherein R¹ is C₃₋₈alkoxy, optionally substituted with one or two R⁴ groups.

9. The compound of claim 8, or a pharmaceutically acceptable salt thereof, wherein R¹ is chosen from n-propoxy, 3-hydroxypropoxy, 3-methoxypropoxy, 3-(trifluoromethoxy)propoxy, 2-methoxyethoxy, 2-n-butoxy, n-pentoxy, and 3-hydroxy-3-methylbutoxy.

10. The compound of claim 1, or a pharmaceutically acceptable salt thereof, wherein R¹ is cycloalkyl, optionally substituted with one or two R⁴ groups.

11. The compound of claim 10, wherein R¹ is chosen from cyclobutyl, cyclopentyl, and cyclohexyl, optionally substituted with one or two R⁴ groups.

12. The compound of claim 2, or a pharmaceutically acceptable salt thereof, wherein R¹ is C₃₋₅cycloalkyl substituted with one or two R⁴ groups.

13. The compound of claim 10, wherein R¹ is chosen from cyclobutyl, cyclopentyl, cyclohexyl,

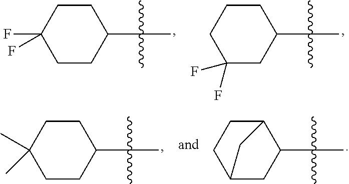

14. The compound of claim 1 or 2, or a pharmaceutically acceptable salt thereof, wherein R¹ is C₃₋₅cycloalkenyl, optionally substituted with one or two R⁴ groups.

15. The compound of claim 14, wherein R¹ is chosen from

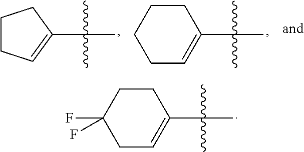

16. The compound of claim 1 or 2, or a pharmaceutically acceptable salt thereof, wherein R¹ is C₁₋₈cycloalkoxy, optionally substituted with one or two R⁴ groups.

17. The compound of claim 16, or a pharmaceutically acceptable salt thereof, wherein R¹ is chosen from

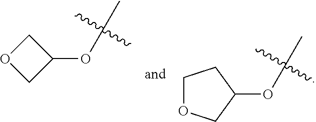

18. The compound of claim 1 or 2, or a pharmaceutically acceptable salt thereof, wherein R¹ is aryl, optionally substituted with one or two R⁴ groups.

19. The compound of claim 18, or a pharmaceutically acceptable salt thereof, wherein R¹ is phenyl, optionally substituted with one or two R⁴ groups.

20. The compound of claim 1 or 2, or a pharmaceutically acceptable salt thereof, wherein R¹ is phenyl, 3-ethyl-4-ethoxyphenyl, 3-methyl-4-propoxyphenyl, 3-methyl-4-(2-methoxyethoxy)phenyl, 3-methyl-4-(2-(dimethylamino)ethoxy)phenyl, 3-cyclopropyl-4-ethoxyphenyl, 3,3-dimethyl-2,3-dihydro-1H-inden-5-yl, 3-oxo-2,3-dihydro-1H-inden-5-yl, and bicyclo[4.2.0]octa-1,3,5-trien-3-yl.

21. The compound of claim 1 or 2, or a pharmaceutically acceptable salt thereof, wherein R¹ is 5-9 membered heterocycloalkyl group chosen from: 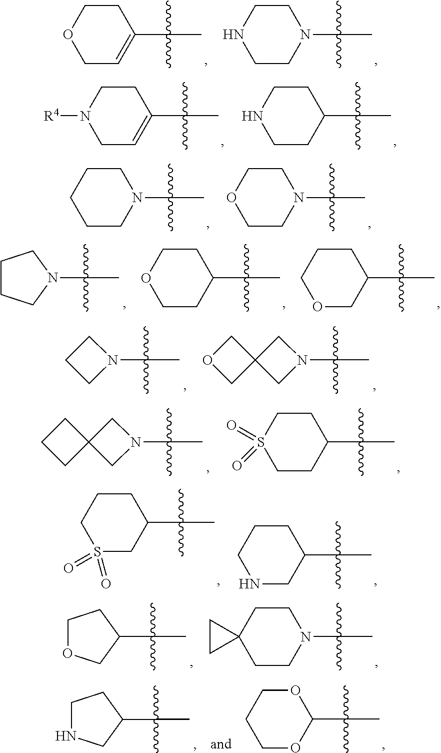
each of which is optionally substituted with one or two R⁴ groups.

22. The compound of claim 1 or 2, or a pharmaceutically acceptable salt thereof, wherein R¹ is 5-9 membered heterocycloalkyl group chosen from:

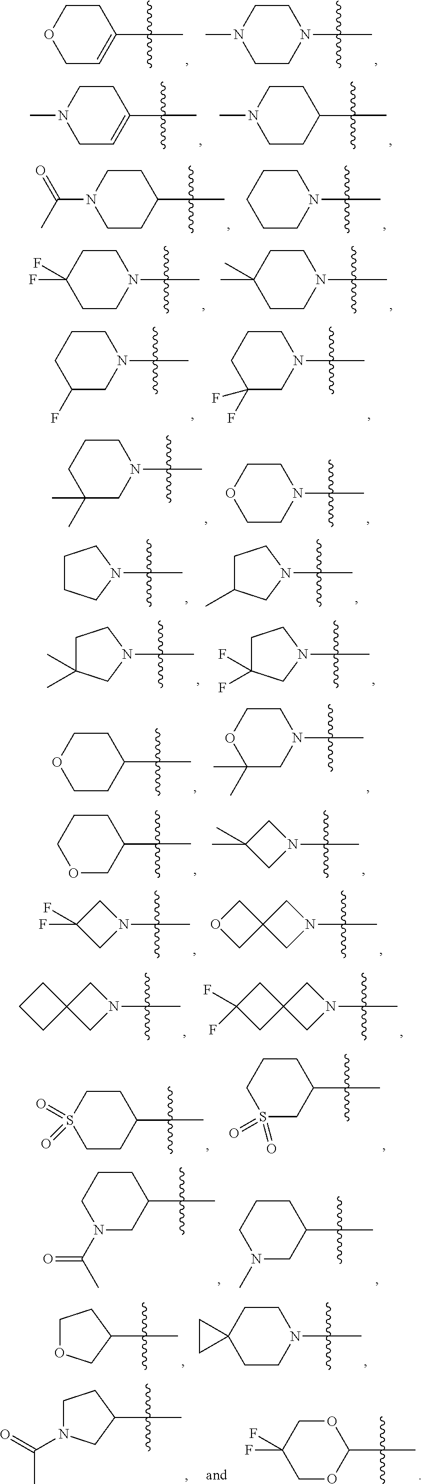

23. The compound of claim 1 or 2, or a pharmaceutically acceptable salt thereof, wherein R¹ is −9 membered heterocycloalkyl(C₁₋₄)alkoxy.

24. The compound of claim 1 or 2, or a pharmaceutically acceptable salt thereof, wherein R¹ is chosen from

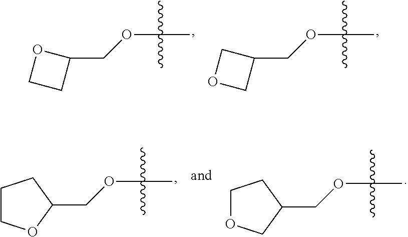

25. The compound of claim 1 or 2, or a pharmaceutically acceptable salt thereof, wherein R¹ is heteroaryl, optionally substituted with one or two R⁴ groups.

26. The compound of claim 1 or 2, or a pharmaceutically acceptable salt thereof, wherein R¹ is chosen from:

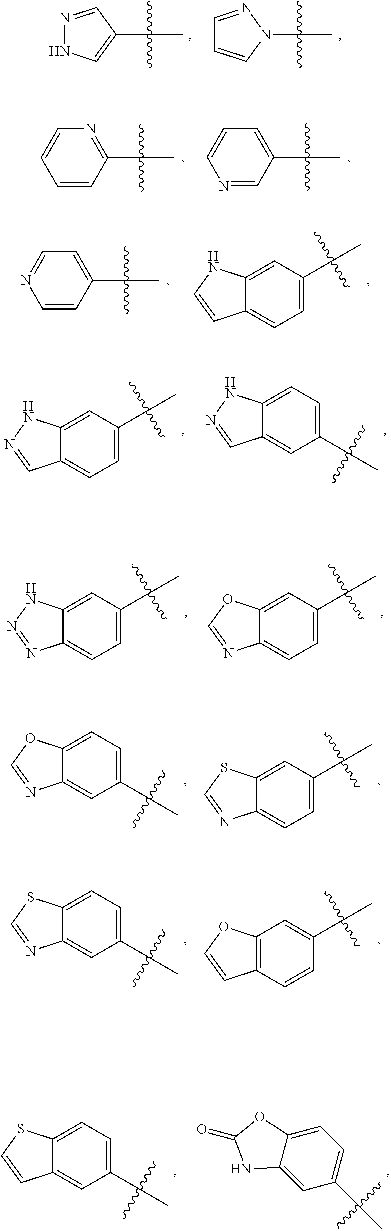

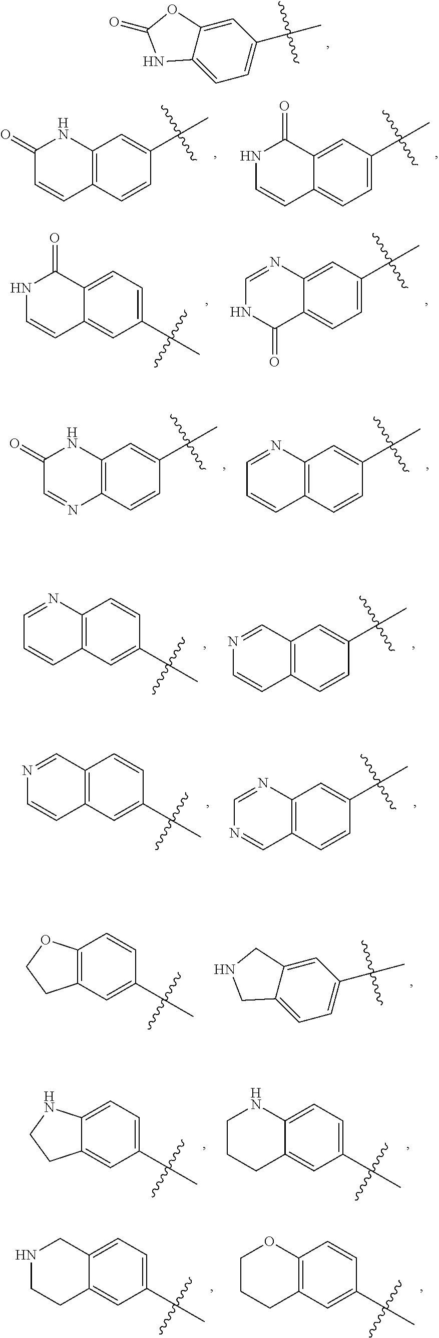

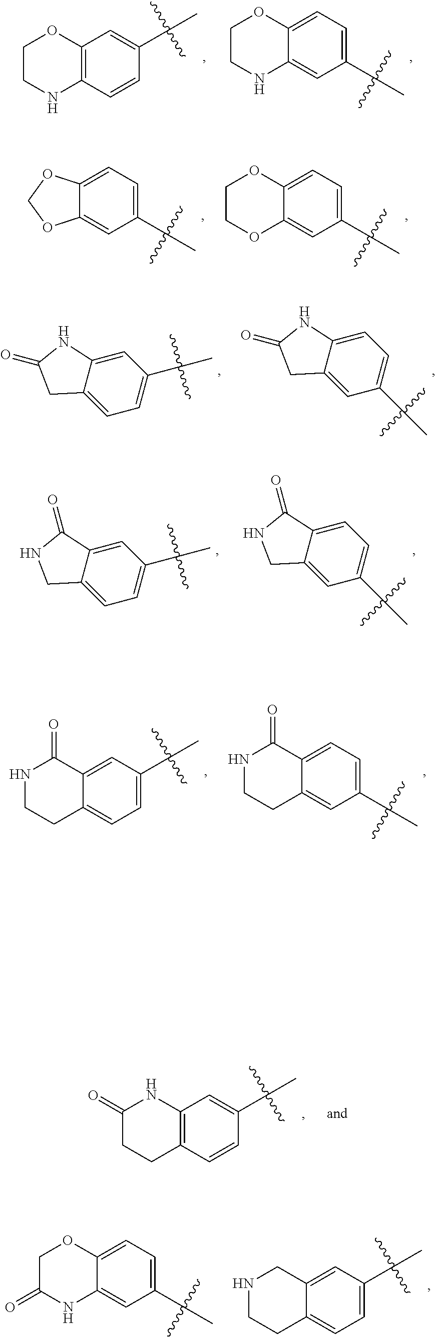

27. The compound of claim 1 or 2, or a pharmaceutically acceptable salt thereof, wherein R¹ is chosen from:

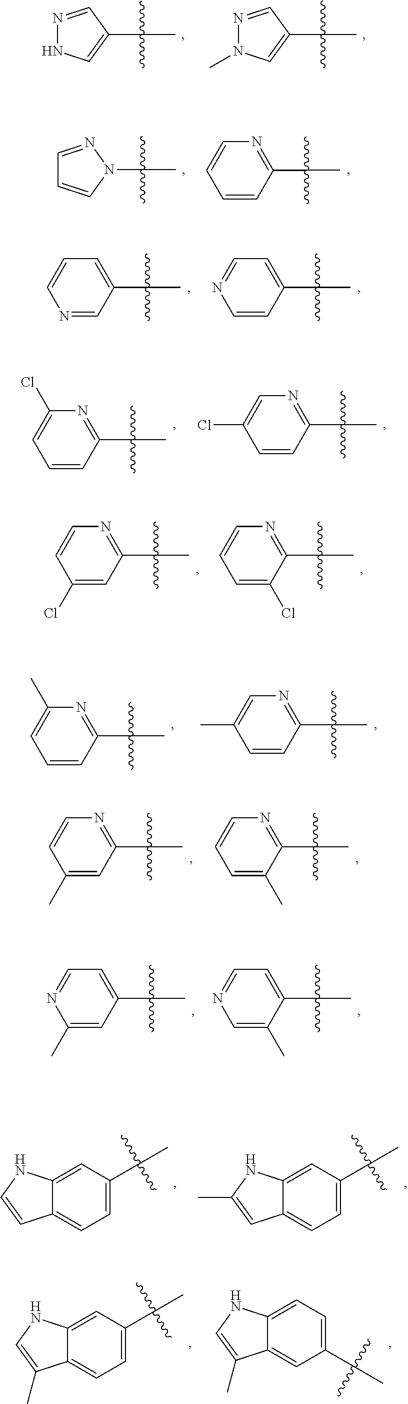

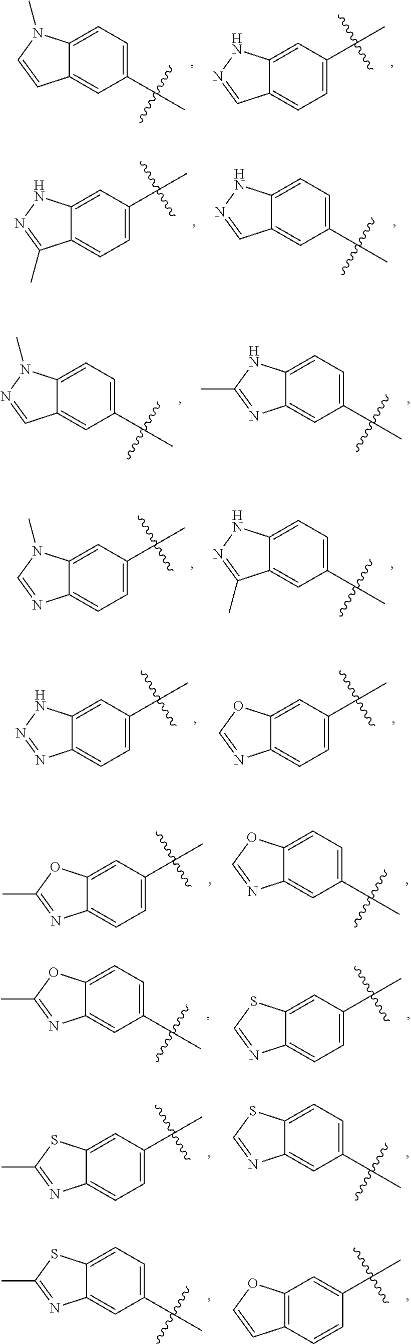

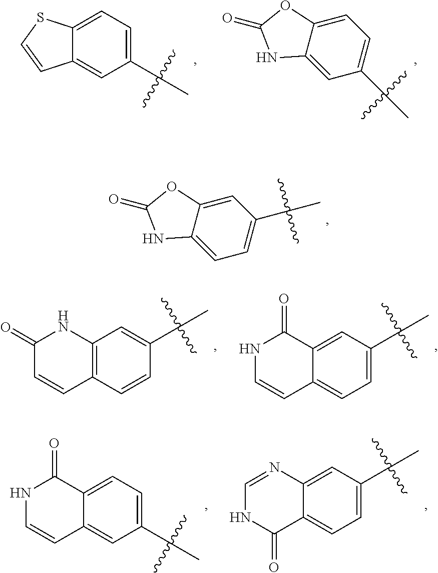

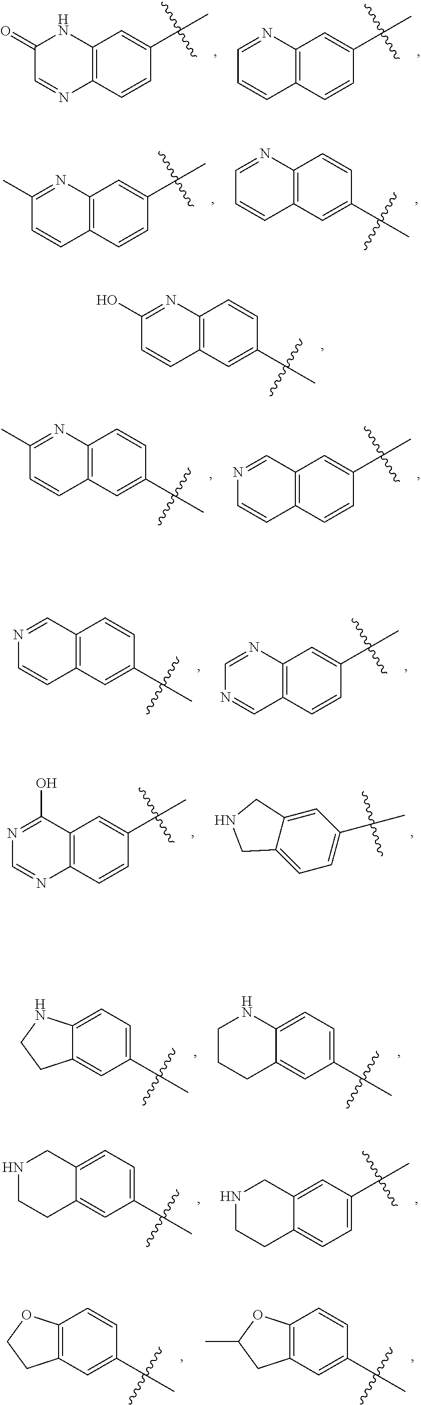

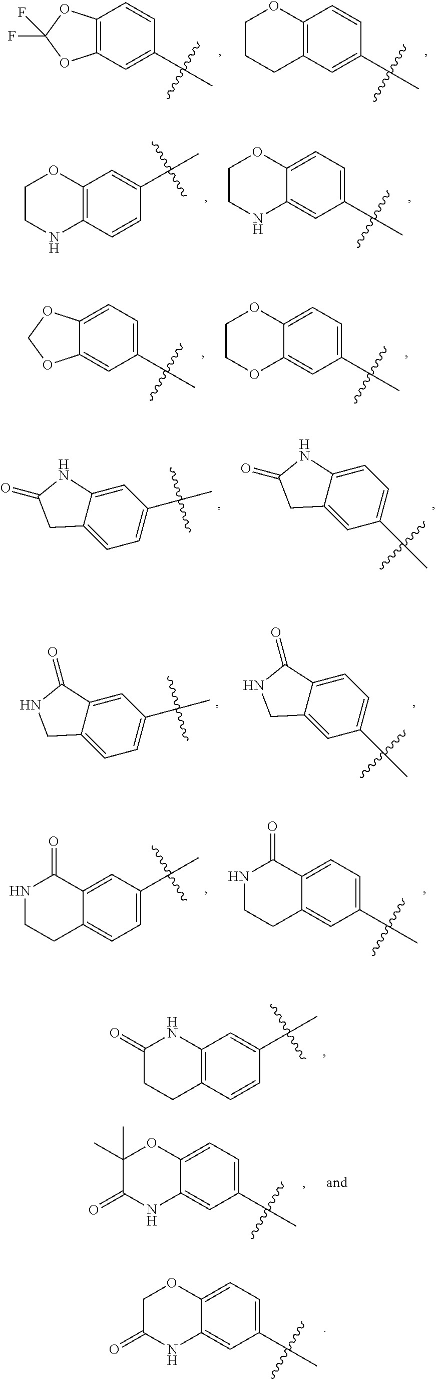

28. The compound of any one of the preceding claims, or a pharmaceutically acceptable salt thereof, wherein R² is chosen from pyridin-4-yl, isoquinolin-5-yl, 1H-indazol-7-yl, 1H-indazol-4-yl, 1H-pyrrolo[2,3-b]pyridin-4-yl, 1-methyl-1H-pyrazol-5-yl, pyridin-4-yl, 2-fluoropyridin-4-yl, 3-fluoropyridin-4-yl, 2-aminopyridin-4-yl, 3-aminopyridin-4-yl, 2-methoxypyridin-4-yl, 3-methoxypyridin-4-yl, 2-pentoxypyridin-4-yl, 3-pentoxypyridin-4-yl, 2-chloropyridin-4-yl, 3-chloropyridin-4-yl, 2-methylpyridin-4-yl, 3-methylpyridin-4-yl, isoquinolin-5-yl, 3-chloro-1-methyl-1H-indazol-7-yl, 3-chloro-1H-indazol-7-yl, 1-methyl-1H-indazol-4-yl, 1-methyl-1H-indol-4-yl, 3-methyl-1H-indol-7-yl, 1-methyl-1H-pyrrolo[2,3-b]pyridin-4-yl, and 1H-pyrrolo[2,3-b]pyridin-4-yl, each of which is optionally substituted with one or two R⁵ groups.

29. The compound of claim 1 or 2, or a pharmaceutically acceptable salt thereof, wherein R² is chosen from 1-methyl-1H-pyrazol-5-yl, pyridin-4-yl, 2-fluoropyridin-4-yl, 3-fluoropyridin-4-yl, 2-aminopyridin-4-yl, 3-aminopyridin-4-yl, 2-methoxypyridin-4-yl, 3-methoxypyridin-4-yl, 2-pentoxypyridin-4-yl, 3-pentoxypyridin-4-yl, 2-chloropyridin-4-yl, 3-chloropyridin-4-yl, 2-methylpyridin-4-yl, 3-methylpyridin-4-yl, isoquinolin-5-yl, 3-chloro-1-methyl-1H-indazol-7-yl, 3-chloro-1H-indazol-7-yl, 1-methyl-1H-indazol-4-yl, 1-methyl-1H-indol-4-yl, 3-methyl-1H-indol-7-yl, 1-methyl-1H-pyrrolo[2,3-b]pyridin-4-yl, and 1H-pyrrolo[2,3-b]pyridin-4-yl.

30. The compound of claim 1 or 2, or a pharmaceutically acceptable salt thereof, wherein R² is pyridin-4-yl, optionally substituted with one R⁵ groups.

31. The compound of claim 31, or a pharmaceutically acceptable salt thereof, wherein R² is pyridin-4-yl.

32. The compound of any one of claims 1-31, or a pharmaceutically acceptable salt thereof, wherein Y is a direct bond.

33. The compound of any one of claims 1-32, or a pharmaceutically acceptable salt thereof, wherein Y is CR³R³.

34. The compound of claim 33, or a pharmaceutically acceptable salt thereof, wherein at least one occurrence of R³ is H.

35. The compound of claim 33, or a pharmaceutically acceptable salt thereof, wherein both occurrences of R³ are H.

36. The compound of claim 33, or a pharmaceutically acceptable salt thereof, wherein one occurrence of R³ is H and the other is methyl.

37. The compound of claim 33, or a pharmaceutically acceptable salt thereof, wherein both occurrences of R³ are methyl.

38. The compound of any one of claims 1-37, or a pharmaceutically acceptable salt thereof, wherein each occurrence R⁴ is independently chosen from methyl, methoxy, hydroxy, oxo, chloro, and fluoro.

39. The compound of any one of claims 1-38, or a pharmaceutically acceptable salt thereof, wherein each occurrence R⁵ is independently chosen from chloro, fluoro, methyl, and methoxy.

40. The compound of claim 1, or a pharmaceutically acceptable salt thereof, wherein the compound of Formula I is chosen from:

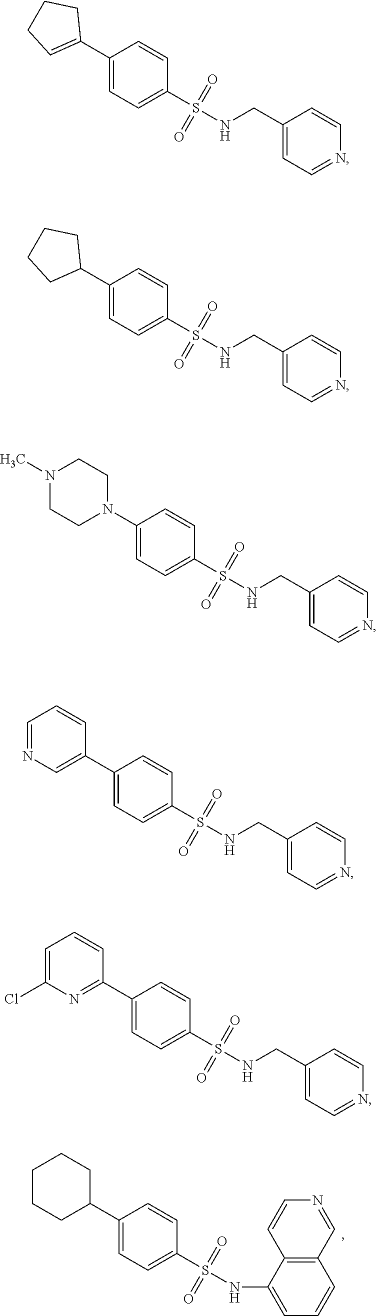 [SMILES: O=S(=O)(NCc1ccncc1)c1ccc(C2=CCCC2)cc1 ; O=S(=O)(NCc1ccncc1)c1ccc(C2CCCC2)cc1 ; CN1CCN(c2ccc(S(=O)(=O)NCc3ccncc3)cc2)CC1 ; O=S(=O)(NCc1ccncc1)c1ccc(-c2cccnc2)cc1 ; O=S(=O)(NCc1ccncc1)c1ccc(-c2cccc(Cl)n2)cc1 ; O=S(=O)(Nc1cccc2cnccc12)c1ccc(C2CCCCC2)cc1]

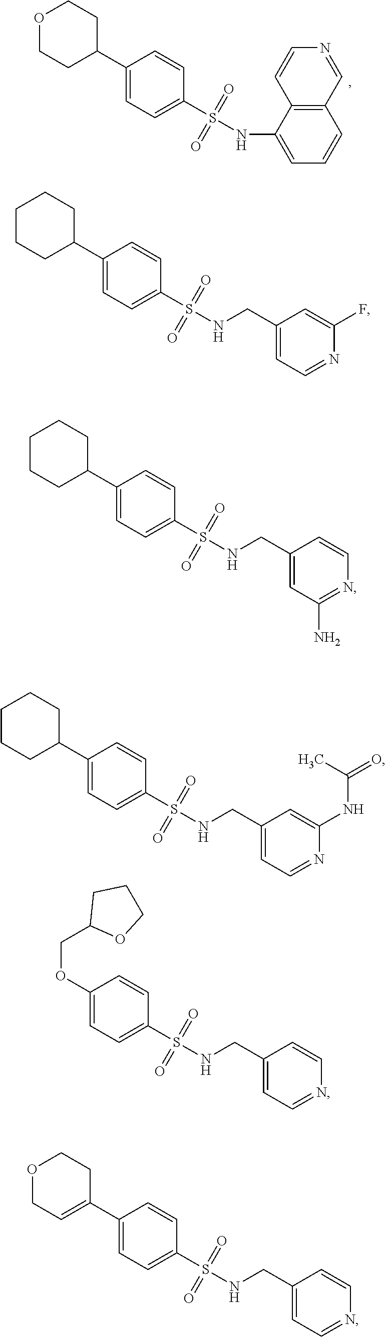 [SMILES: O=S(=O)(Nc1cccc2cnccc12)c1ccc(C2CCOCC2)cc1 ; O=S(=O)(NCc1ccnc(F)c1)c1ccc(C2CCCCC2)cc1 ; Nc1cc(CNS(=O)(=O)c2ccc(C3CCCCC3)cc2)ccn1 ; CC(=O)Nc1cc(CNS(=O)(=O)c2ccc(C3CCCCC3)cc2)ccn1 ; O=S(=O)(NCc1ccncc1)c1ccc(OCC2CCCO2)cc1 ; O=S(=O)(NCc1ccncc1)c1ccc(C2=CCOCC2)cc1]

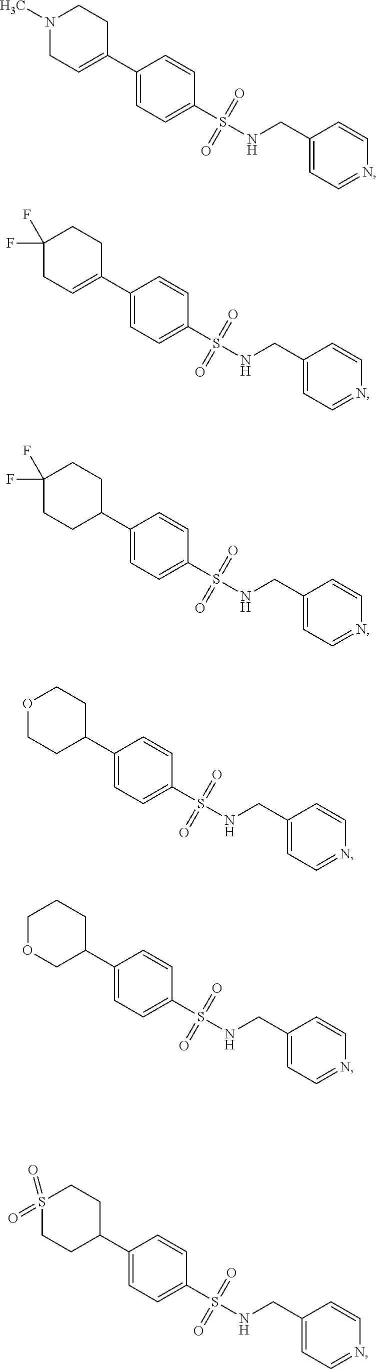 [SMILES: CN1CC=C(c2ccc(S(=O)(=O)NCc3ccncc3)cc2)CC1 ; O=S(=O)(NCc1ccncc1)c1ccc(C2=CCC(F)(F)CC2)cc1 ; O=S(=O)(NCc1ccncc1)c1ccc(C2CCC(F)(F)CC2)cc1 ; O=S(=O)(NCc1ccncc1)c1ccc(C2CCOCC2)cc1 ; O=S(=O)(NCc1ccncc1)c1ccc(C2CCCOC2)cc1 ; O=S1(=O)CCC(c2ccc(S(=O)(=O)NCc3ccncc3)cc2)CC1]

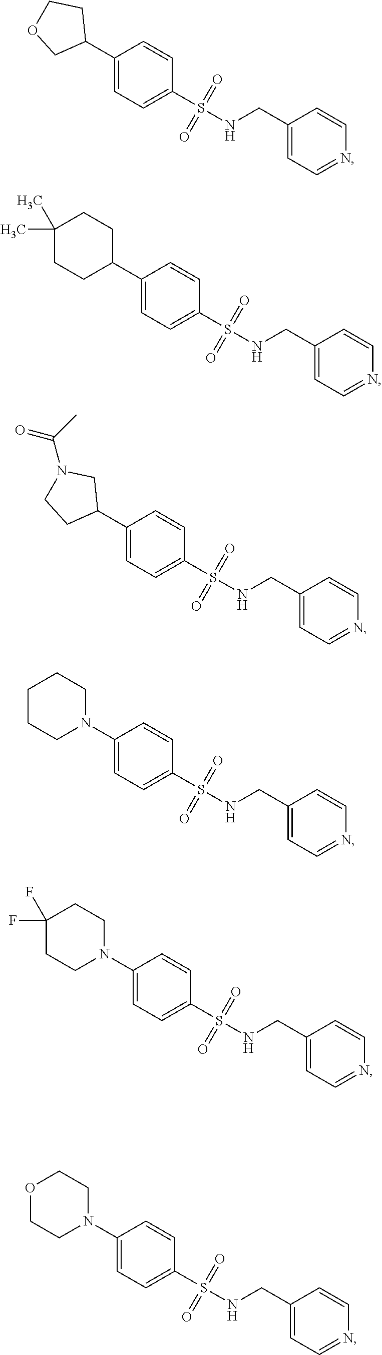 [SMILES: O=S(=O)(NCc1ccncc1)c1ccc(C2CCOC2)cc1 ; CC1(C)CCC(c2ccc(S(=O)(=O)NCc3ccncc3)cc2)CC1 ; CC(=O)N1CCC(c2ccc(S(=O)(=O)NCc3ccncc3)cc2)C1 ; O=S(=O)(NCc1ccncc1)c1ccc(N2CCCCC2)cc1 ; O=S(=O)(NCc1ccncc1)c1ccc(N2CCC(F)(F)CC2)cc1 ; O=S(=O)(NCc1ccncc1)c1ccc(N2CCOCC2)cc1]

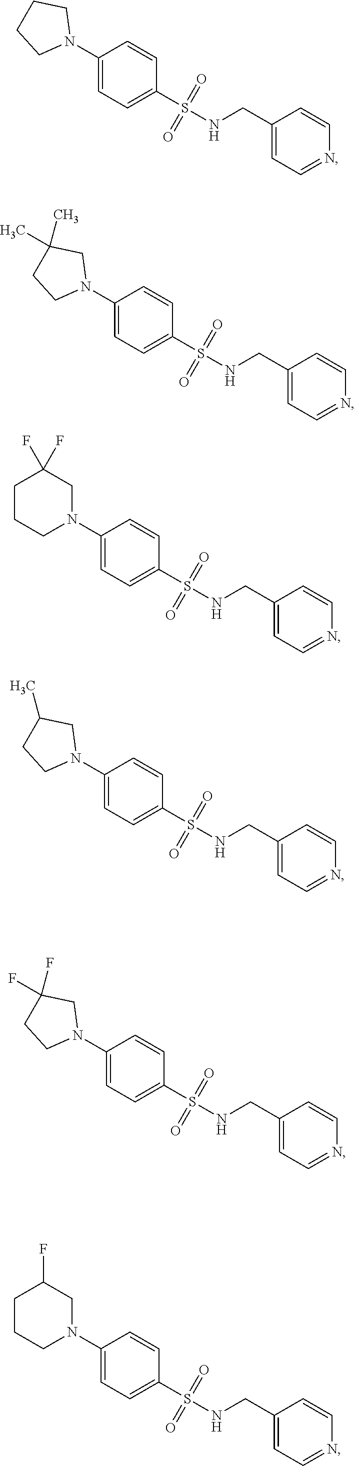 [SMILES: O=S(=O)(NCc1ccncc1)c1ccc(N2CCCC2)cc1 ; CC1(C)CCN(c2ccc(S(=O)(=O)NCc3ccncc3)cc2)C1 ; O=S(=O)(NCc1ccncc1)c1ccc(N2CCCC(F)(F)C2)cc1 ; CC1CCN(c2ccc(S(=O)(=O)NCc3ccncc3)cc2)C1 ; O=S(=O)(NCc1ccncc1)c1ccc(N2CCC(F)(F)C2)cc1 ; O=S(=O)(NCc1ccncc1)c1ccc(N2CCCC(F)C2)cc1]

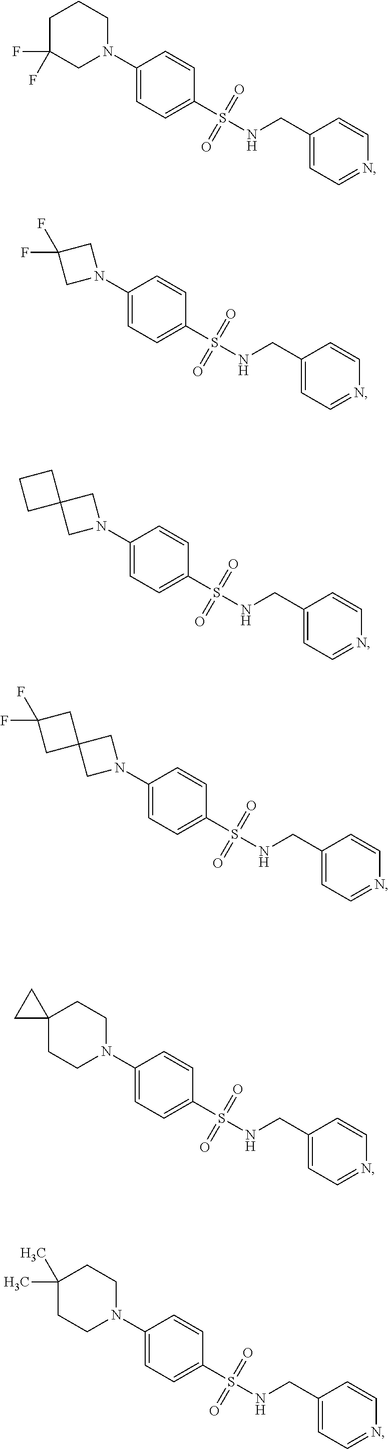 [SMILES: O=S(=O)(NCc1ccncc1)c1ccc(N2CCCC(F)(F)C2)cc1 ; O=S(=O)(NCc1ccncc1)c1ccc(N2CC(F)(F)C2)cc1 ; O=S(=O)(NCc1ccncc1)c1ccc(N2CC3(CCC3)C2)cc1 ; O=S(=O)(NCc1ccncc1)c1ccc(N2CC3(C2)CC(F)(F)C3)cc1 ; O=S(=O)(NCc1ccncc1)c1ccc(N2CCC3(CC2)CC3)cc1 ; CC1(C)CCN(c2ccc(S(=O)(=O)NCc3ccncc3)cc2)CC1]

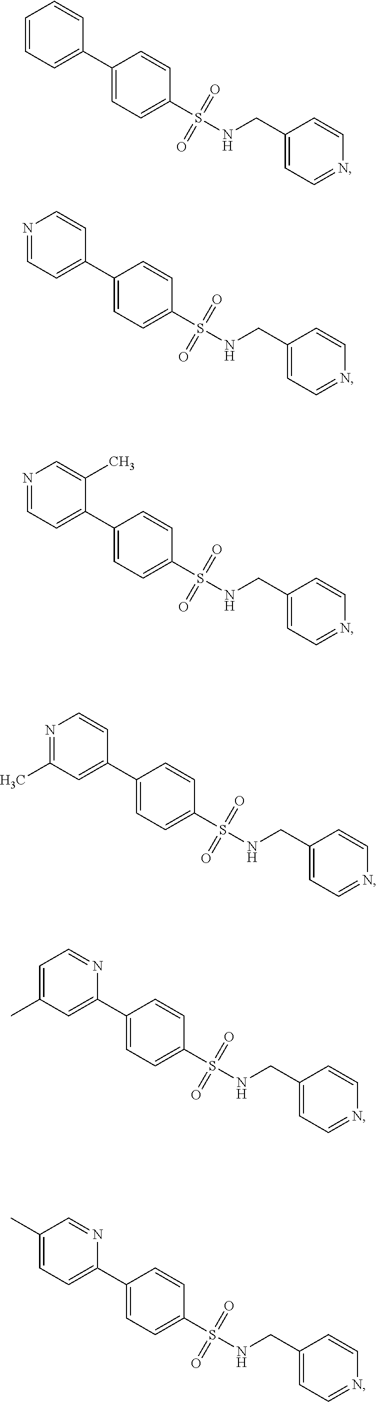 [SMILES: O=S(=O)(NCc1ccncc1)c1ccc(-c2ccccc2)cc1 ; O=S(=O)(NCc1ccncc1)c1ccc(-c2ccncc2)cc1 ; Cc1cnccc1-c1ccc(S(=O)(=O)NCc2ccncc2)cc1 ; Cc1cc(-c2ccc(S(=O)(=O)NCc3ccncc3)cc2)ccn1 ; Cc1ccnc(-c2ccc(S(=O)(=O)NCc3ccncc3)cc2)c1 ; Cc1ccc(-c2ccc(S(=O)(=O)NCc3ccncc3)cc2)nc1]

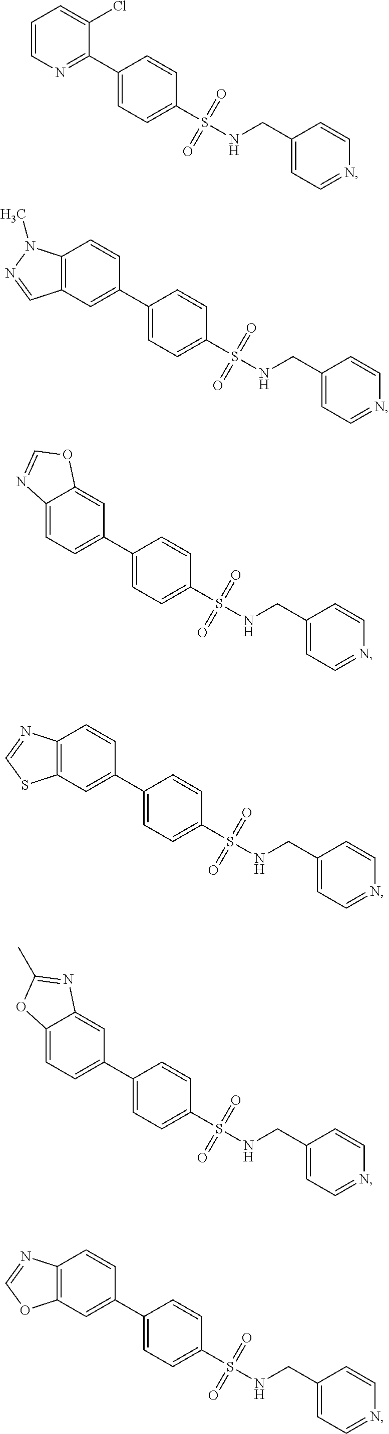 [SMILES: O=S(=O)(NCc1ccncc1)c1ccc(-c2ncccc2Cl)cc1 ; Cn1ncc2cc(-c3ccc(S(=O)(=O)NCc4ccncc4)cc3)ccc21 ; O=S(=O)(NCc1ccncc1)c1ccc(-c2ccc3ncoc3c2)cc1 ; O=S(=O)(NCc1ccncc1)c1ccc(-c2ccc3ncsc3c2)cc1 ; Cc1nc2cc(-c3ccc(S(=O)(=O)NCc4ccncc4)cc3)ccc2o1 ; O=S(=O)(NCc1ccncc1)c1ccc(-c2ccc3ncoc3c2)cc1]

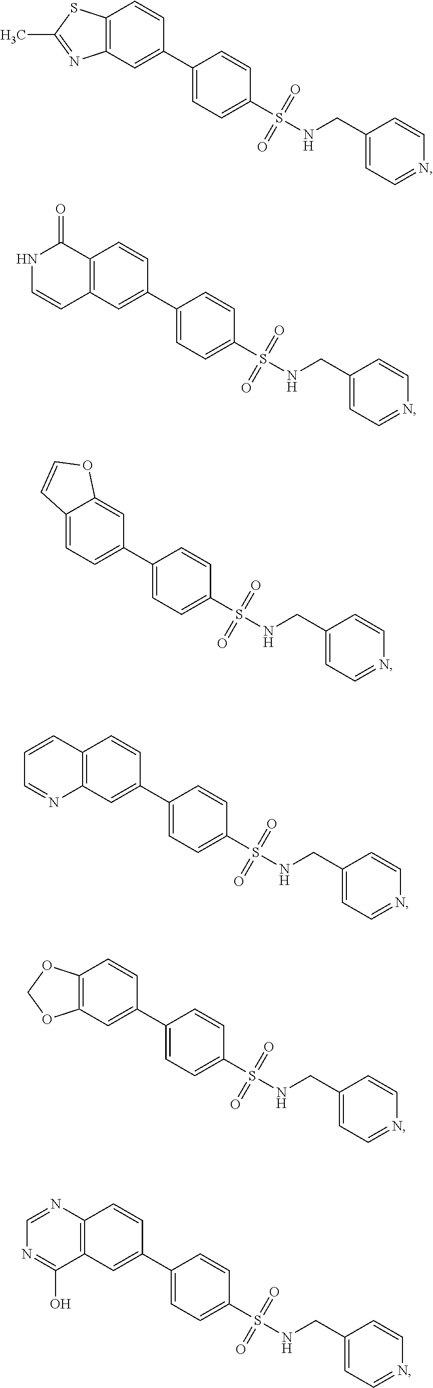 [SMILES: Cc1nc2cc(-c3ccc(S(=O)(=O)NCc4ccncc4)cc3)ccc2s1 ; O=c1[nH]ccc2cc(-c3ccc(S(=O)(=O)NCc4ccncc4)cc3)ccc12 ; O=S(=O)(NCc1ccncc1)c1ccc(-c2ccc3ccoc3c2)cc1 ; O=S(=O)(NCc1ccncc1)c1ccc(-c2ccc3cccnc3c2)cc1 ; O=S(=O)(NCc1ccncc1)c1ccc(-c2ccc3c(c2)OCO3)cc1 ; O=S(=O)(NCc1ccncc1)c1ccc(-c2ccc3ncnc(O)c3c2)cc1]

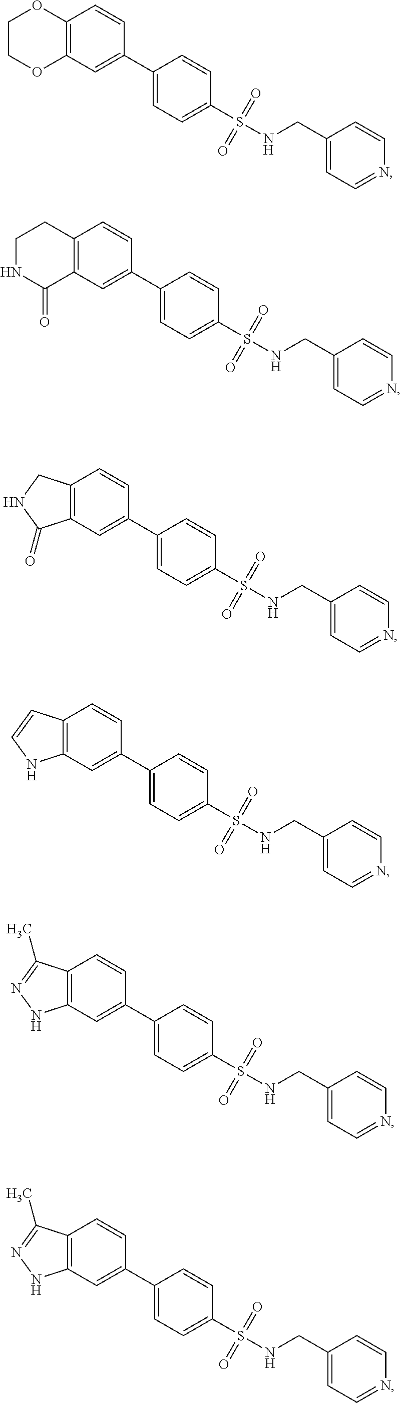 [SMILES: O=S(=O)(NCc1ccncc1)c1ccc(-c2ccc3c(c2)OCCO3)cc1 ; O=C1NCCc2ccc(-c3ccc(S(=O)(=O)NCc4ccncc4)cc3)cc21 ; O=C1NCc2ccc(-c3ccc(S(=O)(=O)NCc4ccncc4)cc3)cc21 ; O=S(=O)(NCc1ccncc1)c1ccc(-c2ccc3cc[nH]c3c2)cc1 ; Cc1n[nH]c2cc(-c3ccc(S(=O)(=O)NCc4ccncc4)cc3)ccc12 ; Cc1n[nH]c2cc(-c3ccc(S(=O)(=O)NCc4ccncc4)cc3)ccc12]

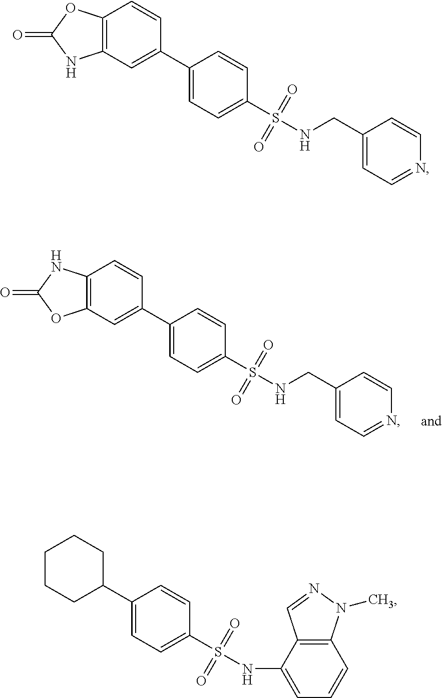 [SMILES: O=c1[nH]c2cc(-c3ccc(S(=O)(=O)NCc4ccncc4)cc3)ccc2o1 ; O=c1[nH]c2ccc(-c3ccc(S(=O)(=O)NCc4ccncc4)cc3)cc2o1 ; Cn1ncc2c(NS(=O)(=O)c3ccc(C4CCCCC4)cc3)cccc21]

41. A pharmaceutical formulation comprising a compound of any one of claims 1-40, or a pharmaceutically acceptable salt thereof, and a pharmaceutically acceptable carrier.

42. A method of treatment of a PU.1-mediated disease comprising the administration of a therapeutically effective amount of a compound as recited in any one of claims 1-40, or a pharmaceutically acceptable salt thereof, to a patient in need thereof.

43. The method as recited in claim 42, or a pharmaceutically acceptable salt thereof, wherein the disease is chosen from multiple sclerosis, Parkinson's Disease, Huntington's Disease, amyotrophic lateral sclerosis, neuroinflammation, frontotemporal dementia, dementia with Lewy bodies, neuropathic pain, inflammatory pain, neuropathic itch, inflammatory itch, neuropathic dysesthesia, inflammatory dysesthesia, dementia, glioma, brain tumors, Batten disease, Down's Syndrome, Nasu-Hakola, prion disease, Cockayne syndrome, Ataxia-telangiectasia, xeroderma pigmentosum, schizophrenia, bipolar disorder, epilepsy, motor neuron disease, sciatica, Friedreich's ataxia, Gerstmann-Straussler-Scheinker Disease, Kuru, Alper's Disease, apnea, corticobasal degeneration, Leigh's Disease, Monomelic amyotrophy, multiple system atrophy, multiple system atrophy with orthostatic hypotension, narcolepsy, neurodegeneration with brain iron accumulation, opsoclonus myoclonus, progressive multifocal leukoencephalopathy, strationigral degeneration, transmissible spongiform encephalopathis, ataxia, Sjogren's disease, Sandhoff disease, Myasthenia gravis, Tay-Sachs disease, neuronal ceroid lipofuscinosis, senesence, progeria, sepsis, Lyme disease, leukemia, lupus, fibrosis, cancer, hematologic cancer, bone cancer, glioblastomas, inflammatory diseases, inflammatory disorders, autoimmune disorders, endotoxemia and neurodegenerative diseases, including without limitation, such conditions ase acute myeloid leukemia, rheumatoid arthritis, contact dermatitis, asthma, inflammatory bowel disease, pediatric atrophy, giant cell arteritis, Alzheimer's disease, and systemic lupus.

44. The method as recited in claim 42, wherein the disease is chosen from Alzheimer's disease, inflammation, or excessive myelin uptake.

45. The method as recited in claim 43, wherein the disease is Alzheimer's disease.

46. The method as recited in any one of claims 41-44, further comprising the administration of another therapeutic agent.
